SEQUENCE LISTING (31 sequences)

SEQ ID NO: 1 - DNA, 20 bp, Artificial Sequence
  Synthetic
ccctgttcactgtgagtagg

SEQ ID NO: 2 - DNA, 22 bp, Artificial Sequence
  Synthetic
gcgatccctgaacatgtccatc

SEQ ID NO: 3 - DNA, 22 bp, Artificial Sequence
  Synthetic
agtatctggtaggtgcttccag

SEQ ID NO: 4 - DNA, 23 bp, Artificial Sequence
  Synthetic
tctgtgaagcgagtccttaggct

SEQ ID NO: 5 - DNA, 23 bp, Artificial Sequence
  Synthetic
cgcactctctctctcccaaggaa

SEQ ID NO: 6 - DNA, 19 bp, Artificial Sequence
  Synthetic
cccgcagaacctgaagatg

SEQ ID NO: 7 - DNA, 19 bp, Artificial Sequence
  Synthetic
gacccggcaaaacaggtag

SEQ ID NO: 8 - DNA, 20 bp, Artificial Sequence
  Synthetic
ctgggagcgcaggttaaata

SEQ ID NO: 9 - DNA, 26 bp, Artificial Sequence
  Synthetic
gccaataattaaaatactgcaggaaa

SEQ ID NO: 10 - DNA, 23 bp, Artificial Sequence
  Synthetic
atatctcacgtactgacggtggg

SEQ ID NO: 11 - DNA, 23 bp, Artificial Sequence
  Synthetic
ctgtttcactatccaggttacgg

SEQ ID NO: 12 - DNA, 20 bp, Artificial Sequence
  Synthetic
aggtaaagtctcctacgcgg

SEQ ID NO: 13 - DNA, 20 bp, Artificial Sequence
  Synthetic
cttccaaacctgaaccgccc

SEQ ID NO: 14 - DNA, 20 bp, Artificial Sequence
  Synthetic
tttctgctctgcagcacact

SEQ ID NO: 15 - DNA, 22 bp, Artificial Sequence
  Synthetic
cagacaacataaactgcgcctt

SEQ ID NO: 16 - DNA, 21 bp, Artificial Sequence
  Synthetic
ttcaggcaggcagtatcactc

SEQ ID NO: 17 - DNA, 20 bp, Artificial Sequence
  Synthetic
tctccaccgcaatgaagacc

SEQ ID NO: 18 - DNA, 20 bp, Artificial Sequence
  Synthetic
ttgtgctccttgtcaacagc

SEQ ID NO: 19 - DNA, 19 bp, Artificial Sequence
  Synthetic
atgagcacagaaagcatga

SEQ ID NO: 20 - DNA, 22 bp, Artificial Sequence
  Synthetic
aatgtgtccgtcgtggatctga

SEQ ID NO: 21 - DNA, 20 bp, Artificial Sequence
  Synthetic
gattgtctccatgtcgttgc

SEQ ID NO: 22 - DNA, 23 bp, Artificial Sequence
  Synthetic
gatacacctctccaccaatgacc

SEQ ID NO: 23 - DNA, 21 bp, Artificial Sequence
  Synthetic
cgtcacacaccagcaggttat

SEQ ID NO: 24 - DNA, 20 bp, Artificial Sequence
  Synthetic
cacacccaccagcatcttct

SEQ ID NO: 25 - DNA, 20 bp, Artificial Sequence
  Synthetic
ctggggagtttcaggttcct

SEQ ID NO: 26 - DNA, 20 bp, Artificial Sequence
  Synthetic
agtagacagaagagcgtggt

SEQ ID NO: 27 - DNA, 22 bp, Artificial Sequence
  Synthetic
agtgtagcccaagatgcccttc

SEQ ID NO: 28 - DNA, 24 bp, Artificial Sequence
  Synthetic
tggacgcagacgatgtctttacca

SEQ ID NO: 29 - DNA, 24 bp, Artificial Sequence
  Synthetic
acatggccatgcctgggtctgaga

SEQ ID NO: 30 - DNA, 24 bp, Artificial Sequence
  Synthetic
accagagacggaaatccatcgctc

SEQ ID NO: 31 - DNA, 24 bp, Artificial Sequence
  Synthetic
tgccacgaccaagtgacagcaatg

(19) United States
(12) Patent Application Publication
(10) Pub. No.: US 2023/0151075 A1
(43) Pub. Date: May 18, 2023

(54) PARATHYROID HORMONE ATTENUATES LOW BACK PAIN AND OSTEOARTHRITIC PAIN

(71) Applicant: The Johns Hopkins University, Baltimore, MD (US)

(72) Inventor: Xu Cao, Baltimore, MD (US)

(21) Appl. No.: 17/781,185

(22) PCT Filed: Dec. 2, 2020
(86) PCT No.: PCT/US2020/062879

§ 371 Date: May 31, 2022

Related U.S. Application Data
(60) Provisional application No. 62/942,945, filed on Dec. 3, 2019.

(51) Int. Cl. C07K 14/635; A61P 19/02

(52) U.S. Cl. CPC C07K 14/635; A61P 19/02

(57) ABSTRACT

A method for treating low back pain (LBP) and/or osteoarthritic pain in a subject in need of treatment thereof, the method comprising administering to the subject a composition comprising a recombinant parathyroid hormone (PTH) and a pharmaceutically acceptable carrier is disclosed.

### CROSS-REFERENCE TO RELATED APPLICATIONS

This application claims the benefit of U.S. Provisional Patent Application No. 62/942,945, filed Dec. 3, 2019, the contents of which are incorporated by reference herein.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 5,918 Byte ASCII (Text) file named “38153-601_ST25,” created on Dec. 2, 2020.

### FEDERALLY SPONSORED RESEARCH OR DEVELOPMENT

This invention was made with Government support under AR071432 awarded by the National Institutes of Health (NIH). The Government has certain rights in the invention.

### BACKGROUND

Low back pain (LBP) is a common health problem, which most people (80%) experience at some point, especially in older adults. Rubin, 2007; Hartvigsen et al., 2006; Hartvigsen et al., 2004; and Global, regional, and national incidence, prevalence, and years lived with disability for 310 diseases and injuries, 1990-2015: a systematic analysis for the Global Burden of Disease Study 2015. In the United States alone, the direct and indirect costs associated with LBP surpass $90 billion per year, with similar adjusted rates in other countries. Samartzis and Grins, 2017. Ninety percent of LBP is nonspecific LBP, which has no apparent pathoanatomical cause. Krismer and van Tulder, 2007; Koes et al., 2006. Several lumbar structures, such as intervertebral disc, facet joints, are plausible sources of nonspecific LBP, but the pain cannot be reliably attributed to those structures by clinical tests. Hancock et al., 2007; Maher et al., 2017; and Hartvigsen et al., 2018. Importantly, intervertebral disc (IVD) degeneration is frequently observed in asymptomatic patients, indicating that disc degeneration, per se, is not painful in some patients. Hurri and Karppinen, 2004; Borenstein et al., 2001. Hence, identifying the source of LBP and related mechanisms is essential to develop effective treatments for LBP.

Further, osteoarthritis (OA) is a leading cause of disability as the most common degenerative joint disorder and chronic pain is the most prominent symptom of osteoarthritis (OA), affecting nearly 40 million people in the US. Pain itself also is a major risk factor for the development of future functional limitation and disability in OA patients. Unfortunately, OA pain treatment remains challenging and represents a large unmet medical need. It is not clear what causes OA pain, and currently there is no effective way to relieve it. Available therapies (NSAIDs, steroids, visco-supplementation, such as intra-articular injection of hyaluronic acid) only alleviate mild joint OA pain. Relief from chronic OA pain remains an unmet medical need and still major reason for seeking surgical intervention. Despite these efforts, the origins of pain and its molecular mechanisms remain poorly understood.

### SUMMARY

In some aspects, the presently disclosed subject matter provides a method for treating low back pain (LBP) and/or osteoarthritic pain in a subject in need of treatment thereof, the method comprising administering to the subject a composition comprising a recombinant parathyroid hormone (PTH) and a pharmaceutically acceptable carrier.

In certain aspects, the low back pain comprises a nonspecific low back pain.

In certain aspects, the administering of the recombinant parathyroid hormone (PTH) inhibits osteoclast activity-induced sensory innervation in a vertebral endplate of the subject.

In other aspects, the administering of the recombinant parathyroid hormone (PTH) increases the intervertebral disc (IVD) space by decreasing the volume and porosity of sclerotic endplates.

In yet other aspects, the administering of the recombinant parathyroid hormone (PTH) prevents endplate remodeling and sclerosis.

In even yet other aspects, the administering of the recombinant parathyroid hormone (PTH) reduces sensory nerve fibers.

In other aspects, the administering of the recombinant parathyroid hormone (PTH) reduces the porosity of sclerotic endplates.

In particular aspects, the administering of the recombinant parathyroid hormone (PTH) treats the osteoarthritic pain by one or more of inhibition of nerve innervation, inhibition of subchondral bone deterioration, inhibition of articular cartilage degeneration, attenuation of joint degeneration, decelerating subchondral bone deterioration, and sustaining of subchondral bone microarchitecture by remodeling.

In some aspects, the method further comprises administering at least one other agent in combination with the administering of the recombinant parathyroid hormone (PTH).

In certain aspects, the at least one other agent is selected from the group consisting of paracetamol, an opioid, a non-steroidal anti-inflammatory drug, a skeletal muscle relaxant, a triptan, an α2-agonist, a local anesthetic, a tricyclic antidepressant, a benzodiazepine, a steroid, a visco supplement, and combinations thereof.

In particular aspects, the low back pain is associated with one or more of spine degeneration, lumbar disc herniation (LDH), scoliosis, cancer, and an infection.

In some aspects, the recombinant PTH comprises a full-length PTH protein or a fragment of PTH. In particular aspects, the recombinant parathyroid hormone comprises teriparatide (PTH(1-34′)). In other aspects, the recombinant parathyroid hormone comprises an intact parathyroid hormone (iPTH).

In certain aspects, the composition is administered to the subject at least once a day.

In other aspects, the presently disclosed subject matter provides the use of a recombinant parathyroid hormone to treat low back pain (LBP) or osteoarthritic pain in a subject in need thereof.

Certain aspects of the presently disclosed subject matter having been stated hereinabove, which are addressed in whole or in part by the presently disclosed subject matter, other aspects will become evident as the description proceeds when taken in connection with the accompanying Examples and Drawings as best described herein below.

### BRIEF DESCRIPTION OF THE FIGURES

The patent or application file contains at least one drawing executed in color. Copies of this patent or patent application publication with color drawings will be provided by the Office upon request and payment of the necessary fee.

Having thus described the presently disclosed subject matter in general terms, reference will now be made to the accompanying Figures, which are not necessarily drawn to scale, and wherein:

FIG. 1-1A, FIG. 1-1B, FIG. 1-1C, FIG. 1-1D, FIG. 1-1E, FIG. 1-1F, FIG. 1-1G, FIG. 1-1H, FIG. 1-1I, FIG. 1-1J. FIG. 1-1K, FIG. 1-1L, and FIG. 1-1M show symptomatic spinal pain behavior in LSI model and aged mice. (FIG. 1-1A) Pressure hyperalgesia of the lumbar spine was assessed as the force threshold to induce the vocalization by a force gauge after LSI or sham surgery. (FIG. 1-1B-FIG. 1-1E) Spontaneous activity analysis including distance traveled (FIG. 1-1B), maximum speed (FIG. 1-1C), mean speed (FIG. 1-1D) and active time (FIG. 1-1E) on the wheel per 24 h, determined by the percentage of sham surgery mice at the corresponding time points. (FIG. 1-1F, FIG. 1-1G) The hind paw withdrawal frequency responding to mechanical stimulation (von Frey, 0.7 mN and 3.9 mN) after LSI or sham surgery. PWF: Paw Withdraw Frequency. *p<0.05, **p<0.01 compared with the sham surgery mice at the corresponding time points. n=6 per group (a-g). (FIG. 1-1H) Pressure hyperalgesia of the low back in 20-month-old or 3-month-old mice. (FIG. 1-1I) The distance traveled. (FIG. 1-1J) mean speed and (FIG. 1-1K) active time on the wheel per 24 h in 20-month-old mice determined by the percentage of 3-month-old mice. (FIG. 1-1L, FIG. 1-1M) The hind paw withdrawal frequency responding to mechanical stimulation (von Frey, 0.7 mN and 3.9 mN) in 20-month-old or 3-month-old mice. PWF: Paw Withdraw Frequency. *p<0.05, **p<0.01 compared with the 3-month-old mice. n=8 per group (h-m). Statistical significance was determined by multifactorial ANOVA, and all data are shown as means±standard deviations;

FIG. 1-2A, FIG. 1-2B, FIG. 1-2C, FIG. 1-2D, FIG. 1-2E, and FIG. 1-2F demonstrate that sensory innervation in endplates correlates with increase of osteoclasts in LSI model. (FIG. 1-2A) Representative images of coronal mouse caudal endplate sections of L4/5 stained for TRAP (magenta) at 2, 4 and 8 weeks after LSI or sham surgery. Scale bars, 50 μm. (FIG. 1-2B) Quantitative analysis of the number of TRAP⁺ cells in endplates. (FIG. 1-2C) Representative immunofluorescent images of CGRP⁺ sensory nerve fibers (red) and DAPI (blue) staining of nuclei in mouse caudal endplates of L4/5 at 2, 4 and 8 weeks after LSI or sham surgery. Scale bars, 50 μm. (FIG. 1-2D) Percentage of CGRP⁺ area in endplates. (FIG. 1-2E) Representative images of immunofluorescent analysis of CGRP⁺ (red), PGP9.5⁺ (green) nerve fibers and DAN (blue) staining of nuclei in mouse caudal endplates of L4/5 at 8 weeks after LSI or sham surgery. Scale bars, 50 μm. (FIG. 1-2F) Representative images of immunofluorescent analysis of IB4⁺ (green) sensory nerve fibers and DAPI (blue) staining of nuclei in mouse caudal endplates of L4/5 at 2, 4 and 8 weeks after LSI or sham surgery. Scale bars, 100 μm. **p<0.01 compared with the sham surgery mice at the corresponding time points. n=6 per group (FIG. 1-2B, FIG. 1-2D). Statistical significance was determined by multifactorial ANOVA, and all data are shown as means±standard deviations;

FIG. 1-3A, FIG. 1-3B, FIG. 1-3C, FIG. 1-3D, FIG. 1-3E, FIG. 1-3F, FIG. 1-3G, FIG. 1-3H, and FIG. 1-31 demonstrate that sensory innervation in endplates correlates with increase of osteoclasts during aging. (FIG. 1-3A) Representative three-dimensional high-resolution μCT images of the mouse caudal endplates of L4/5 (coronal view) in 3-month-old and 20-month-old mice. Scale bars, 1 mm. (FIG. 1-3B-FIG. 1-3C) Quantitative analysis of the total porosity (FIG. 1-3B) and trabecular separation (Tb. Sp; FIG. 1-3C) of the mouse caudal endplates of L4/5 determined by μCT. (FIG. 1-3D) Top and middle, representative images of safranin O and fast green staining of coronal caudal endplate sections of L4/5 in 3-month-old and 20-month-old mice, proteoglycan (red) and cavities (green). Bottom, representative images of TRAP (magenta) staining of coronal sections of the caudal endplates of L4/5 in 3-month-old and 20-month-old mice. Scale bars, 50 μm (FIG. 1-3E) Endplate scores in 3-month-old and 20-month-old mice as an indication of endplate degeneration based on safranin O and fast green staining. (FIG. 1-3F) Quantitative analysis of the number of TRAP⁺ cells in endplates. (FIG. 1-3G) Representative images of immunofluorescent analysis of CGRP⁺ sensory nerve fibers (red) and DAPI (blue) staining of nuclei in mouse caudal endplates of L4/5 in 20-month-old and 3-month-old mice. Scale bars, 50 μm. (FIG. 1-3H) Quantitative analysis of the percentage of CGRP⁺ area in endplates. (FIG. 1-3I) Representative images of immunofluorescent analysis of CGRP⁺ (green), PGP9.5⁺ (red) nerve fibers and DAPI (blue) staining of nuclei in mouse caudal endplates of L4/5 in 20-month-old mice. Scale bars, 50 μm. *p<0.05. **p<0.01 compared with the 3-month-old mice. n=6 per group (FIG. 1-3B, FIG. 1-3C, FIG. 1-3E, FIG. 1-3F, and FIG. 1-3H). Statistical significance was determined by two-tailed Student's t-test, and all data are shown as means±standard deviations:

FIG. 1-4A, FIG. 1-4B, FIG. 1-4C, FIG. 1-4D, FIG. 1-4E, FIG. 1-4F, FIG. 1-4G, FIG. 1-4H, and FIG. 1-4I demonstrate that sensory innervation in endplates is validated by retrograde and anterograde tracing. (FIG. 1-4A) Model of retrograde tracing of the sensory innervation in the endplates of L4/5. The T12-L6 DRGs were harvested at 1 week after injection of Dil in the left part of the mouse caudal endplates at 8 weeks after LSI or sham surgery. (FIG. 1-4B) Representative images of Dil⁺ (red) sensory neurons and DAPI (blue) staining of nuclei in the left (L) and right (R) side DRGs. Scale bars, 200 μm. (FIG. 1-4C) Quantitative analysis of the number of Dil cells of (b). **p<0.01 compared with the sham surgery mice at the corresponding side. n=6 per group. (FIG. 1-4D) Top, representative images of Dil⁺ (red) and CGRP⁺ sensory neurons and DAPI (blue) staining of nuclei in the left (L) side DRGs of L1 and L2. Bottom, representative images of Dil⁺ (red) and IB4⁺ sensory neurons and DAPI (blue) staining of nuclei in the left (L) side DRGs of L1 and L2. Scale bars, 100 μm. (FIG. 1-4E) Quantitative analysis of (d). **p<0.01 compared with the percentage of Dil^{+IB}4⁺ cells to Dil⁺ cells in the corresponding DRG. n=6 per group. (FIG. 1-4F-FIG. 1-4H) The anterograde tracing analysis of L1 and L2 DRG neuronal fibers innervation into the caudal endplates of L4/5. The Dil was injected in the L1 and L2 DRGs at 8 weeks after LSI and sham surgery; or in 3-month-old and 20-month-old mice. Representative images of Dil⁺ (red) sensory nerve fibers in the caudal endplates of L4/5 in LSI and sham surgery mice (FIG. 1-4F) or in 3-month-old and 20-month-old group (FIG. 1-4H) at 1 week after injection. Scale bars, 100 μm. Quantitative analysis of the percentage of Dil⁺ area in endplates in LSI and sham surgery group (FIG. 1-4G) or in 3-month-old and 20-month-old group (FIG. 1-4I). **p<0.01 compared with the 3-month-old mice. n=6 per group (FIG. 1-4G and FIG. 1-4I). Statistical significance was determined by multifactorial ANOVA, and all data are shown as means f standard deviations;

FIG. 1-5A, FIG. 1-5B, FIG. 1-5C, FIG. 1-5D, FIG. 1-5E, FIG. 1-5F, FIG. 1-5G, FIG. 1-5H, and FIG. 1-5I demonstrate that PGE2/EP4 contributes to the spinal pain hypersensitivity. (FIG. 1-54) Quantitative analysis of the expression of PGE synthetase (PGES), cox2, IL-1β, IL-17, IL-2 and TNF-α in lumbar endplates at 4 weeks after LSI determined by qRT-PCR_(FIG. 1-5B) Representative images of Immunohistochemical analysis of Cox2 (brown; top) or PGE2 (brown; bottom) in the caudal endplates of L4/5 at 4 and 8 weeks after LSI or sham surgery. Scale bars, 50 μm. (FIG. 1-5C) ELISA analysis of PGE2 concentration in the lysate of lumbar endplates at 4.8 and 12 weeks after LSI surgery. *p<0.05, **p<0.01 compared with the sham surgery mice. n=3 (FIG. 1-5A and FIG. 1-5C). (FIG. 1-5D) Representative images of immunofluorescent analysis of CGRP (red), EP4 (green) staining and DAPI (blue) staining of nuclei in the caudal endplates of L4/5 at 4 and 8 weeks after LSI surgery. Scale bars, 50 μm. (FIG. 1-5E) Representative images of immunofluorescent analysis of CGRP (red), EP4 (green) staining and DAPI (blue) staining of nuclei in the L2 DRGs at 4 and 8 weeks after LSI surgery. Scale bars, 100 μm. (FIG. 1-5F) Quantitative analysis of percentage of CGRP⁺ EP4⁺ cells to CGRP cells in the L2 DRGs at 4 and 8 weeks after LSI surgery. (FIG. 1-5G) Representative images of immunofluorescent analysis of CGRP (red), Naᵥ 1.8 (green) staining and DAPI (blue) staining of nuclei in the caudal endplates of L4/5 at 4 weeks after LSI surgery. Scale bars, 50 μm. (FIG. 1-5H) Representative images of immunofluorescent analysis of CGRP (red). Naᵥ 1.8 (green) staining and DAPI (blue) staining of nuclei in the L2 DRGs at 4 and 8 weeks after LSI surgery. Scale bars, 100 μm. (FIG. 1-5I) Quantitative analysis of percentage of CGRP⁺ Naᵥ 1.8⁺ cells to CGRP⁺ cells in the L2 DRGs at 4 and 8 weeks after LSI surgery. **p<0.01 compared with the sham surgery mice at the corresponding time points. n=6 per group (FIG. 1-5F and FIG. 1-5I). Statistical significance was determined by multifactorial ANOVA, and all data are shown as means f standard deviations;

FIG. 1-6A, FIG. 1-6B, FIG. 1-6C, FIG. 1-6D, FIG. 1-6E, FIG. 1-6F, FIG. 1-6G, FIG. 1-6H, FIG. 1-6I, FIG. 1-6J, and FIG. 1-6K demonstrates that PGE2 stimulates PKA/CREB signaling through EP4 to induce sodium influx. (FIG. 1-6A) Representative images of sodium indicator (green) analysis pre- and post-PGE2 (20 μM) stimulation for 5 min in primary DRG neurons from EP4^{f/f} for EP4^{-/-} mice, indicating sodium influx. Scale bar, 100 μm. Magnification, scale bar, 20 μm. (FIG. 1-6B, FIG. 1-6C) Quantitative analysis of the fluorescent density distribution of the 1^{st} (FIG. 1-6B) and 2^{nd} (FIG. 1-6C) column in (FIG. 1-6A). *p<0.05, **p<0.01 compared with the corresponding pre-treatment group. n=3 per group. (FIG. 1-6D) Western blots of the phosphorylation of PKA and CREB in primary DRG neurons treated with PGE2 (20 LM) for 30 min and PKA inhibitor (H-89, 10 μM) for 60 min. (FIG. 1-6E) Quantitative analysis of (FIG. 1-6D). **p<0.01 compared with the negative control group from EP4^{f/f} mice. ^{#}p<0.05, ^{#}p<0.01 compared with only PGE2 treatment group from EP4^{f/f} mice n=3 per group. (FIG. 1-6F) 1^{st} to 3^{rd} row, representative images of immunofluorescent analysis of PKA (red), p-PKA (green) staining and DAPI (blue) staining of nuclei: 4^{th} to 6^{th} row, representative images of immunofluorescent analysis of CREB (red), p-CREB(green) staining and DAPI (blue) staining of nuclei pre- and post-PGE2 (20 μM) stimulation combined with H-89 (10 μM) in primary DRG neurons from EP4^{f/f} or EP4^{-/-} mice. Scale bar, 100 μm. (FIG. 1-6G) Representative images of sodium indicator (green) analysis pre- and post-PGE2 (20 μM) stimulation combined with cAMP, PKA inhibitor (H-89) or siRNA for Naᵥ 1.8 (si-Naᵥ1.8) in primary DRG neurons from EP4^{f/f} for EP4^{-/-} mice. Scale bar, 100 μm. Magnification, scale bar, 20 μm. (FIG. 1-6H-FIG. 1-6K) Quantitative analysis of the fluorescent density distribution of the 1^{st} (FIG. 1-6H), 2^{nd} (FIG. 1-6I), 3^{rd} (FIG. 1-6J), 4^{th} (FIG. 1-6K) column in (FIG. 1-6G). *p<0.05, **p<0.01 compared with the corresponding pre-treatment group. n=3 per group. Statistical significance was determined by multifactorial ANOVA, and all data are shown as means t standard deviations;

FIG. 1-7A. FIG. 1-7B. FIG. 1-7C, FIG. 1-7D, FIG. 1-7E, FIG. 1-7F, and FIG. 1-7G demonstrate that EP4 knockout in sensory nerve attenuates spinal pain behavior. (FIG. 1-7A-FIG. 1-7G) Quantitative analysis of spinal pain-related behavior tests, including pressure hyperalgesia (FIG. 1-7A), spontaneous distance travelled (FIG. 1-7B), maximum speed (FIG. 1-7C), mean speed (FIG. 1-7D), active time (FIG. 1-7E) per 24 hours and hind paw withdrawal frequency responding to mechanical stimulation (0.7 mN: FIG. 1-7F and 3.9 mN; FIG. 1-7G) in EP4 or EP4^{f/f} mice overtime after LSI or sham surgery. PWF: Paw withdraw frequency. *p<0.05. **p<0.01 compared with EP4^{f/f} sham surgery mice, ^{#}p<0.05, ^{##}p<0.01 compared with EP4^{f/f} LSI surgery mice at the corresponding time points. n=8 per group (FIG. 1-7A-FIG. 1-7G). Statistical significance was determined by multifactorial ANOVA, and all data are shown as mean±standard deviations;

FIG. 1-8A, FIG. 1-8B, FIG. 1-8C, FIG. 1-8D, FIG. 1-8E, FIG. 1-8F, FIG. 1-8G, FIG. 1-8H. FIG. 1-81, FIG. 1-8J, FIG. 1-8K. FIG. 1-8L, FIG. 1-8M, FIG. 1-8N, FIG. 1-8O, and FIG. 1-8P show that decreased osteoclasts activity diminishes sensory innervation and attenuates pain. (FIG. 1-8A) Representative μCT images of the caudal endplates of L4/5 (coronal view) in Rankl^{-/-} or Rankl^{f/f} mice at 4 and 8 weeks after LSI or sham surgery. Scale bars, 1 mm. (FIG. 1-8B-FIG. 1-8C) Quantitative analysis of the total porosity (FIG. 1-8B) and trabecular separation (Th. Sp; FIG. 1-8C) of the mouse caudal endplates of L4/5 determined by μCT. (FIG. 1-8D) Representative images of safranin O and fast green staining of coronal sections of the caudal endplates of L4/5 in Rankl^{-/-} or Rankl^{f/f} mice at 4 and 8 weeks after LSI or sham surgery. Scale bars, 50 μm. (FIG. 1-8E) Endplate scores of the caudal endplates. (FIG. 1-8F) Representative images of TRAP (magenta) staining of coronal sections of the caudal endplates of L4/5 in Rankl^{-/-} or Rankl^{f/f} mice at 4 and 8 weeks after LSI or sham surgery. Scale bars, 50 μm. (FIG. 1-8G) Quantitative analysis of the number of TRAP⁺ cells in the caudal endplates. (FIG. 1-8H) Representative images of immunofluorescent analysis of CGRP⁺ sensory nerve fibers (red) and DAPI (blue) staining of nuclei in caudal endplates of L4/5 in Rankl^{-/-} or Rankl^{f/f} mice at 4 and 8 weeks after LSI or sham surgery. Scale bars, 50 μm. (FIG. 1-8I) Quantitative analysis of the percentage of CGRP⁺ area in caudal endplates. (FIG. 1-8J-FIG. 1-8P) Quantitative analysis of spine pain-related behavior tests, including pressure hyperalgesia (FIG. 1-8J), distance travelled (FIG. 1-8K), maximum speed (FIG. 1-8L), mean speed (FIG. 1-8M), active time (FIG. 1-8N) per 24 hours and hind paw withdrawal frequency responding to mechanical stimulation (0.7 mN; FIG. 1-8O and 3.9 mN; FIG. 1-8P) in Rankl^{-/-} or Rankl^{f/f} mice overtime after LSI or sham surgery. **p<0.01 compared with Rankl^{f/f} sham surgery mice, **p<0.01 compared with Rankl^{f/f} LSI surgery mice at the corresponding time points. n=8 per group (FIG. 1-8B. FIG. 1-8C, FIG. 1-8E, FIG. 1-8G, and FIG. 1-81-FIG. 1-8P). Statistical significance was determined by multifactorial ANOVA, and all data are shown as means±standard deviations;

FIG. 1-9A, FIG. 1-9B, FIG. 1-9C, FIG. 1-9D, FIG. 1-9E, FIG. 1-9F, FIG. 1-9G, FIG. 1-9H, FIG. 1-91, FIG. 1-9J, FIG. 1-9K, FIG. 1-9L, FIG. 1-9M, FIG. 1-9N, FIG. 1-9O, and FIG. 1-9P demonstrate that knockout of netrin-1 abrogates sensory innervation and spinal pain. (FIG. 1-9A) Representative images of immunofluorescent analysis of TRAP⁺ (red), Netrin-1⁺ (green) and DAPI (blue) staining of nuclei in caudal endplates of L4/5 after LSI or sham surgery. Scale bars, 50 m. (FIG. 1-9B) ELISA analysis of Netrin-1 concentration in the lysate of lumbar endplates after LSI surgery. **p<0.01 compared with the sham surgery mice. n=3 per group. (FIG. 1-9C) Representative images of immunofluorescent analysis of CGRP⁺ (red), DCC⁺ (green) and DAPI (blue) staining of nuclei in caudal endplates of L4/5 after LSI surgery. Scale bars, 50 μm. (FIG. 1-9D) Representative images of safranin O and fast green staining of the caudal endplates of L4/5 in Netrin-1^{-/-} or Netrin-1^{f/f} mice after LSI or sham surgery. Scale bars, 50 μm. (FIG. 1-9E) Endplate scores of the caudal endplates. (FIG. 1-9F) Representative images of TRAP (magenta) staining of the caudal endplates of L4/5 in Netrin-1^{-/-} or Netrin-1^{f/f} mice after LSI or sham surgery. Scale bars, 50 μm. (FIG. 1-9G) Quantitative analysis of (FIG. 1-9F). (FIG. 1-9H) Representative images of immunofluorescent analysis of CGRP⁺ (red) and DAPI (blue) staining of nuclei in caudal endplates of L4/5 in Netrin-1^{-/-} or Netrin-1^{f/f} mice after LSI or sham surgery. Scale bars, 50 μm. (FIG. 1-9I) Quantitative analysis of (FIG. 1-9H). (FIG. 1-9J-FIG. 1-9P) Quantitative analysis of spinal pain-related behavior tests, including pressure hypersensitivity (FIG. 1-9J), distance travelled (FIG. 1-9K), maximum speed (FIG. 1-9L), mean speed (FIG. 1-9M), active time (FIG. 1-9N) per 24 hours and hind paw withdrawal frequency responding to mechanical stimulation (0.7 mN; FIG. 1-9O and 3.9 mN; FIG. 1-9P) in Netrin-1^{-/-} or Netrin-1^{f/f} mice after LSI or sham surgery. *p<0.05. **p<0.01 compared with Netrin-If sham surgery mice, ^{#}p<0.05, ^{#}p<0.01 compared with Netrin-1^{f/f} LSI surgery mice at the corresponding time points, ns, no significant difference, compared with Netrin-If LSI surgery mice at the corresponding time points. n=7 per group (FIG. 1-9G, FIG. 1-91-FIG. 1-9P). Statistical significance was determined by multifactorial ANOVA, and all data are shown as means f standard deviations:

FIG. 1-10A and FIG. 1-10B show the formation of CD31⁺Emcn⁺ vessels in Endplate during spinal instability. (FIG. 1-10A) Representative images of immunofluorescent analysis of CD31⁺ (green). Emcn⁺ (red) and DAPI (blue) staining of nuclei in mouse caudal endplates of L4/5 at 2, 4 and 8 weeks after LSI or sham surgery. Scale bars, 50 μm. (FIG. 1-10B) Percentage of CD31⁺Emcn⁺ area in endplates. **p<0.01 compared with the sham surgery mice at the corresponding time points. n=6 per group. Statistical significance was determined by multifactorial ANOVA, and all data are shown as means±standard deviations;

FIG. 1-11A, FIG. 1-11B, FIG. 1-11C, FIG. 1-11D, and FIG. 1-11E show the μCT analysis of the vertebral trabecular bone during aging. (FIG. 1-11A) Representative three-dimensional high-resolution μCT images of the trabecular bone of L5 vertebrae (coronal view) in 3-month-old and 20-month-old mice. Scale bars, 1 mm. (FIG. 1-11B-FIG. 1-11E) Quantitative analysis of the trabecular bone volume/total volume (BV/TV) (FIG. 1-11B) and trabecular bone number (Tb.N, FIG. 1-11C), trabecular bone thickness (Tb.Th, FIG. 1-11D), and trabecular bone separation distribution (Th. Sp, FIG. 1-11E) in L5 vertebrae determined by μCT. **p<0.01, ns, not significant difference compared with the 3-month-old mice. n=6 per group. Statistical significance was determined by two-tailed Student's t test, and all data are shown as mean±standard deviations:

FIG. 1-12A and FIG. 1-12B show the formation of CD31⁺Emcn⁺ vessels in Endplate during aging. (FIG. 1-12A) Representative images of immunofluorescent analysis of CD31⁺ (green), Emcn⁺ (red) and DAPI (blue) staining of nuclei in mouse caudal endplates of L4/5 in 20-month-old and 3-month-old mice. Scale bars, 50 μm. (FIG. 1-12B) Quantitative analysis of the percentage of CD31⁺Emcn⁺ area in endplates. **p<0.01 compared with the 3-month-old mice. n=6 per group. Statistical significance was determined by two-tailed Student's t test, and all data are shown as means±standard deviations;

FIG. 1-13A, FIG. 1-13B, FIG. 1-13C, and FIG. 1-13D demonstrate nerve innervation in the human sclerotic endplates. (FIG. 1-13A) Representative gross appearance (top) and images of safranin O and fast green staining of coronal sections (bottom) of the endplates from patients without LBP or with LBP. Scale bars, 50 μm. (FIG. 1-13B) Endplate scores of the samples from patients without LBP or with LBP. (FIG. 1-13C) Representative images of TRAP (magenta) staining of coronal sections of the endplates from patients without LBP or with LBP. Scale bars, 50 μm. (FIG. 1-13D) Representative immunofluorescent images of CGRP⁺ (red), PGP9.5⁺ (green) and DAPI (blue) staining of nuclei in the endplates. Scale bars, 50 μm. **p<0.01 compared with patients without LBP. n=6 of non-LBP group, n=9 of LBP group. LBP: low back pain. Statistical significance was determined by two-tailed Student's t test, and all data are shown as means±standard deviations;

FIG. 1-14 shows the potential source of PGE2 in porous endplates. Representative immunofluorescent images of cox2⁺ (red) and F4/80⁺ (green). cox2⁺ (red) and OCN⁺ (green), cox2⁺ (red) and TRAP⁺ (green) and DAPI (blue) staining of nuclei in the endplates. Scale bars, 50 μm;

FIG. 1-15A and FIG. 1-15B show that EP4 knockout did not affect the LSI-induced increase in the number of TRAP⁺ osteoclasts in endplates. (FIG. 1-15A) Representative images of coronal mouse caudal endplate sections of L4/5 stained for TRAP (magenta) at 8 weeks after LSI or sham surgery in EP4^{f/f} and EP4^{-/-} mice. Scale bars, 50 μm. (FIG. 1-15B) Quantitative analysis of the number of TRAP⁺ cells in endplates. **p<0.01 compared with sham surgery group. ns, not significant difference compared with sham surgery mice in corresponding transgenic group. n=6 per group. Statistical significance was determined by multifactorial ANOVA, and all data are shown as means±standard deviations;

FIG. 1-16A, FIG. 1-16B, FIG. 1-16C, FIG. 1-16D, and FIG. 1-16E show the osteopetrotic phenotype of Vertebrae in Rankl^{-/-} mice. (FIG. 1-16A) Representative μCT images of the trabecular bone (coronal view) in L5 vertebrae of Rankl^{-/-} and Rankl^{f/f} mice in sham or LSI surgery group. Scale bars, 1 mm. (FIG. 1-16B-FIG. 1-16E) Quantitative analysis of the Trabecular BV/TV (FIG. 1-16B), Tb.N (FIG. 1-16C), Tb.Th (FIG. 1-16D), and Tb. Sp (FIG. 1-16E) of the mouse L5 vertebrae determined by μCT. BV, Bone Volume. TV, Total Volume. Tb.N, Trabecular bone Number. Tb.Th, Trabecular bone Thickness. Tb. Sp, Trabecular Separation distribution. **p<0.01 compared with Rankl^{f/f} mice. ns, no significant difference compared with sham surgery mice in corresponding transgenic group. n=6 per group. Statistical significance was determined by multifactorial ANOVA, and all data are shown as means±standard deviations;

FIG. 1-17A and FIG. 1-17B demonstrate that decreased osteoclasts activity diminished the formation of CD31⁺Emcn⁺ vessels in Endplate. (FIG. 1-17A) Representative images of immunofluorescent analysis of CD31⁺ (green), Emcn⁺ (red) and DAPI (blue) staining of nuclei in caudal endplates of L4/5 in Rankl^{-/-} or Rankl^{f/f} mice at 4 and 8 weeks after LSI or sham surgery. Scale bars, 50 μm. (FIG. 1-17B) Quantitative analysis of the percentage of CD31⁺Emcn⁺ area in caudal endplates. **p<(0.01 compared with Rankl^{f/f} sham surgery mice, ^{##}p<0.01 compared with Rankl^{f/f} LSI surgery mice at the corresponding time points n=8 per group. Statistical significance was determined by multifactorial ANOVA, and all data are shown as means f standard deviations;

FIG. 1-18A and FIG. 1-18B demonstrate that reduction of osteoclast activity did not inhibit sensory innervation in the annulus fibrosus in LSI mice. (FIG. 1-18A) Representative immunofluorescent images of CGRP⁺ sensory nerve fibers (red) and DAPI (blue) staining of nuclei in mouse annulus fibrosus of L4/5 at 8 weeks after LSI or sham surgery. Scale bars, 100 μm. (FIG. 1-18B) Percentage of CGRP⁺ area in annulus fibrosus. **p<0.01 compared with sham surgery mice, ns, not significant difference, compared with Rankl^{f/f} LSI surgery mice. n=6 per group. Statistical significance was determined by multifactorial ANOVA, and all data are shown as means±standard deviations;

FIG. 1-19A and FIG. 1-19B demonstrate that knockout of netrin-1 in the TRAP+ lineage cells inhibited the formation of CD31⁺Emcn⁺ vessels in Endplate. (FIG. 1-19A) Representative images of immunofluorescent analysis of CD31⁺ (green), Emcn⁺ (red) and DAPI (blue) staining of nuclei in caudal endplates of L4/5 in Netrin-1^{-/-} or Netrin-1^{f/f} mice at 4 and 8 weeks after LSI or sham surgery. Scale bars, 50 μm. (FIG. 1-19B) Quantitative analysis of the percentage of CD31⁺Emcn⁺ area in caudal endplate. **p<0.01 compared with Netrin-1^{f/f} sham surgery mice, ^{##}p<0.01 compared with Netrin-1^{f/f} LSI surgery mice at the corresponding time points. n=7 per group. Statistical significance was determined by multifactorial ANOVA, and all data are shown as means±standard deviations;

FIG. 1-20A and FIG. 1-20B demonstrate that knockout of Netrin-1 in the Trap⁺ cells did not inhibit sensory innervation in the annulus fibrosus in LSI mice. (FIG. 1-20A) Representative immunofluorescent images of CGRP⁺ sensory nerve fibers (red) and DAPI (blue) staining of nuclei in mouse annulus fibrosus of L4/5 at 8 weeks after LSI or sham surgery. Scale bars, 100 μm. (FIG. 1-20B) Percentage of CGRP⁺ area in annulus fibrosus. **p<0.01 compared with sham surgery mice, ns, not significant difference, compared with Netrin-1^{f/f} LSI surgery mice. n=6 per group. Statistical significance was determined by multifactorial ANOVA, and all data are shown as means±standard deviations:

FIG. 2-1A, FIG. 2-1B, FIG. 2-1C, FIG. 2-1D, FIG. 2-1E, FIG. 2-1F, FIG. 2-1G, FIG. 2-1H, FIG. 2-1I, and FIG. 2-1J demonstrate that iPTH attenuated low back pain related behavior test in LSI mouse model and Aging Mice. (FIG. 2-1A) Lumbar spine instability mouse model (LSI) and Aging mouse (FIG. 2-1F) had been done the surgery or treatment according to the schedule (FIG. 2-1B, FIG. 2-1G) Pressure hyperalgesia of the lumbar spine assessed as the force threshold to induce the vocalization by a force gauge in LSI mouse model (FIG. 2-1B) and Aging model (FIG. 2-1G). (FIG. 2-1C-FIG. 2-1E, FIG. 2-1H-FIG. 2-1J) Spontaneous activity analysis including active time (FIG. 2-1C, FIG. 2-1H), distance traveled (FIG. 2-1D, FIG. 2-1I) and mean speed (FIG. 2-1E, FIG. 2-1J) on the wheel per 24 h in Sham, iPTH or vehicle treatment group. *p<0.05, **p<0.01 compared with the sham surgery mice or vehicle group at the corresponding time points. n=8 per group. Statistical significance was determined by multifactorial ANOVA or T-test, and all data are shown as means±standard deviation;

FIG. 2-1A, FIG. 2-1B, FIG. 2-1C, FIG. 2-1D, FIG. 2-IE, FIG. 2-1F, FIG. 2-1G, FIG. 2-1H, FIG. 2-1I, and FIG. 2-1J show that iPTH attenuated low back pain related behavior test in LSI mouse model and Aging Mice (FIG. 2-1A) Lumbar spine instability mouse model (LSI) and (FIG. 2-1F) Aging mouse had been done the surgery or treatment according to the schedule. (FIG. 2-1B-FIG. 2-1J) Spontaneous activity analysis including distance traveled (FIG. 2-1B, FIG. 2-1G), mean speed (FIG. 2-1C, FIG. 2-1H) and active time (FIG. 2-1D, FIG. 2-1I) on the wheel per 24 h in Sham, iPTH or vehicle treatment group. (FIG. 2-1E, FIG. 2-1J) Pressure hyperalgesia of the lumbar spine assessed as the force threshold to induce the vocalization by a force gauge in LSI mouse model (FIG. 2-1E) and Aging model (FIG. 2-1J). *p<0.05, **p<0.01 compared with the sham surgery mice or vehicle group at the corresponding time points. n=8 per group. Statistical significance was determined by multifactorial ANOVA or T-test, and all data are shown as means±standard deviations;

FIG. 2-2A, FIG. 2-2B, FIG. 2-2C, FIG. 2-2D, FIG. 2-2E, FIG. 2-2F, FIG. 2-2G, FIG. 2-2H demonstrate that PTH increased the IVD space by decreasing the volume and porosity of sclerotic endplates. (FIG. 2-2A) (Top) Representative coronal high-resolution microcomputed tomography (μCT) images and (Bottom) three-dimensional images of the L4/5 mouse endplates in Sham surgery group, LSI mice treated with vehicle or iPTH groups, and Aging mice treated with vehicle or iPTH groups. Scale bars, 0.5 mm. (FIG. 2-2B) Representative images of safranin O and fast green staining of coronal endplate sections of L4/5 in Sham, LSI treated with vehicle or PTH, Aging treated with vehicle or PTH groups, proteoglycan (red) and cavities (green). Scale bars, 100 m. (FIG. 2-2C-FIG. 2-2E) Quantitative analysis of the total porosity (FIG. 2-2C), endplate volume (FIG. 2-2D) and volume of porosity (FIG. 2-2E) of the mouse L4/5 endplate. (FIG. 2-2F) Quantitative analysis of the area of cartilage in the endplate based on safranin O and fast green staining as an indication of cartilage endplate degeneration. (FIG. 2-2G-FIG. 2-2H) Quantification of IVD height and endplate thickness in the back 1/3 of L4/L5 sagittal plane. n=8 per group. Data are shown as mean±s.d. *p<0.05. **p<0.01;

FIG. 2-3A, FIG. 2-3B, FIG. 2-3C, FIG. 2-3D, FIG. 2-3E, FIG. 2-3F, FIG. 2-3G, FIG. 2-3H demonstrate that sensory innervation decreased in PTH remodeling of sclerotic endplates. (FIG. 2-3A-FIG. 2-3B) Representative images of immunofluorescent analysis of PGP9.5+(green) nerve fibers and DAPI (blue) staining of nucleus in mouse endplates (B 1.2) and annular fibrosis (B 3) of L4/5 of Sham groups, LSI treated with Vehicle or PTH groups, and Aging treated with Vehicle or PTH groups Scale bars, 50 μm. (FIG. 2-3C-FIG. 2-3D) Quantitative analysis of the percentage of PGP9.5+ area in endplates (FIG. 2-3C) or annular fibrosis (FIG. 2-3D). (FIG. 2-3E-FIG. 2-3G) Retrograde tracing of the sensory innervation in the endplates of L4/5 with DIL and quantitative analysis of L1-2 DRG immuno-stained different sensory nerve fibers. Representative images of Dil+(red) sensory neuron of L1-2 DRG sections, DAPI (blue) staining of nuclei and different sensory nerve fiber markers (green) including PGP9.5, CGRP, IB4, P2X3, PIEZO2 or NF200 respectively (FIG. 2-3E), Scale bars, 100 μm. Quantitative analysis of the number of Dil+ cells of PGP9.5+ Cells (FIG. 2-3F) in Sham groups, LSI treated with Vehicle or PTH groups, and Aging treated with Vehicle or PH groups. Quantitative analysis of the percentage of Dil+NF200+. Dil+PIZO2+ or Dil+CGRP+ cells to Dil+ cells in the corresponding DRG. n=5 per group (FIG. 2-3G). Statistical significance was determined by multifactorial ANOVA, and all data are shown as means±standard deviations. *p<0.05, **p<0.01;

FIG. 2-4A, FIG. 2-4B, FIG. 2-4C, FIG. 2-4D, FIG. 2-4E, FIG. 2-4F, FIG. 2-4G, FIG. 2-4H, and FIG. 2-4I demonstrate that bone remodeling reduces the porosity of sclerotic endplate after iPTH (FIG. 2-4A-FIG. 2-4C) Representative images of TRAP (Red) staining (FIG. 2-4A), immunohistology of Osteocalcin (FIG. 2-4B), immunofluorescent analysis of CD31+ (green), Emcn+ (red) and DAPI (blue) staining (FIG. 2-4C) of coronal sections of the endplates of L4/5 in Sham, LSI treated with Vehicle or PTH groups, Aging treated with Vehicle or PTH groups. Scale bars, 50 m. (FIG. 2-4D-FIG. 2-4E) Representative images of double labeling with Calcein/Alizarin red (FIG. 2-4D), and Goldner staining (FIG. 2-4E) in the L4/5 endplate of Sham, LSI treated with Vehicle or PTH groups, Aging treated with Vehicle or PH groups. Scale bars, 50 m. (FIG. 2-4F-FIG. 2-4H) Quantitative analysis of the number of TRAP+ cells (FIG. 2-4F), the number of osteocalcin+ cells (FIG. 2-4G), the percentage of CD31+Emcn+ area in endplates (FIG. 2-4H). (FIG. 2-4I) Axon attractive or repulsive factors including netrin-1, sema3a, slit1, sli2, slit3, NGF, and the inflammatory factors including COX-2,PGES, IL-1, TNF-α expression in lumbar endplates at LSI with vehicle or PTH treatment determined by qRT-PCR. Data are shown as mean±s.d. *p<0.05. **p<0.01;

FIG. 2-5A, FIG. 2-5B, FIG. 2-5C, FIG. 2-5D, FIG. 2-5E, and FIG. 2-5F demonstrates that lower porosity endplate caused by iPTH was better to support the mechanical stress, which resulted in lower expression of COX-2 and PGE2. (FIG. 2-5A-FIG. 2-5C) Representative images of Finite element analysis including Miser stress and U magnitude (FIG. 2-5A), and immunohistochemical analysis of COX-2 (brown; Top) or PGE2 (brown: Bottom) in L4/5 upper endplates of sham group. LSI treated with Vehicle or PTH groups, and Aging treated with Vehicle or PTH groups. Scale bars, 50 μm. (FIG. 2-5C-FIG. 2-5F) Quantitative analysis of the Mister stress (FIG. 2-5C), the percentage of COX-2+ cells (FIG. 2-5D) or PGE2+ cells (FIG. 2-5E) in mouse L4/5 endplates. (FIG. 2-5F) ELISA analysis of PGE2 concentration in lysate of lumbar endplates. *p<0.05, **p<0.01 n=3 per group, statistical significance was determined by multifactorial ANOVA, and all data are shown as mean±s.d:

FIG. 2-6A, FIG. 2-6B, FIG. 2-6C, FIG. 2-6D, FIG. 2-6E, FIG. 2-6F, FIG. 2-6G, FIG. 2-6H, FIG. 2-61, FIG. 2-6J, FIG. 2-6K, and FIG. 2-6L demonstrate that iPTH attenuates endplate sclerosis and disc degeneration in aging monkey. (FIG. 2-6A-FIG. 2-6B) Representative T2 weighted MR images of same monkey in each group with vehicle (Top) or PTH (Bottom) treated 0 months, 3 months, and 6 months (FIG. 2-6A). Quantitative analysis of the change of Pfirrmann grade of same segment at same monkey with vehicle or PTH treated 0 months, 3 months, and 6 months (FIG. 2-6B). (FIG. 2-6C-FIG. 2-6E) Representative images of measuring T1ρ or T2 map value in nucleus pulposus (FIG. 2-6C). Quantitative analysis of T1ρ value (FIG. 2-6D) or T2 map value (FIG. 2-6E) in nucleus pulposus of the same monkey in each group. (FIG. 2-6F-FIG. 2-6L) Representative sagittal high-resolution microcomputed tomography (μCT) images (FIG. 2-6F, left), safranin O/fast green staining in low magnification and high magnification images (FIG. 2-6F, middle), CGRP and COX-2 immuno-staining (FIG. 2-6F, right) of the L4/5 monkeys' endplates in Vehicle group (Top) or PTH group (Bottom). Scale bars, 200 μm. (FIG. 2-6G-FIG. 2-6J) Quantitative analysis of the percentage of the total porosity (FIG. 2-6G), the endplate volume (FIG. 2-6H), the volume of porosity (FIG. 2-6I) and area of cartilage (FIG. 2-6K) in endplates. (FIG. 2-6K-FIG. 2-6L) Quantitative analysis of the percentage of CGRP nerve fibers (FIG. 2-6K) and COX-2+ cells (FIG. 2-6L) in the endplates of Vehicle group or PTH group. Data are shown as mean±s.d. *p<0.05, **p<0.01;

FIG. 2-7A, FIG. 2-7B, FIG. 2-7C, FIG. 2-7D, FIG. 2-7E, and FIG. 2-7F provide the information of aging monkey recruited in this study. (FIG. 2-7A) Aging rhesus monkeys had been screened by MRI and done the treatment according to the schedule. (FIG. 2-7B) Representative images of Pfirrmann grade 1-5 of nucleus pulposus in monkey. (FIG. 2-7C) The information of number, gender, and weight in recruited aging rhesus monkeys. (FIG. 2-7D) Blood (Serum) test including PINP, β-CTx, et al. in monkeys before PTH or Vehicle treatment. (FIG. 2-7E) The weight change of aging rhesus monkeys with PTH or Vehicle treatment. (FIG. 2-7F) The serum test including PINP, β-CTx, Phosphorus, ALP, Osterix, Calcium before intervention and after 3 m, 6 m PTH or vehicle treatment. Data are shown as mean±s.d. *p<0.05, **p<0.01. PINP: procollagen type 1 N propeptide; β-CTx: C-terminal cross-linked telopeptide of type I collagen; P: Phosphorus; ALP: Alkaline phosphatase: OST: Osterix; Ca: Calcium;

FIG. 3-1A, FIG. 3-1B, FIG. 3-4C, FIG. 3-1D, FIG. 3-4E, FIG. 3-1F, FIG. 3-1G, FIG. 3-1H, FIG. 3-1I, and FIG. 3-1J demonstrate that PTH improves OA pain and joint degeneration after DMM. (FIG. 3-1A) Paw withdrawal threshold (PWT) was tested at the left hind paw of sham-operated. PTH-treated DMM and vehicle-treated DMM mice at different time point. n=8/group. (FIG. 3-1B) Paw withdrawal threshold was tested by Pressure application measurement (PAM) device at the left knee joint of sham-operated, PTH-treated DMM and vehicle-treated DMM mice. n=8/group. (FIG. 3-4C) Representative images of gait analysis of sham-operated. PTH-treated DMM and vehicle-treated DMM mice. RH=right hind (pink). LH=left hind (green). RF=right front (blue), LF=left front (yellow). (FIG. 3-1D) Quantitative analysis of percentage LH paw intensity, LH area and LH swing speed relative to RH at 8 weeks after DMM. n=8/group. (FIG. 3-1E) Safranin O-Fast green staining of sagittal sections of tibia medial compartment, proteoglycan (red) and bone (green). Scale Bar, 500 μm. (FIG. 3-1F) OARSI scores at 2, 4- and 8-weeks post-surgery. n=8/group. (FIG. 3-1G, FIG. 3-4H) Immunohistochemical analysis of matrix metalloproteinase 13⁺ (MMP13, brown) and type X collagen⁺ (Col X, brown) in articular cartilage. scale Bar, 50 sm. (FIG. 3-1I. FIG. 3-1J) Quantitative analysis of MMP13⁺ and Col X cells in articular cartilage. All data are shown as means±standard deviations. n=8/group. *P<0.05, **P<0.01. NS, no significant difference;

FIG. 3-2A, FIG. 3-2B, FIG. 3-2C, FIG. 3-2D, FIG. 3-2E, FIG. 3-2F, FIG. 3-2G, FIG. 3-2H, FIG. 3-21. and FIG. 3-2J demonstrate that sensory nerve innervation in subchondral bone decreased with PTH treatment. (FIG. 3-2A) Immunofluorescence analysis of CGRP⁺ (1 row, green), Substance P⁺ (2^{nd} row, red), P2X3′(3^{th} row, red), NF200⁺ (4^{th} row, red), PIZEO⁺ (5^{th} row, red) sensory nerve fibers and PGP9.5⁺ (6^{th} row, red) nerve fibers in tibial subchondral bone after DMM in sham-operated, PTH-treated DMM and vehicle-treated DMM mice. DAPI stains nuclei (blue). Scale Bar, 50 μm. (FIG. 3-2B-FIG. 3-2G) The quantitative analysis of the density of CGRP⁺, SP⁺, P2X3⁺, NF200⁺, PIZEO⁺ sensory nerve fibers and PGP9.5⁺ nerve fibers in tibial subchondral bone after DMM. n=8/group. (FIG. 3-2H) Immunofluorescence analysis of CGRP⁺ (top, green) sensory nerve fibers and PGP9.5⁺ (bottom, red) nerve fibers in the synoviμm after DMM in sham-operated. PTH-treated and vehicle-treated DMM mice. DAPI stains nuclei (blue). Scale Bar, 50 μm. (FIG. 3-2I, FIG. 3-2J) The quantitative analysis of the density of CGRP⁺, sensory nerve fibers and PGP9.5+ nerve fibers in the synoviμm after DMM in sham-operated, PTH-treated DMM and vehicle-treated DMM mice. n=8/group. *P<0.05, **P<0.01. NS, no significant difference:

FIG. 3-3A, FIG. 3-3B, FIG. 3-3C, FIG. 3-3D, FIG. 3-3E, FIG. 3-3F, and FIG. 3-3G demonstrate that PTH sustains subchondral bone microarchitecture by remodeling. (FIG. 3-3A) Top row: Three-dimensional high-resolution ICT images of tibial subchondral bone medial compartment (sagittal view) at 8 weeks post sham-operated, PTH-treated DMM and vehicle-treated DMM. Scale bar: 1 mm. Bottom row: Immunohistochemical analysis of COX2⁺ cells in mouse tibial subchondral bone after DMM surgery. Scale bar: 50 μm. (FIG. 3-3B-FIG. 3-3E) Quantitative analysis of structural parameters of subchondral bone by CT analysis: thickness of subchondral bone plates (SBP.Th), trabecular pattern factor (Tb. Pf), structure model index (SMI) and total volume of pore space Po.V (tot). n=8/group. (FIG. 3-3F) The quantitative analysis of COX2⁺ cells in mouse tibial subchondral bone. n=8/group. (FIG. 3-3G) Quantitative analysis of PGE₂ in subchondral bone determined by Elisa. n=8/group. (H) Trichrome staining in tibial subchondral bone sections. Scale bar: 50 μm. (I) Calcein (green) and alizarin red (red) fluorescent double labeling. Scale bar: 50 μm. *P<0.05, **P<0.01;

FIG. 3-4A, FIG. 3-4B, FIG. 3-4C, FIG. 3-4D, FIG. 3-4E, FIG. 3-4F, FIG. 3-4G, FIG. 3-4H, FIG. 3-4I, and FIG. 3-4J demonstrate that PTH sustains subchondral bone remodeling by endocytosis of TGFβIIR. (FIG. 3-4A) Immunofluorescence or immunohistochemical analysis and quantification of nestin⁺ cells (top, green) and osterix⁺ cells (bottom, brown) in tibial subchondral bone after sham operation PTH-treated DMM, or vehicle-treated DMM mice. Scale bar: 50 μm. (FIG. 3-4B, FIG. 3-4C) The quantification of nestin⁺ cells and osterix⁺ cells in tibial subchondral bone in different groups. n=8/group. (FIG. 3-4D, FIG. 3-4F) The immunohistochemical analysis and quantification of pSmad2/3⁺ cells (brown) in mouse tibial subchondral bone of sham-operated. PTH-treated or Vehicle-treated DMM mice. Scale bar, 50 μm; n=8/group. (FIG. 3-4E, FIG. 3-4G) The TRAP staining (pink) and quantitative analysis of TRAP⁺ cells in mouse tibial subchondral bone of sham-operated, PTH-treated or Vehicle-treated DMM mice. Scale bar: 100 μm. n=8/group. (FIG. 3-4H) Quantitative analysis of active TGFβ in serum determined by Elisa. n=8/group. (FIG. 3-4I) Immunofluorescent analysis of TGFβIIR (green) distribution on mouse BMSC. Actin (red); DAPI stains nuclei (blue) Scale bar, 20 μm. (FIG. 3-4J) Immunofluorescent analysis of pSmad2/3⁺ on mouse BMSC. Scale bar, 50 μm. DAPI stains nuclei (blue). *P<0.05, **P<0.01;

FIG. 3-5A, FIG. 3-5B, FIG. 3-5C, FIG. 3-5D, FIG. 3-5E, FIG. 3-5F, FIG. 3-50, FIG. 3-5H, FIG. 3-51, FIG. 3-5J, and FIG. 3-5K demonstrate that delayed PTH attenuates progressive OA pain and joint degeneration in DMM model. (FIG. 3-5A, FIG. 3-5B) PWT at the left hind paw and withdrawal threshold tested by PAM at left knee joint in sham-operated, PTH-treated DMM and vehicle-treated DMM mice, starting from 4 weeks to 8 weeks after surgery. n=8/group. (FIG. 3-5C) Quantitative analysis of percentage of LH paw intensity, LH area and LH swing speed relative to RH, based on CatWalk analysis. n=8/group. (FIG. 3-5D) Immunofluorescent analysis of the density of CGRP⁺ (top, green) and SP⁺ (bottom, red) sensory nerve fibers in tibial subchondral bone of sham-operated, PTH-treated DMM and vehicle-treated DMM mice. Scale bar, 50 μm. (FIG. 3-5E, FIG. 3-5F) The quantitative analysis of the density of CGRP⁺, SP⁺ sensory nerve fibers in tibial subchondral bone after DMM in sham-operated, PTH-treated DMM and vehicle-treated DMM mice. DAPI stains nuclei (blue). n=8/group. (FIG. 3-5G) Safranin O-Fast green staining of sagittal sections of tibia medial compartment, proteoglycan (red) and bone (green) and OARSI. Scale bar, 500 μm. n=8/group. (FIG. 3-5H) Three-dimensional high-resolution PCT images of tibial subchondral bone medial compartment (sagittal view) post sham-operated, PTH-treated DMM and vehicle-treated DMM (top). Scale bar: 1 mm. Immunohistochemical analysis of COX2⁺ cells in mouse tibial subchondral bone (bottom, brown). Scale bar, 50 μm. (FIG. 3-5I) Quantitative analysis of structural parameters of subchondral bone by μCT analysis: SBP.Th, Th. Pf, SMI and Po.V(tot). n=8/group. (FIG. 3-5J) Immunohistochemical analysis and quantitative analysis of COX2⁺ cells in mouse tibial subchondral bone. Quantitative analysis of PGE₂ in subchondral bone determined by Elisa. n=8/group. (FIG. 3-5K) Immunohistochemical or immunofluorescent analysis and quantitative analysis of osterix+(brown) cells in tibial subchondral bone. Scale bar, 50 μm. n=8/group. *P<0.05, **P<0.01:

FIG. 3-6A, FIG. 3-6B, FIG. 3-6C, FIG. 3-6D, FIG. 3-6E, FIG. 3-6F, and FIG. 3-6G demonstrate that PTH-induced OA pain relief inhibited by PTH1R knockout on Nestin⁺ MSCs. (FIG. 3-6A, FIG. 3-6B) PWT at the left hind paw and withdrawal threshold tested by PAM at left knee joint in sham-operated, PTH-treated DMM and vehicle-treated DMM PTH1^{-/-} and PTH1R^{+/+} mice. n=8/group. (FIG. 3-6C) Quantitative analysis of LH paw intensity. LH area and LH swing speed relative to RH in sham-operated, PTH-treated DMM and vehicle-treated DMM PTH1 R^{-/-} and PTH1R^{+/+} mice, based on CatWalk analysis. n=8/group. (FIG. 3-6D) Immunofluorescent analysis of CGRP⁺ (1^{st} row, green), Substance P⁺ (2^{nd} row, red), P2X3⁺ (3^{rd} row, red), NF200⁺ (4^{th} row, red), PIZEO⁺ (5^{th} row, red) sensory nerve fibers and PGP9.5⁺ (6^{th} row, red) nerve fibers in tibial subchondral bone of PTH-treated or vehicle-treated PTH1R^{-/-} and PTH1R^{+/+} mice. Scale bar, 50 μm. (FIG. 3-6E) The quantitative analysis of the density of CGRP⁺, SP⁺, P2X3⁺, NF200⁺, PIZEO⁺ sensory nerve fibers and PGP9.5⁺ nerve fibers in tibial subchondral bone of sham-operated, PTH-treated DMM and vehicle-treated DMM PTH1R⁺ and PTH1R^{+/+} mice. n=8/group. (FIG. 3-6F, FIG. 3-6G) Immunofluorescent and quantitative analysis of CGRP⁺ (top, green) sensory nerve fibers and PGP9.5⁺ (bottom, red) nerve fibers of sham-operated, PTH-treated DMM and vehicle-treated DMM PTH1R^{-/-} and PTH1R^{+/+} mice. Scale bar, 50 μm. n=8/group. *P<0.05, **P<0.01. NS, no significant difference, and

FIG. 3-7A. FIG. 3-713. FIG. 3-7C, FIG. 3-7D, FIG. 3-7E, FIG. 3-7F, and FIG. 3-7G. and FIG. 3-7H demonstrate that PTH-induced bone remodeling inhibited by PTH1R knockout on Nestin⁺ MSCs. (FIG. 3-7A) Top: Safranin O-Fast green staining of sagittal sections of tibia medial compartment, proteoglycan (red) and bone (green). Scale bar, 500 μm; Middle. three-dimensional high-resolution μCT images of tibial subchondral bone medial compartment. Scale bar 1 mm: Bottom: Immunohistochemical analysis of COX2⁺ (brown) cells in mouse tibial subchondral bone. Scale bar, 50 μm: (FIG. 3-7B) OARSI score. N=8/group (FIG. 3-7C) Quantitative analysis of structural parameters of subchondral bone by μCT analysis: SBP.Th, Th. Pf, SMI and Po.V(tot). n=8/group. (FIG. 3-7D) The quantitative analysis of COX2⁺ cells in mouse tibial subchondral bone. n=8/group. (FIG. 3-7E) Quantitative analysis of PGE2 in subchondral bone determined by Elisa. n=8/group. (FIG. 3-7F) Immunohistochemical analysis and quantification of pSmad2/3⁺ cell in subchondral bone marrow. Scale bar, 50 μm; n=8/group. (FIG. 3-7G, FIG. 3-7H) The Immunofluorescent or immunohistochemical analysis and quantification of nestin+ cells (green) and osterix⁺ (brown) cells in tibial subchondral bone. Scale bar, 50 μm; n=8/group. *P<0.05, **P<0.01. NS, no significant difference.

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein: rather, these embodiments are provided so that this disclosure will satisfy applicable legal requirements. Indeed. many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

### I. Parathyroid Hormone Attenuates Low Back Pain and Osteoarthritic Pain

In some embodiments, the presently disclosed subject matter provides a method for treating low back pain (LBP) and/or osteoarthritic pain in a subject in need of treatment thereof, the method comprising administering to the subject a composition comprising a recombinant parathyroid hormone (PTH) and a pharmaceutically acceptable carrier.

In certain embodiments, the low back pain comprises a nonspecific low back pain.

In certain embodiments, the administering of the recombinant parathyroid hormone (PTH) inhibits osteoclast activity-induced sensory innervation in a vertebral endplate of the subject.

As used herein, the term “inhibit,” and grammatical derivations thereof, refers to the ability of a presently disclosed compound, e.g., a recombinant parathyroid hormone (PTH), to block, partially block, interfere, decrease. or reduce, for example, osteoclast activity-induced sensory innervation in a vertebral endplate. Thus, one of ordinary skill in the art would appreciate that the term “inhibit” encompasses a complete and/or partial decrease in an activity, e.g., a decrease by at least 10%. in some embodiments, a decrease by at least 20%, 30%, 50%, 75%, 95%, 98%, and up to and including 100%.

In particular embodiments, the administering of the recombinant parathyroid hormone (PTH) treats the osteoarthritic pain by one or more of inhibition of nerve innervation, inhibition of subchondral bone deterioration, inhibition of articular cartilage degeneration. attenuation of joint degeneration, decelerating subchondral bone deterioration, and sustaining of subchondral bone microarchitecture by remodeling.

In other embodiments, the administering of the recombinant parathyroid hormone (PTH) increases the intervertebral disc (IV D) space by decreasing the volume and porosity of sclerotic endplates.

In yet other embodiments, the administering of the recombinant parathyroid hormone (PTH) prevents endplate remodeling and sclerosis.

In even yet other embodiments, the administering of the recombinant parathyroid hormone (PTH) reduces sensory nerve fibers.

In other embodiments, the administering of the recombinant parathyroid hormone (PTH) reduces the porosity of sclerotic endplates.

In some embodiments, the method further comprises administering at least one other agent in combination with the administering of the recombinant parathyroid hormone (PTH).

In certain embodiments, the at least one other agent is selected from the group consisting of paracetamol, an opioid, a non-steroidal anti-inflammatory drug, a skeletal muscle relaxant, a triptan, an α2-agonist, a local anesthetic, a tricyclic antidepressant, a benzodiazepine, a steroid, a visco supplement, and combinations thereof.

In particular embodiments, the low back pain is associated with one or more of spine degeneration, lumbar disc herniation (LDH), scoliosis, cancer, and an infection.

In some embodiments, the recombinant PTH comprises a full-length PTH protein or a fragment of PTH. In particular embodiments, the recombinant parathyroid hormone comprises teriparatide (PTH(I-34′)). In other embodiments, the recombinant parathyroid hormone comprises an intact parathyroid hormone (iPTH).

In certain embodiments, the composition is administered to the subject at least once a day.

In other embodiments, the presently disclosed subject matter provides the use of a recombinant parathyroid hormone to treat low back pain (LBP) or osteoarthritic pain in a subject in need thereof.

As used herein, the term “treating” can include reversing, alleviating, inhibiting the progression of, preventing or reducing the likelihood of the disease, disorder, or condition to which such term applies, or one or more symptoms or manifestations of such disease, disorder or condition. Preventing refers to causing a disease, disorder, condition, or symptom or manifestation of such, or worsening of the severity of such, not to occur. Accordingly, the presently disclosed compounds can be administered prophylactically to prevent or reduce the incidence or recurrence of the disease, disorder, or condition.

The “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes. such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like: equines, e.g., horses. donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

In general, the “effective amount” of an active agent or drug delivery device refers to the amount necessary to elicit the desired biological response. As will be appreciated by those of ordinary skill in this art, the effective amount of an agent or device may vary depending on such factors as the desired biological endpoint, the agent to be delivered, the makeup of the pharmaceutical composition, the target tissue, and the like.

The term “combination” is used in its broadest sense and means that a subject is administered at least two agents, more particularly a recombinant parathyroid hormone (PTH) and at least one other agent. In some embodiments, the at least one other agent is selected from the group consisting of paracetamol, opioids, non-steroidal anti-inflammatory drugs (NSAIDs, including COX-2 inhibitors), and skeletal muscle relaxants. In other embodiments, the at least one other agent is selected from the group consisting of triptans, α2-agonists, and local anesthetics. In chronic cases, the at least one other agents is a tricyclic antidepressant and/or a benzodiazepine.

More particularly, the term “in combination” refers to the concomitant administration of two (or more) active agents for the treatment of a, e.g., single disease state. As used herein, the active agents may be combined and administered in a single dosage form, may be administered as separate dosage forms at the same time, or may be administered as separate dosage forms that are administered alternately or sequentially on the same or separate days. In one embodiment of the presently disclosed subject matter. the active agents are combined and administered in a single dosage form. In another embodiment, the active agents are administered in separate dosage forms (e.g., wherein it is desirable to vary the amount of one but not the other). The single dosage form may include additional active agents for the treatment of the disease state.

Further, the recombinant parathyroid hormone (PTH) described herein can be administered alone or in combination with adjuvants that enhance stability of the recombinant parathyroid hormone (PTH), alone or in combination with one or more antibacterial agents, facilitate administration of pharmaceutical compositions containing them in certain embodiments, provide increased dissolution or dispersion, increase inhibitory activity, provide adjunct therapy, and the like, including other active ingredients. Advantageously, such combination therapies utilize lower dosages of the conventional therapeutics, thus avoiding possible toxicity and adverse side effects incurred when those agents are used as monotherapies.

The timing of administration of a recombinant parathyroid hormone (PTH) and at least one additional therapeutic agent can be varied so long as the beneficial effects of the combination of these agents are achieved. Accordingly, the phrase “in combination with” refers to the administration of a recombinant parathyroid hormone (PTH) and at least one additional therapeutic agent either simultaneously, sequentially, or a combination thereof. Therefore, a subject administered a combination of a recombinant parathyroid hormone (PTH) and at least one additional therapeutic agent can receive a recombinant parathyroid hormone (PTH) and at least one additional therapeutic agent at the same time (i.e., simultaneously) or at different times (i.e., sequentially, in either order, on the same day or on different days), so long as the effect of the combination of both agents is achieved in the subject.

When administered sequentially, the agents can be administered within 1, 5, 10, 30, 60, 120, 180, 240 minutes or longer of one another. In other embodiments, agents administered sequentially, can be administered within 1, 5, 10, 15, 20 or more days of one another. Where the recombinant parathyroid hormone (PTH) and at least one additional therapeutic agent are administered simultaneously, they can be administered to the subject as separate pharmaceutical compositions, each comprising either a recombinant parathyroid hormone (PTH) or at least one additional therapeutic agent, or they can be administered to a subject as a single pharmaceutical composition comprising both agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times.

In some embodiments, when administered in combination, the two or more agents can have a synergistic effect. As used herein, the terms “synergy,” “synergistic,” “synergistically” and derivations thereof, such as in a “synergistic effect” or a “synergistic combination” or a “synergistic composition” refer to circumstances under which the biological activity of a combination of a recombinant parathyroid hormone (PTH) and at least one additional therapeutic agent is greater than the sum of the biological activities of the respective agents when administered individually.

Synergy can be expressed in terms of a “Synergy Index (SI),” which generally can be determined by the method described by F. C. Kull et al., Applied Microbiology 9, 538 (1961), from the ratio determined by:

Qₐ/Q_{A}+Q_{b}/Q_{B}=Synergy Index (SI)

wherein:

Q_{A} is the concentration of a component A. acting alone, which produced an end point in relation to component A;
Qₐ is the concentration of component A, in a mixture, which produced an end point;
Q_{B} is the concentration of a component B. acting alone, which produced an end point in relation to component B: and
Q_{b} is the concentration of component B, in a mixture, which produced an end point.

Generally. when the sum of Qₐ/Q_{A} and Q_{b}/Q_{B} is greater than one, antagonism is indicated. When the sum is equal to one, additivity is indicated. When the sum is less than one. synergism is demonstrated. The lower the SI, the greater the synergy shown by that particular mixture. Thus, a “synergistic combination” has an activity higher that what can be expected based on the observed activities of the individual components when used alone. Further, a “synergistically effective amount” of a component refers to the amount of the component necessary to elicit a synergistic effect in, for example, another therapeutic agent present in the composition.

### Pharmaceutical Compositions and Administration

In another aspect, the present disclosure provides a pharmaceutical composition including a recombinant parathyroid hormone (PTH) alone or in combination with one or more additional therapeutic agents in admixture with a pharmaceutically acceptable excipient. One of skill in the art will recognize that the pharmaceutical compositions include the pharmaceutically acceptable salts of the compounds described above.

In therapeutic and/or diagnostic applications, the compounds of the disclosure can be formulated for a variety of modes of administration, including systemic and topical or localized administration. Techniques and formulations generally may be found in Remington: The Science and Practice of Pharmacy (20^{th} ed.) Lippincott, Williams & Wilkins (2000).

Depending on the specific conditions being treated, such agents may be formulated into liquid or solid dosage forms and administered systemically or locally. The agents may be delivered, for example, in a timed- or sustained-slow release form as is known to those skilled in the art. Techniques for formulation and administration may be found in Remington: The Science and Practice of Pharmacy (20^{th} ed.) Lippincott, Williams & Wilkins (2000). Suitable routes may include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articular, intra-sternal, intra-synovial, intra-hepatic, intralesional, intracranial, intraperitoneal, intranasal, or intraocular injections or other modes of delivery.

For injection, the agents of the disclosure may be formulated and diluted in aqueous solutions. such as in physiologically compatible buffers such as Hank's solution, Ringer's solution, or physiological saline buffer. For such transmucosal administration, penetrants appropriate to the barrier to be permeated are used in the formulation. Such penetrants are generally known in the art.

Use of pharmaceutically acceptable inert carriers to formulate the compounds herein disclosed for the practice of the disclosure into dosages suitable for systemic administration is within the scope of the disclosure. With proper choice of carrier and suitable manufacturing practice, the compositions of the present disclosure, in particular. those formulated as solutions. may be administered parenterally, such as by intravenous injection. The compounds can be formulated readily using pharmaceutically acceptable carriers well known in the art into dosages suitable for oral administration. Such carriers enable the compounds of the disclosure to be formulated as tablets, pills, capsules, liquids, gels, syrups, slurries, suspensions and the like, for oral ingestion by a subject (e.g., patient) to be treated.

For nasal or inhalation delivery, the agents of the disclosure also may be formulated by methods known to those of skill in the art, and may include, for example, but not limited to, examples of solubilizing. diluting, or dispersing substances. such as saline; preservatives, such as benzyl alcohol: absorption promoters; and fluorocarbons.

Pharmaceutical compositions suitable for use in the present disclosure include compositions wherein the active ingredients are contained in an effective amount to achieve its intended purpose. Determination of the effective amounts is well within the capability of those skilled in the art, especially in light of the detailed disclosure provided herein. Generally, the compounds according to the disclosure are effective over a wide dosage range. For example, in the treatment of adult humans, dosages from 0.01 to 1000 mg, from 0.5 to 100 mg. from 1 to 50 mg per day. and from 5 to 40 mg per day are examples of dosages that may be used. A non-limiting dosage is 10 to 30 mg per day. The exact dosage w % ill depend upon the route of administration, the form in which the compound is administered, the subject to be treated, the body weight of the subject to be treated, the bioavailability of the compound(s), the adsorption, distribution, metabolism, and excretion (ADME) toxicity of the compound(s), and the preference and experience of the attending physician.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions.

Pharmaceutical preparations for oral use can be obtained by combining the active compounds with solid excipients, optionally grinding a resulting mixture, and processing the mixture of granules. after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). If desired. disintegrating agents may be added, such as the cross-linked polyvinylpyrrolidone, agar, or alginic acid or a salt thereof such as sodium alginate.

Dragee cores are provided with suitable coatings. For this purpose, concentrated sugar solutions may be used. which may optionally contain gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols (PEGs). In addition, stabilizers may be added.

### Definitions

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, +100% in some embodiments±50%, in some embodiments±20%, in some embodiments±10%, in some embodiments f 5%, in some embodiments ±1%, in some embodiments±0.5%, and in some embodiments t 0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### Sensory Innervation in Porous Endplates by Netrin-1 from Osteoclasts Mediates PGE2-induced Spinal Hypersensitivity in Mice

### 1.1 Overview

Spinal pain is a major clinical problem. Its origins and underlying mechanisms, however, remain unclear. In some embodiments, the presently disclosed subject matter provides that in mice, osteoclasts induce sensory innervation in the porous endplates, which contributes spinal hypersensitivity in mice. Sensory innervation of the porous areas of sclerotic endplates in mice was confirmed. Lumbar spine instability (LSI), or aging, induces spinal hypersensitivity in mice. In these conditions, elevated levels of PGE2, which activate sensory nerves leading to sodium influx through Naᵥ1.8 channels, were observed. Further, knockout of PGE2 receptor 4 in sensory nerves significantly reduced spinal hypersensitivity. Inhibition of osteoclast formation by knockout Rankl in the osteocytes significantly inhibited LSI-induced porosity of endplates, sensory innervation, and spinal hypersensitivity. Knockout of Netrin-1 in osteoclasts abrogates sensory innervation into porous endplates and spinal hypersensitivity. These findings suggest that osteoclast-initiated porosity of endplates and sensory innervation are potential therapeutic targets for spinal pain.

### 1.2 Background

Low back pain (LBP) is a common health problem, which most people (80%) experience at some point, especially in older adults. Rubin, 2007: Hartvigsen et al., 2006; Hartvigsen et al., 2004: and Global, regional, and national incidence, prevalence, and years lived with disability for 310 diseases and injuries, 1990-2015: a systematic analysis for the Global Burden of Disease Study 2015.

In the United States alone, the direct and indirect costs associated with LBP surpass $90 billion per year, with similar adjusted rates in other countries. Samartzis and Grivas, 2017. 90% of LBP is nonspecific LBP, which has no apparent pathoanatomical cause. Krismer and van Tulder, 2007: Koes et al., 2006. Several lumbar structures, such as intervertebral disc, facet joints, are plausible sources of nonspecific LBP, but the pain cannot be reliably attributed to those structures by clinical tests. Hancock et al., 2007; Maher et al., 2017; and Hartvigsen et al., 2018. Importantly, intervertebral disc (IVD) degeneration is frequently observed in asymptomatic patients, indicating that disc degeneration, per se, is not painful in some patients. Hurn and Karppinen, 2004: Borenstein et al., 2001. Hence, identifying the source of LBP and related mechanisms is essential to develop effective treatments for LBP.

The positive association between vertebral endplate signal changes (i.e., Modic changes) and LBP has been shown by magnetic resonance imaging (MRI) examination. Rahme and Moussa, 2008; Luoma et al., 2016. Modic changes are common MRI findings in patients with nonspecific LBP. It is believed to be a factor independently associated with the increased risk of LBP. Jensen et al., 2008: Maatta et al., 2015: and Jensen et al., 2014. The size of Modic change lesions positively correlates with LBP. Jarvinen et al., 2015. Histological analysis further showed that the presence of endplate lesions was associated with LBP. Wang et al., 2012. During aging, endplates undergo ossification with elevated osteoclasts and become porous. Bian et al., 2016: Bian et al., 2017; Rodriguez et al., 2012; and Papadakis et al., 2011. Histological and micro CT analysis further revealed that sclerotic endplates are highly porous. Rodriguez et al., 2012. Progressively more porous endplates with narrowed IVD space are characteristics of spinal degeneration. Rodriguez et al., 2012; Taher et al., 2012. Since pain is produced by nociceptors, LBP may be caused by sensory innervation into endplates. Moreover, nerve density was higher in porous endplates than in normal endplates or degenerative nucleus pulposus. Fields et al., 2014. Zoledronic acid and denosumab, drugs that inhibit osteoclast activities, have shown analgesic effects in patients with Modic changes associated LBP, Cai et al., 2018; Koivisto et al., 2014, with the implication of a potential role of osteoclast activity in sensory nerve innervation.

Prostaglandin E2 (PGE2) is an inflammatory mediator released at the focal inflamed tissue and a neuromodulator that alters neuronal excitability. Four types of G-protein-coupled EP receptors (EP1-EP4) mediate the functions of PGE2. EP4 receptor is considered the primary mediator of PGE2-evoked inflammatory pain hypersensitivity and sensitization of sensory neurons. Lin et al., 2006: Southall and Vasko, 2001. It was recently reported that PGE2, produced from arachidonic acid by the enzymatic activity of cyclooxygenase 2 (COX2), during bone remodeling activates PGE2 receptor 4 (EP4) in CGRP⁺ sensory nerves to tune down sympathetic tones further inducing osteoblastic differentiation of MSCs. Chen et al, 2019. Specific EP4 receptor antagonists could reduce acute and chronic pain, including osteoarthritis pain. Lin et al., 2006; Clark et al., 2008: Kirkby Shaw et al., 2016; Nakao et al., 2007. The tetrodotoxin-resistant (TX-R) sodium channel Naᵥ1.8 is a potential drug target for pain. Naᵥ1.8 is expressed primarily in small and medium-sized dorsal root ganglion (DRG) neurons and their fibers. Janis et al., 2007: Coggeshall et al., 2004: and Schuelert and McDougall, 2012. PGE2 could modulate the TTX-R sodium current in DRG neurons and promote Naᵥ1.8 trafficking to the cell surface. Liu et al., 2010; England et al., 1996.

One aspect of the presently disclosed study is to demonstrate that osteoclasts initiate porosity of endplates with sensory innervation into porous areas. The presently disclosed data show that Calcitonin Gene-Related Peptide positive (CGRP⁺) nociceptive nerve fibers and blood vessels were increased in the cavities of sclerotic endplates. The elevated PGE2 in porous endplates induces sodium influx into the cells to stimulate sensory nerves that leads to spinal pain. Inhibition of osteoclast activity attenuated sensory innervation in porous endplates and pain behavior.

### 1.3 Results

### 1.3.1 Development of Hyperalgesia in LSI and Aged Mouse Models

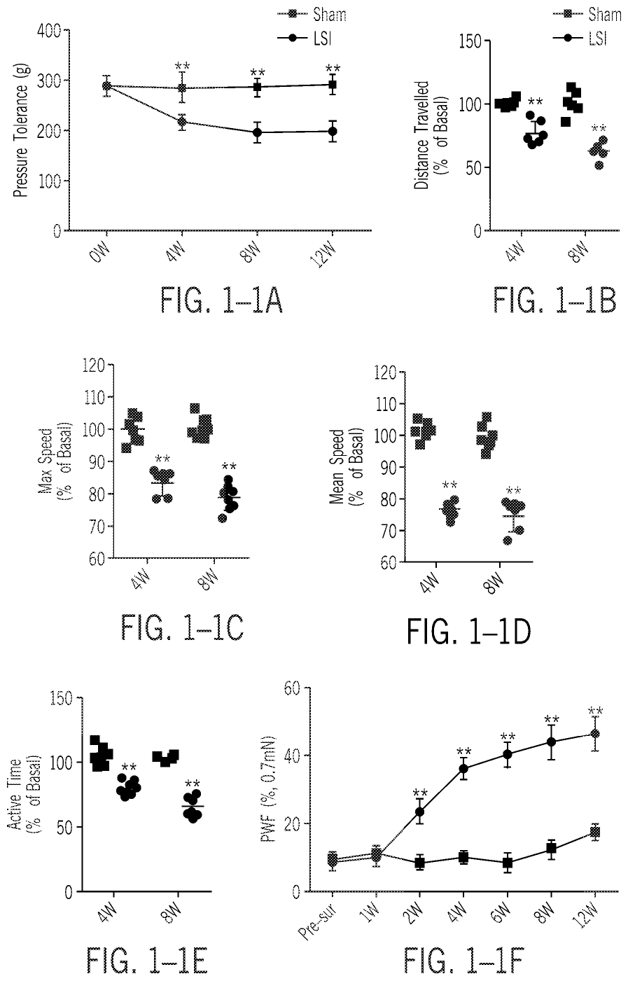

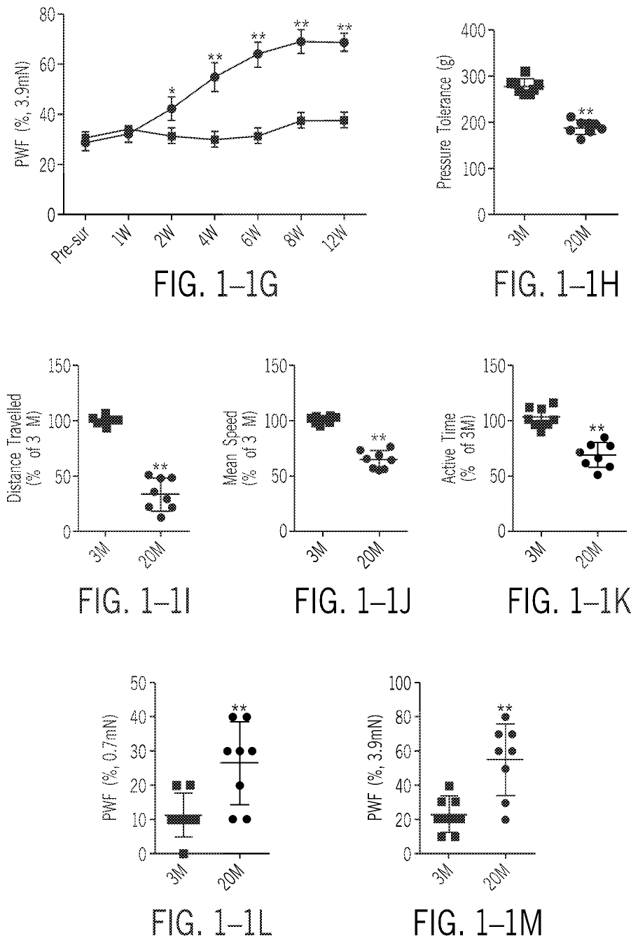

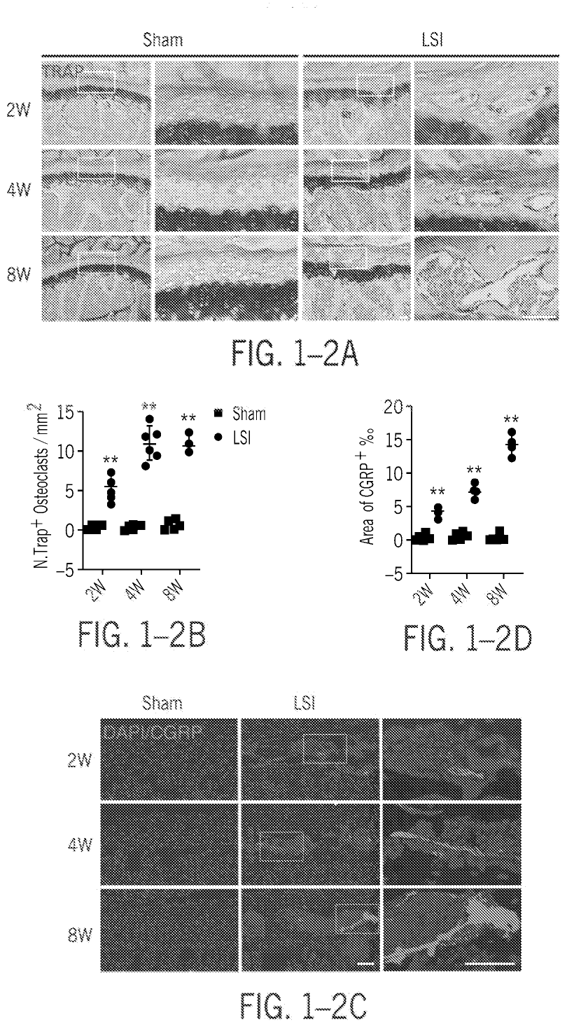

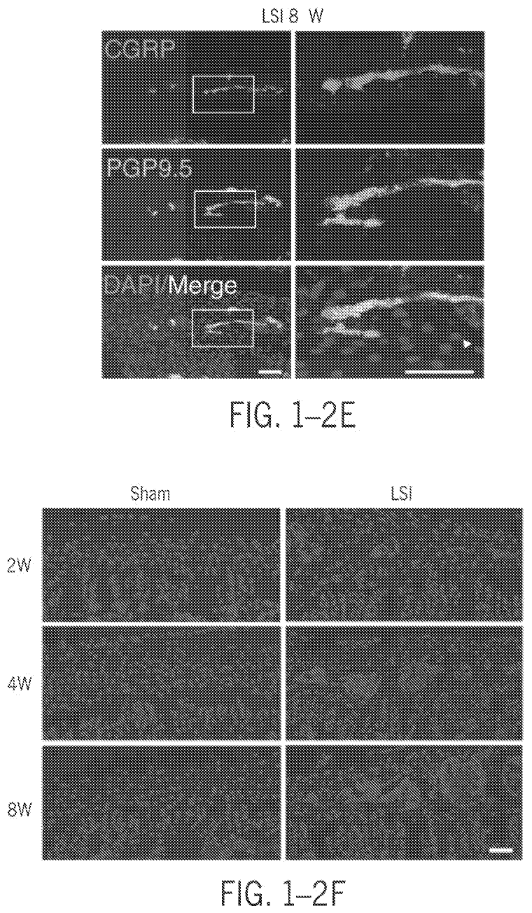

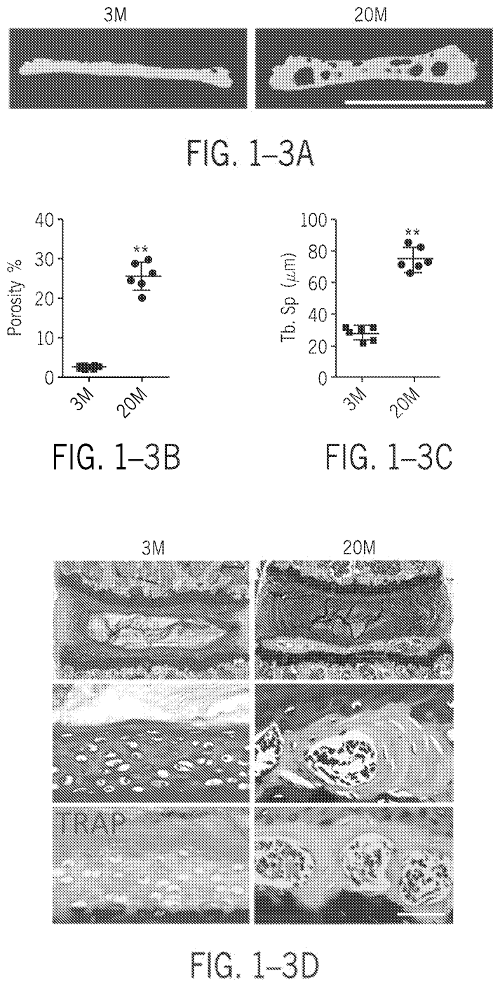

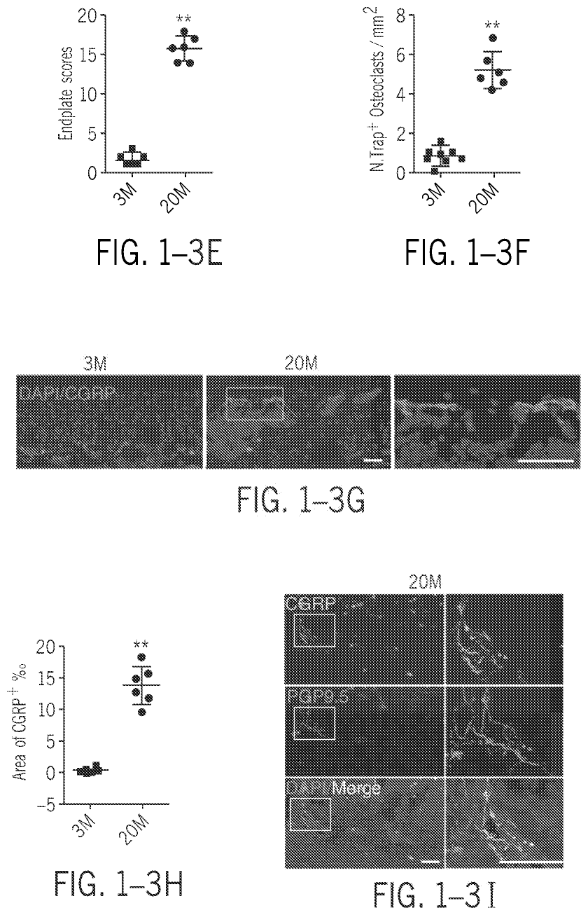

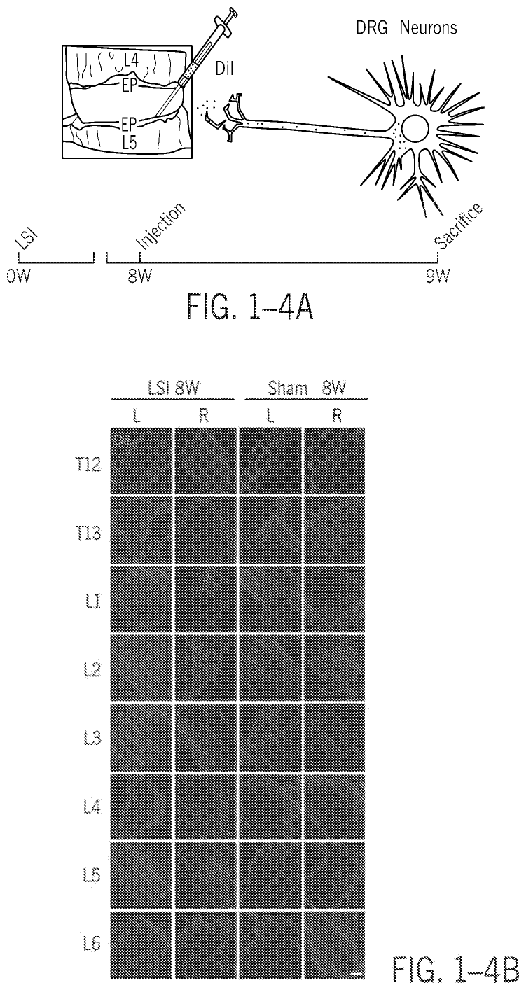

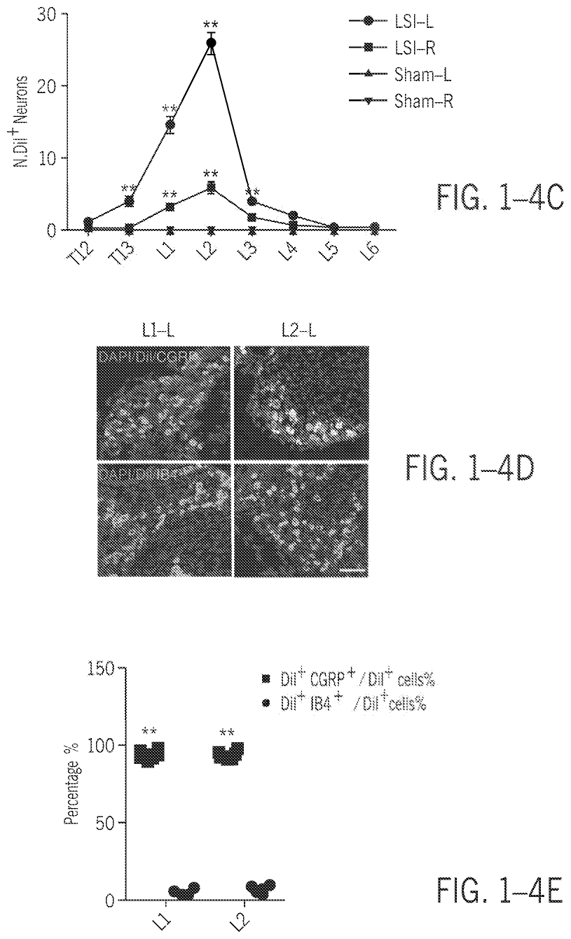

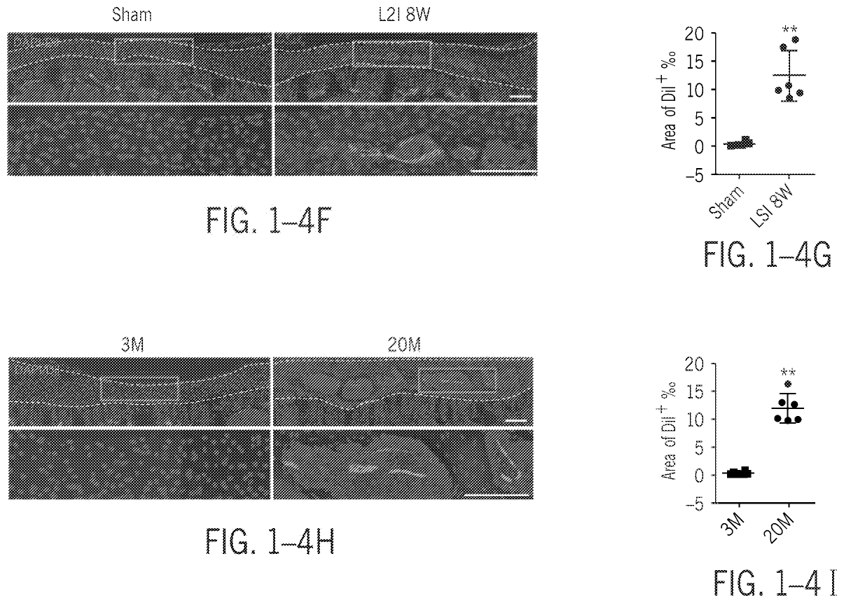

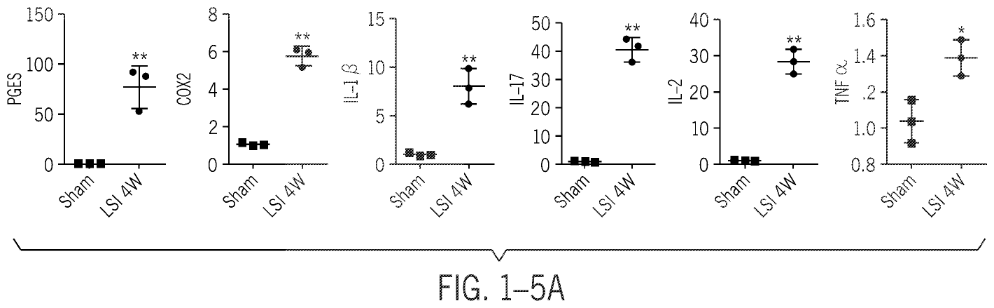

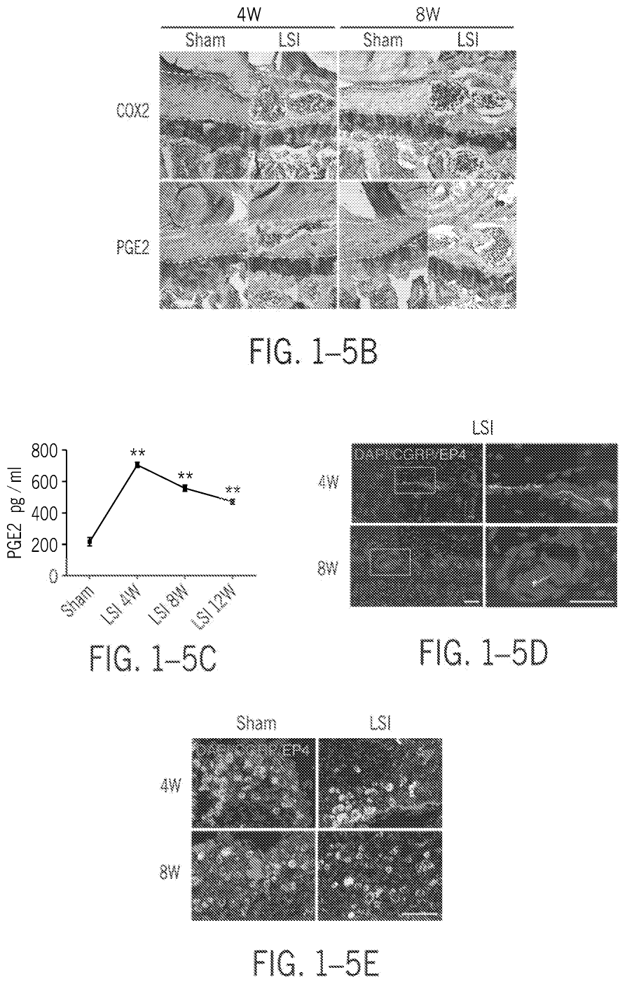

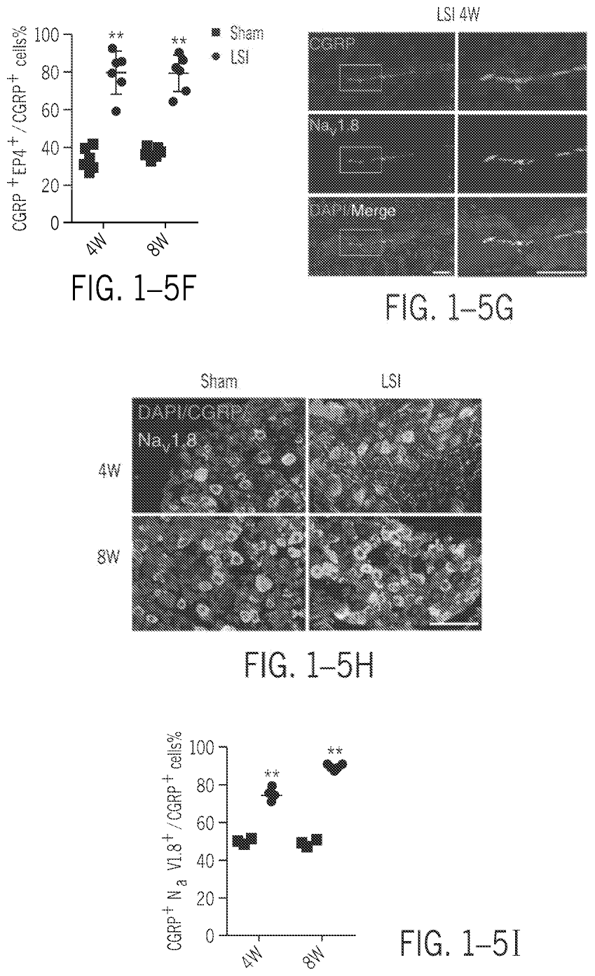

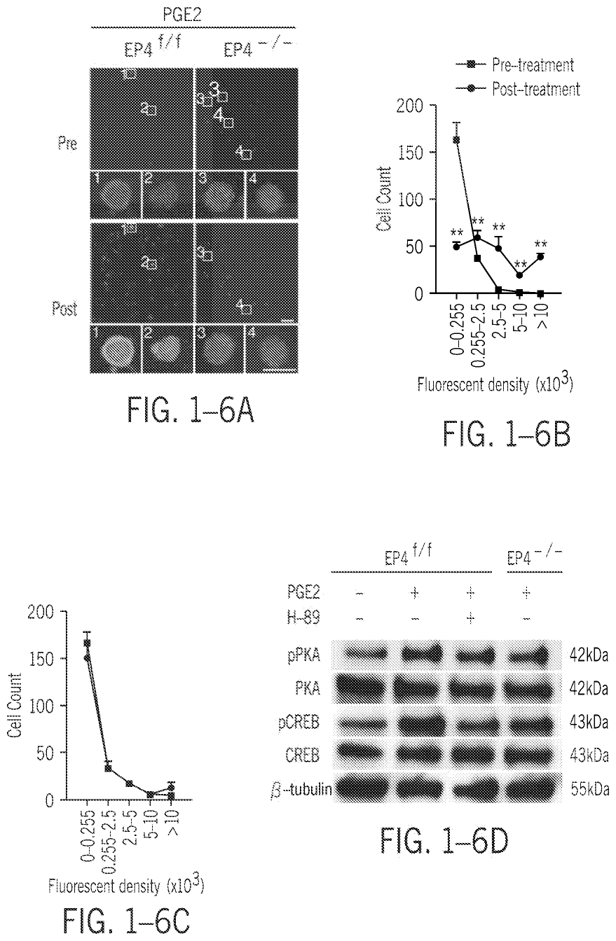

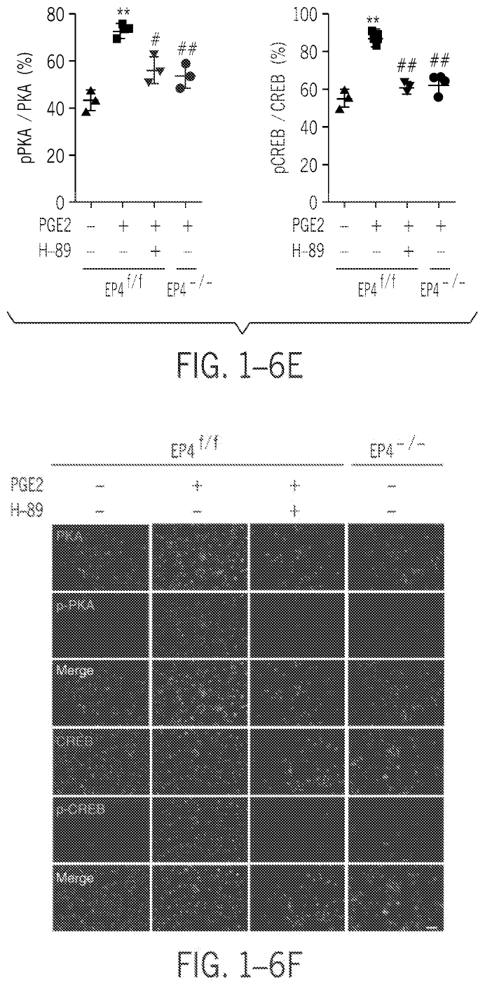

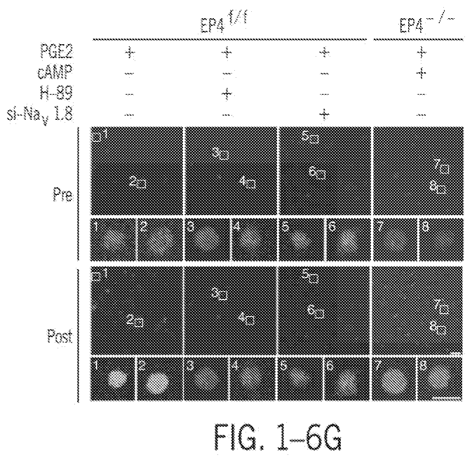

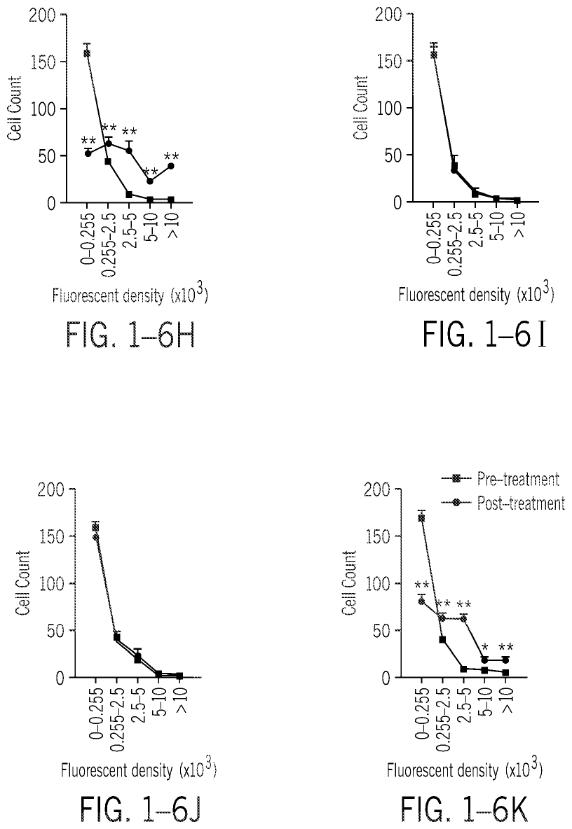

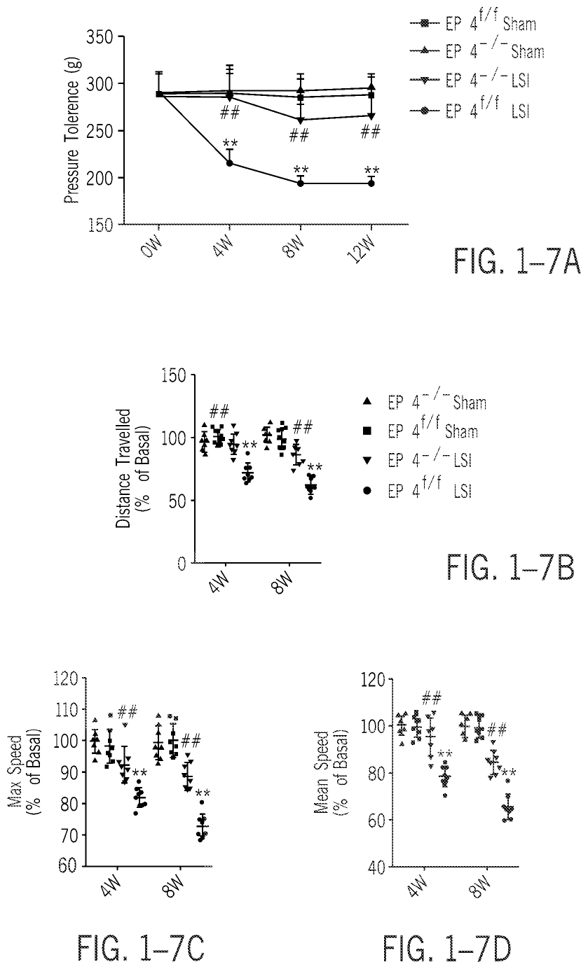

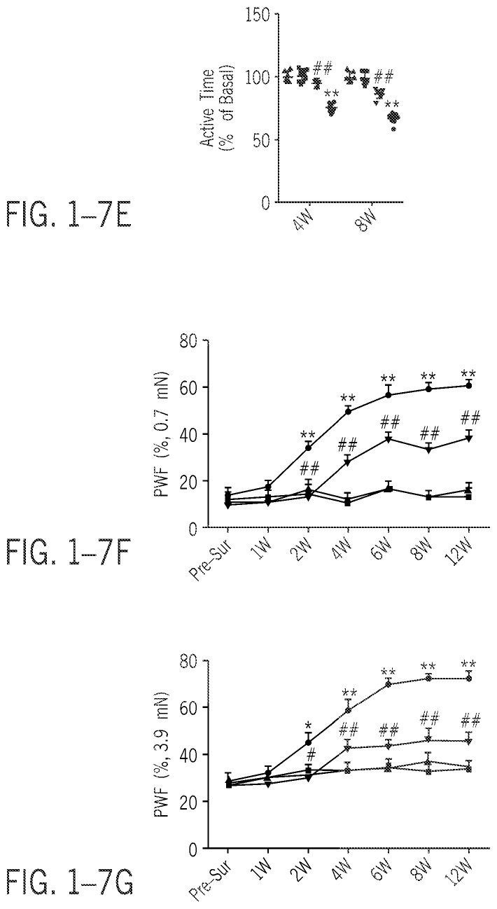

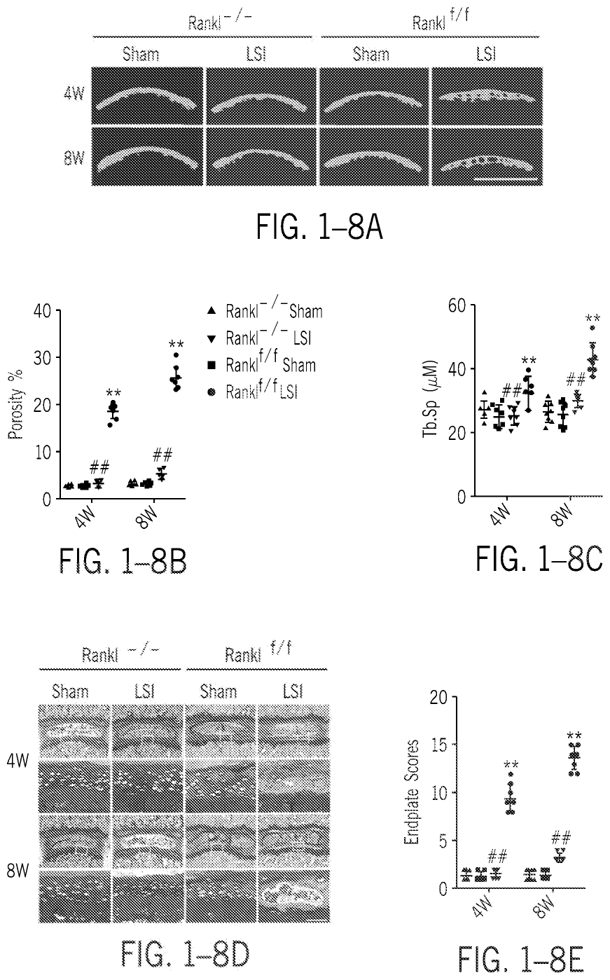

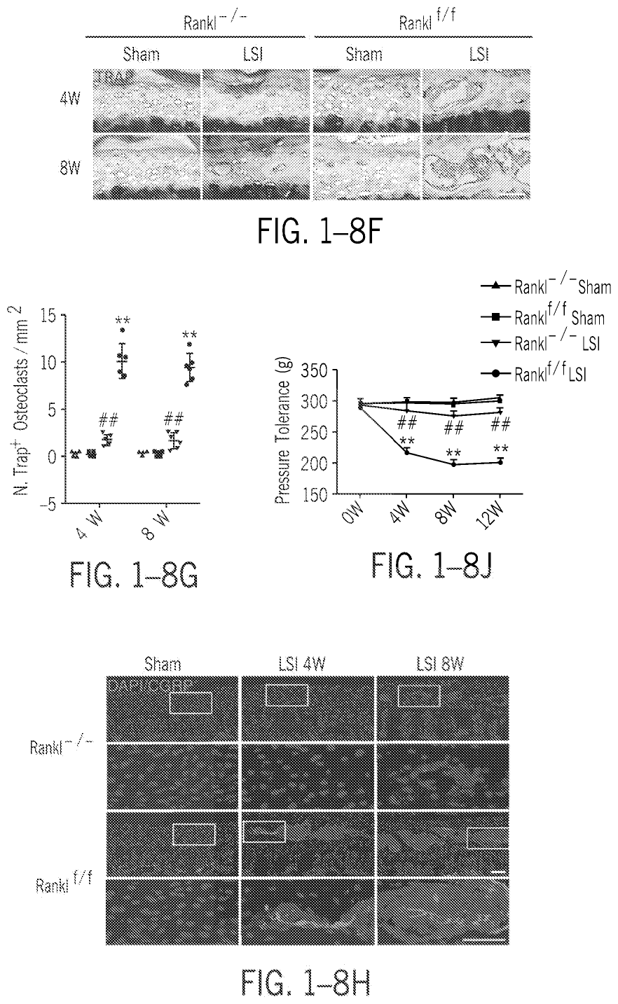

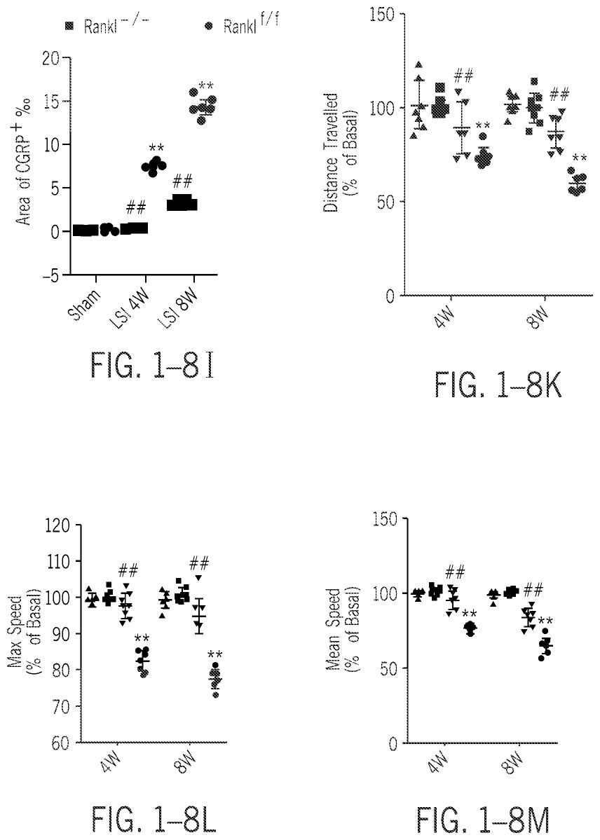

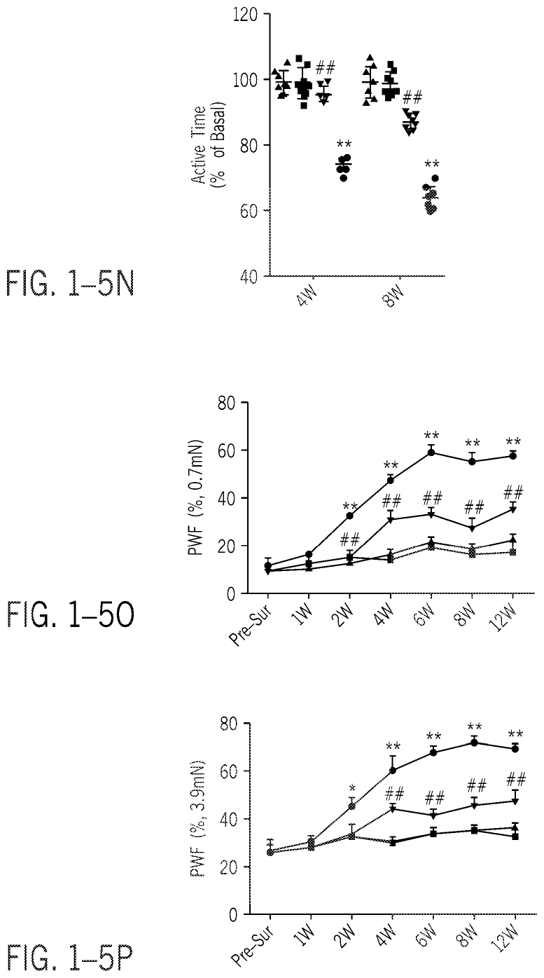

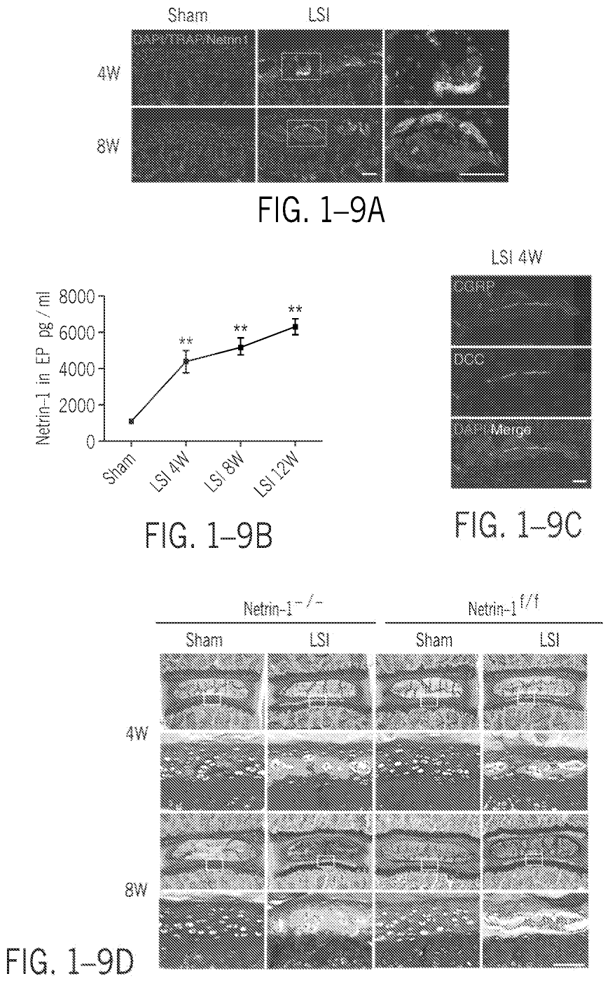

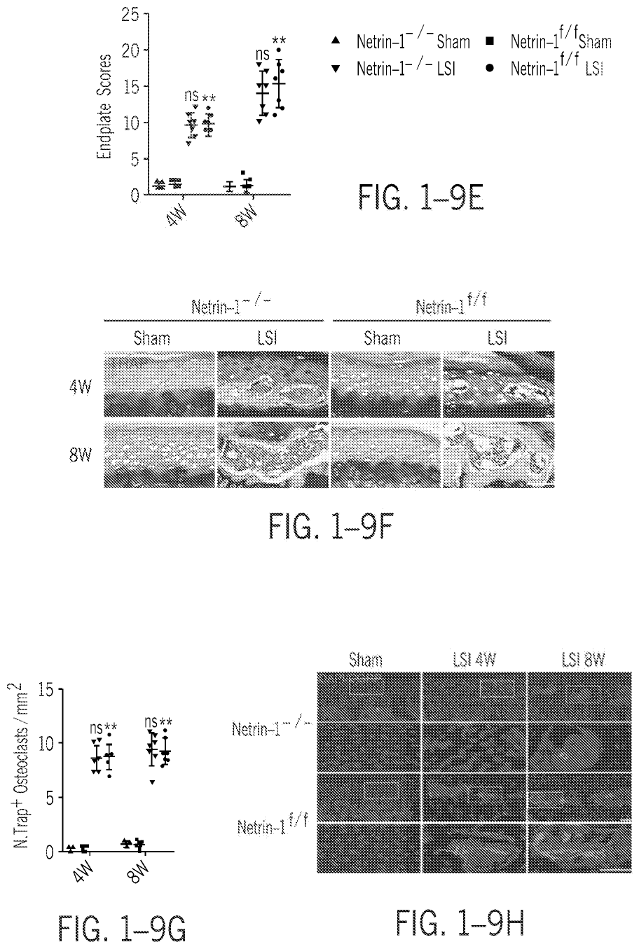

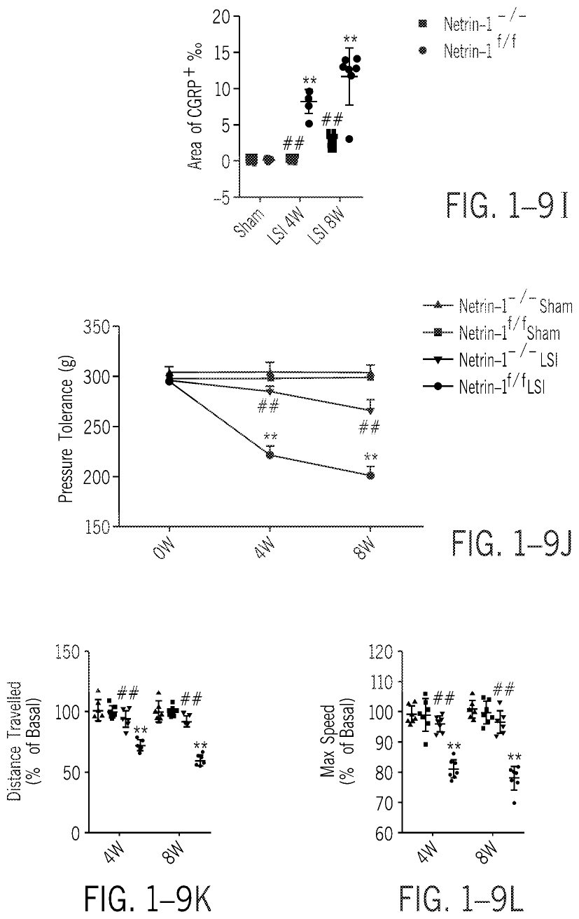

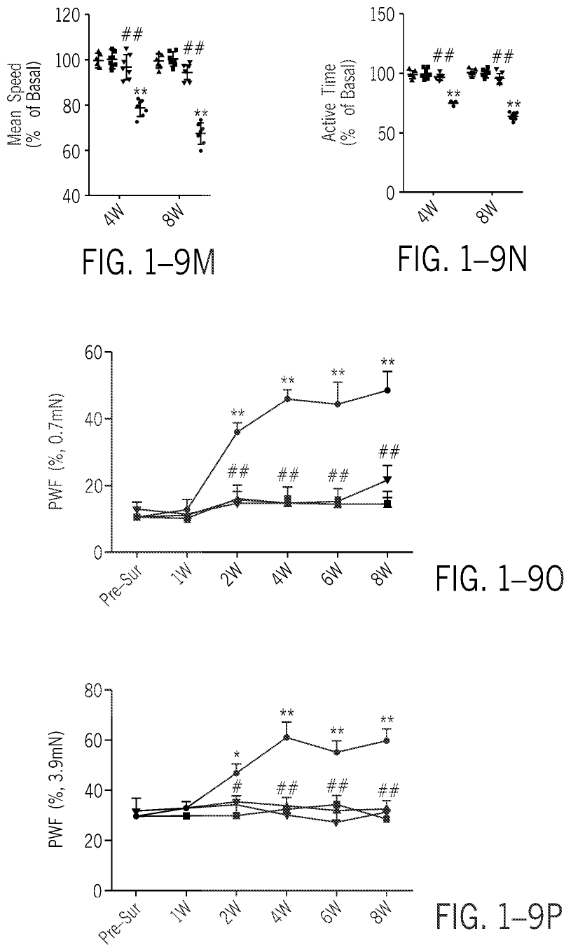

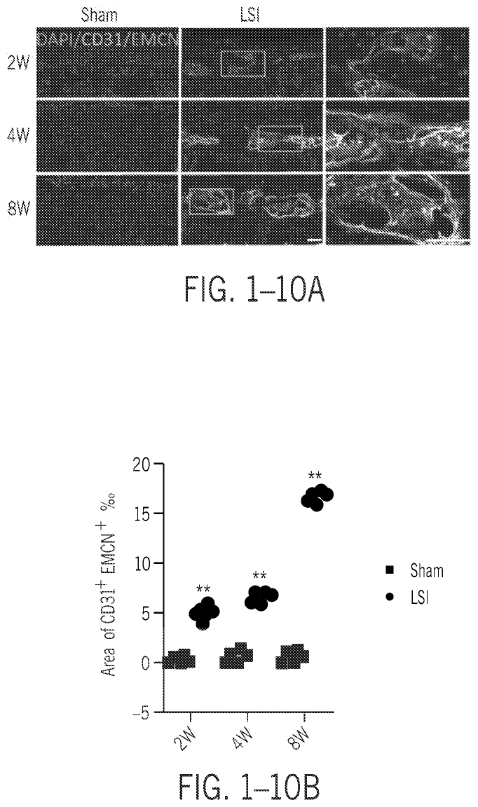

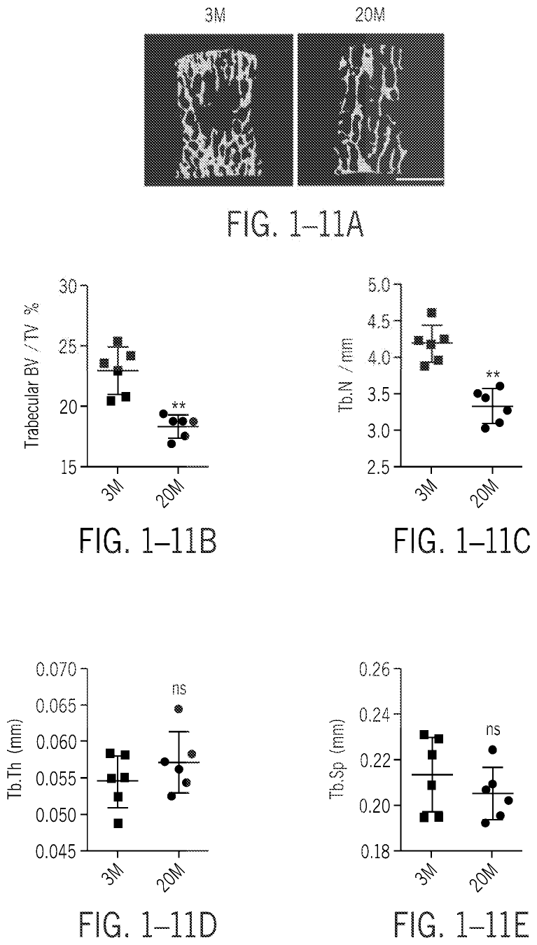

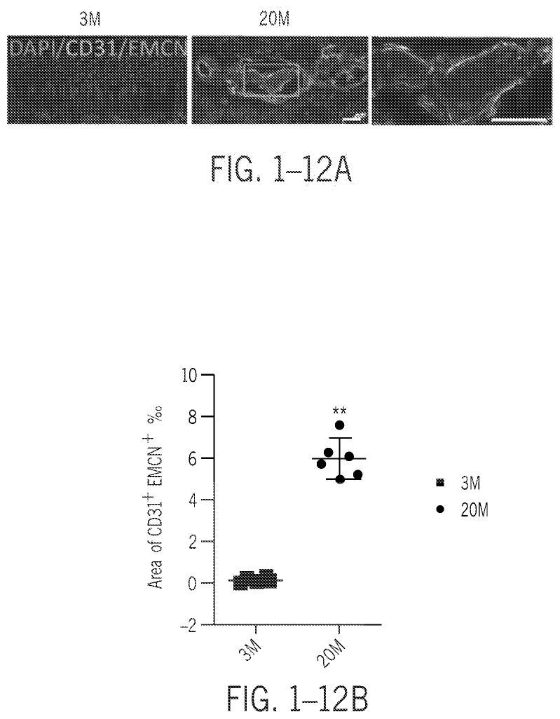

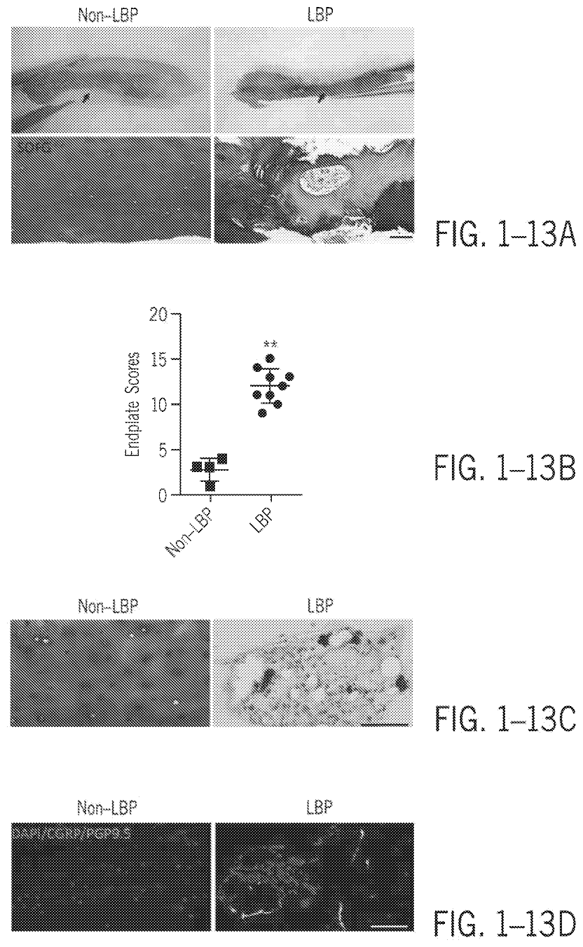

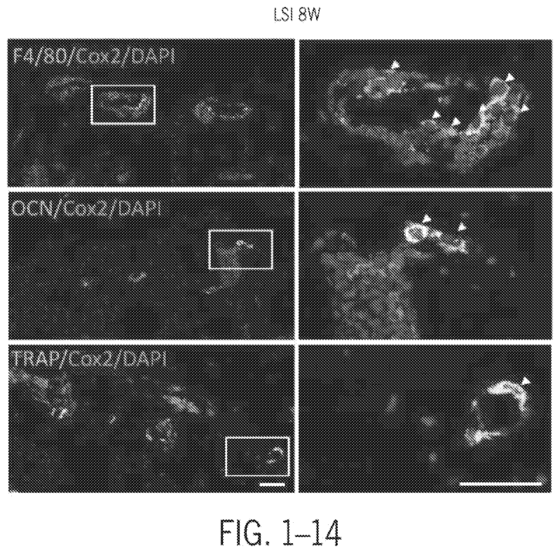

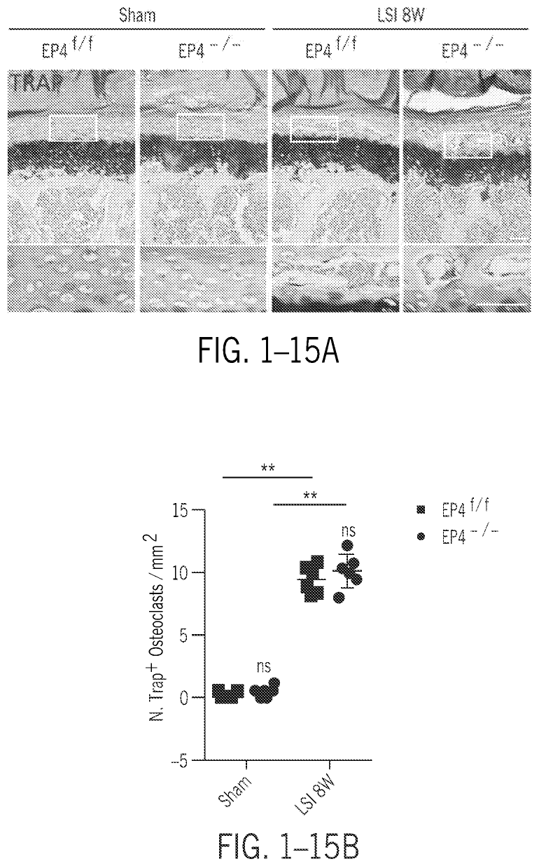

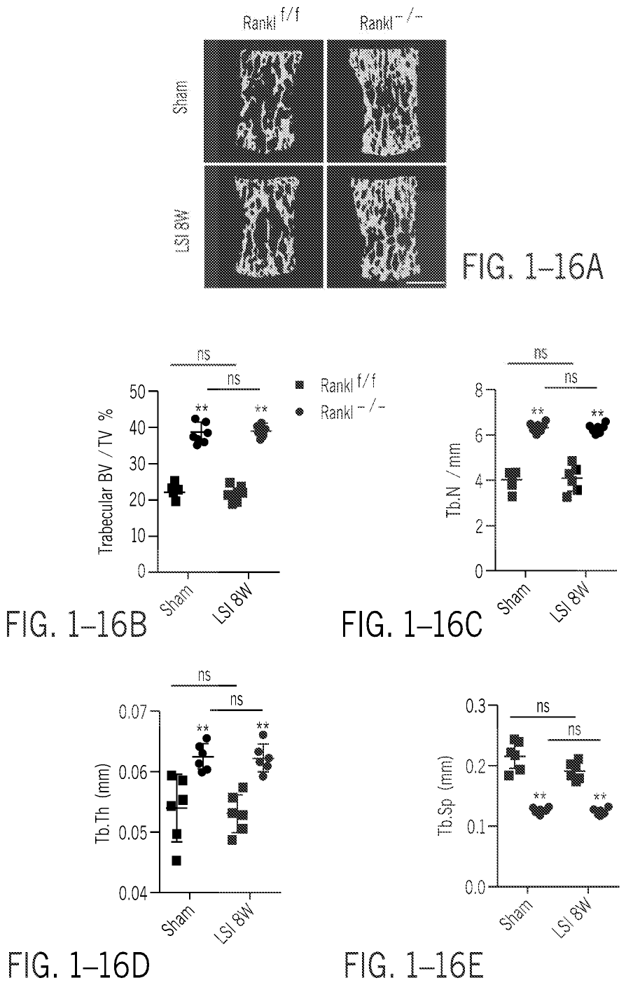

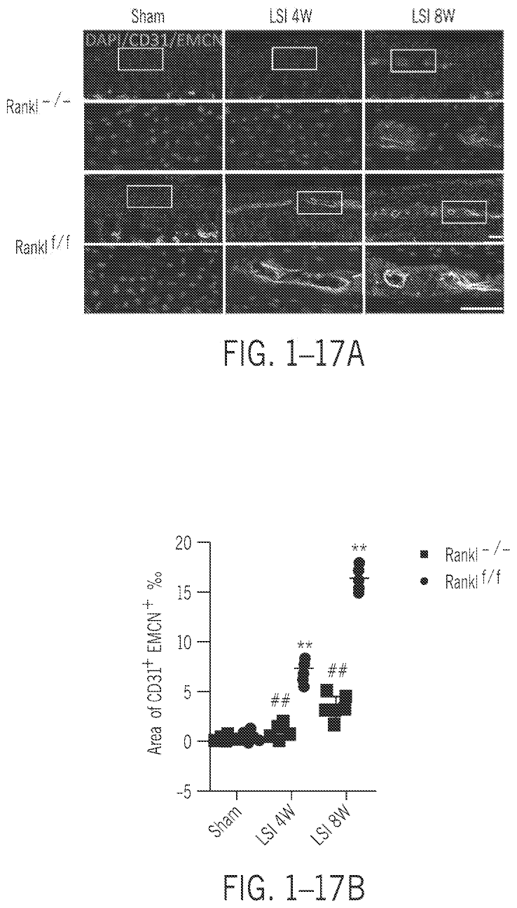

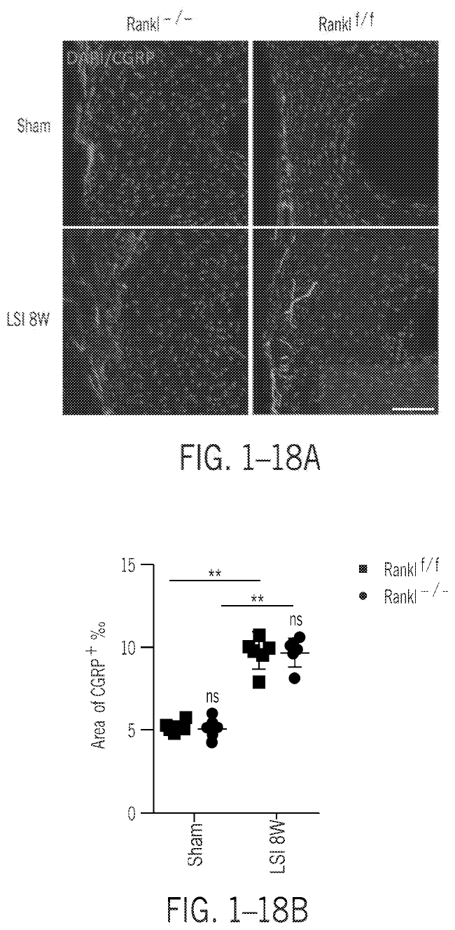

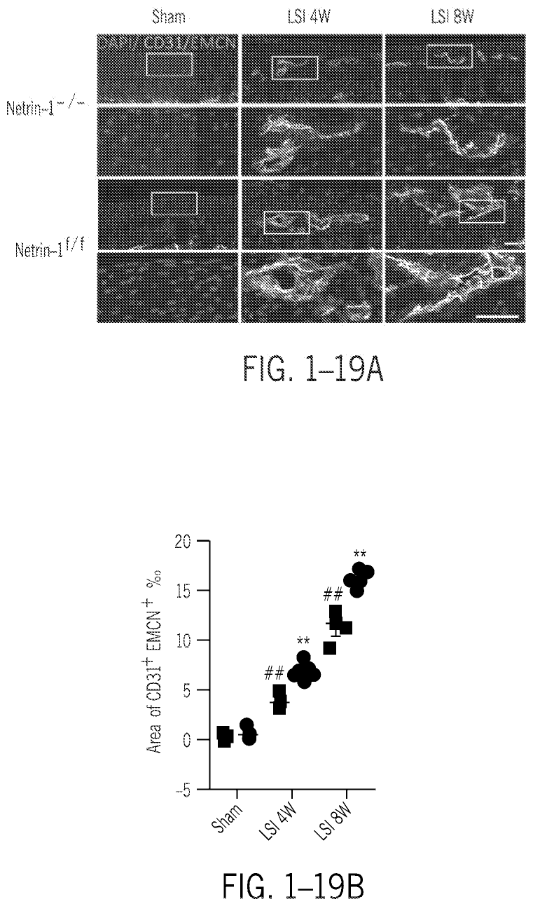

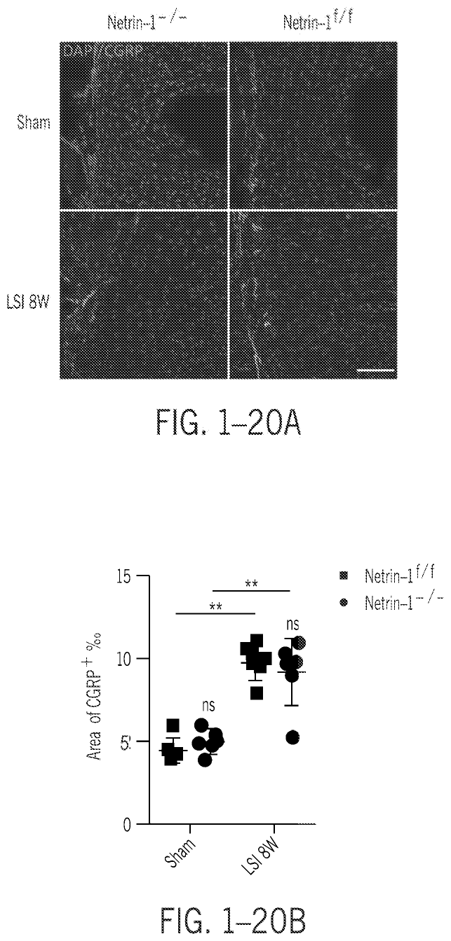

Lumbar spine instability (LSI) was established in mice as a spine degeneration model for spine pain behavior testing. Bian et al., 2016; Ariga et al., 2001; and Miyamoto et al., 2001. The vocalization threshold was first measured in response to force applied on the L4/L5 disc region. Pressure tolerance decreased significantly at 4, 8, and 12 weeks after LSI surgery relative to mice that underwent sham surgery (FIG. 1-1A), indicating the development of low back pressure hyperalgesia. Then, spontaneous activity was monitored to indicate the potential effect of spinal pain, including distance traveled, maximum speed of movement, mean speed of movement, and active time per 24 hours, though they are not specific for spinal pain behaviors. The results revealed that each measure of spontaneous activity decreased significantly at 4 and 8 weeks after LSI surgery relative to sham surgery (FIG. 1-1B-FIG. 1-1E). Moreover, mechanical hyperalgesia of the hind paw as referred pain was assessed by performing von Frey analysis, as a secondary indicator of symptomatic LBP. Paw withdraw frequency increased significantly from 2 to 12 weeks after LSI surgery (FIG. 1-1F, FIG. 1-1G). However, no response to the straight leg raising test was observed when recording the number of vocalizations during 5 leg stretch-and-lifts in either LSI or sham surgery mice. This observation indicates that nerve root compression is not involved in the hyperalgesia developed after LSI surgery. The results of these pain behavior tests suggest that spine instability induces the development of hyperalgesia.

In parallel, symptomatic LBP during aging in these behavior tests was evaluated. Similarly, the threshold of pressure tolerance (FIG. 1-1H) and spontaneous activity (FIG. 1-1I-FIG. 1-1K) decreased significantly in aged mice (age 20 months) relative to young mice (age 3 months). The mechanical hyperalgesia of the hind paw increased significantly in aged mice relative to young mice (FIG. 1-1L, 1-1M). Together, these data indicate that, as in the LSI mouse model, aging also induces spine hyperalgesia.

### 1.3.2 Sensory Innervation in Endplates in LS1 and Aged Mouse Models

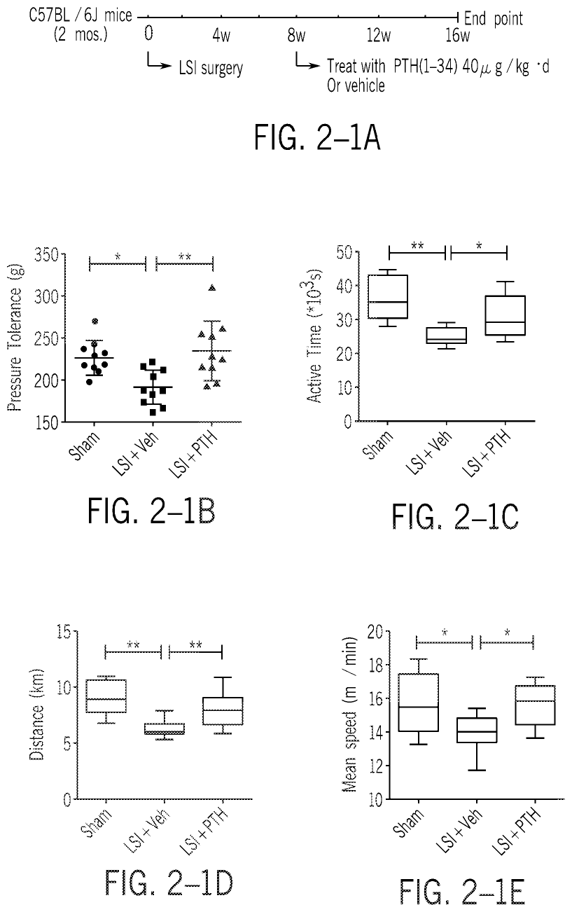

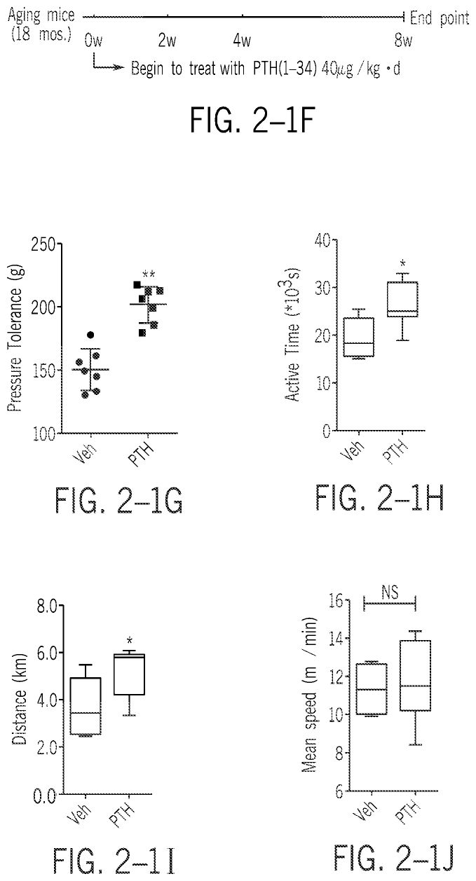

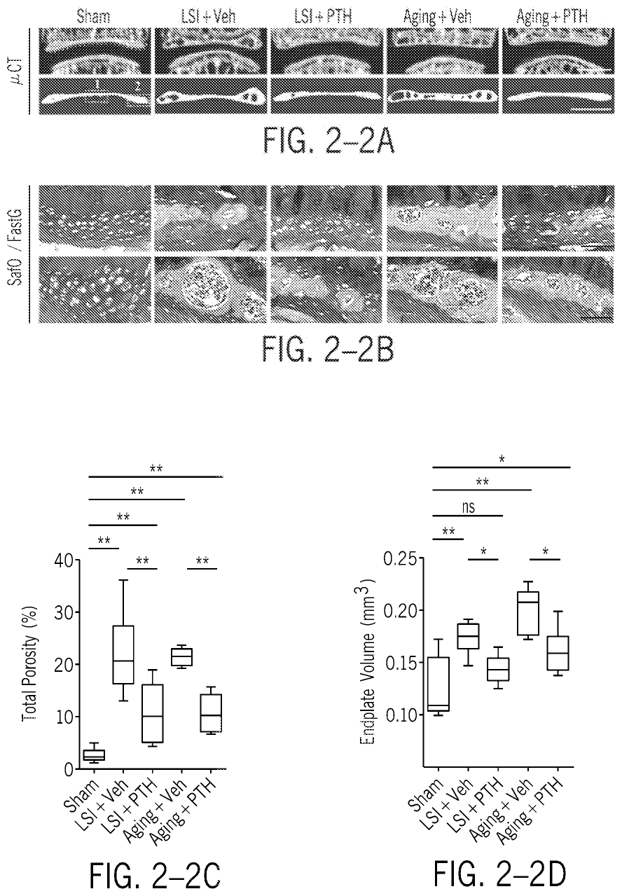

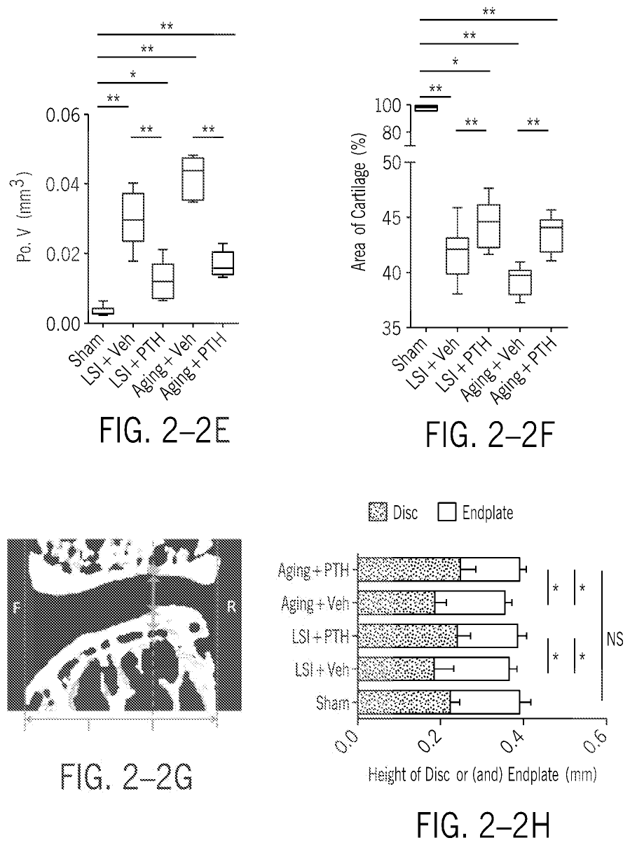

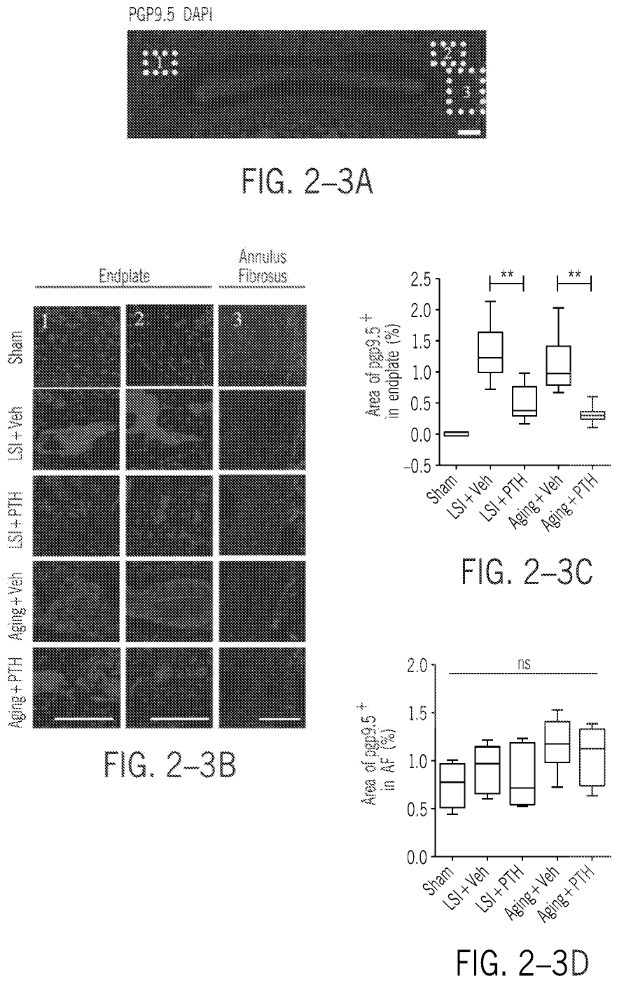

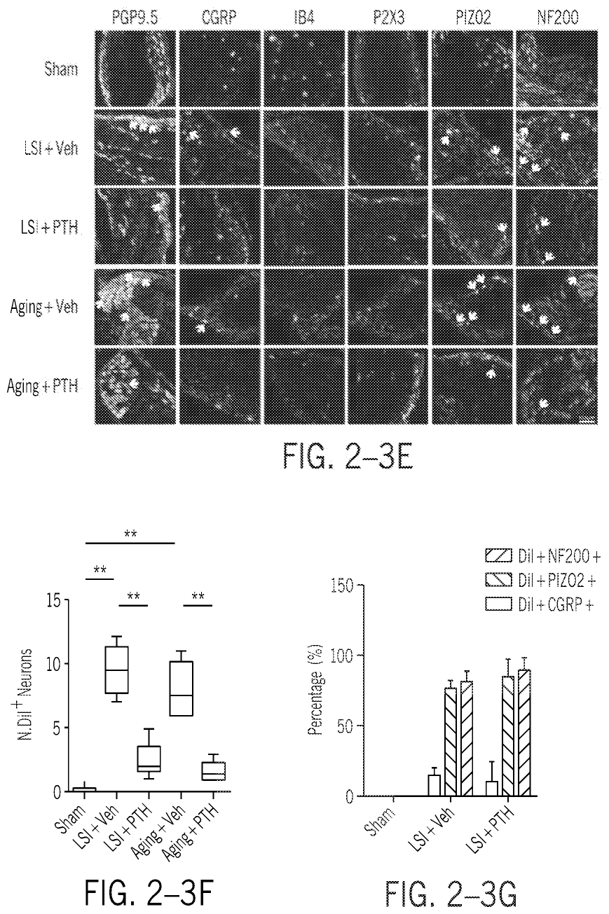

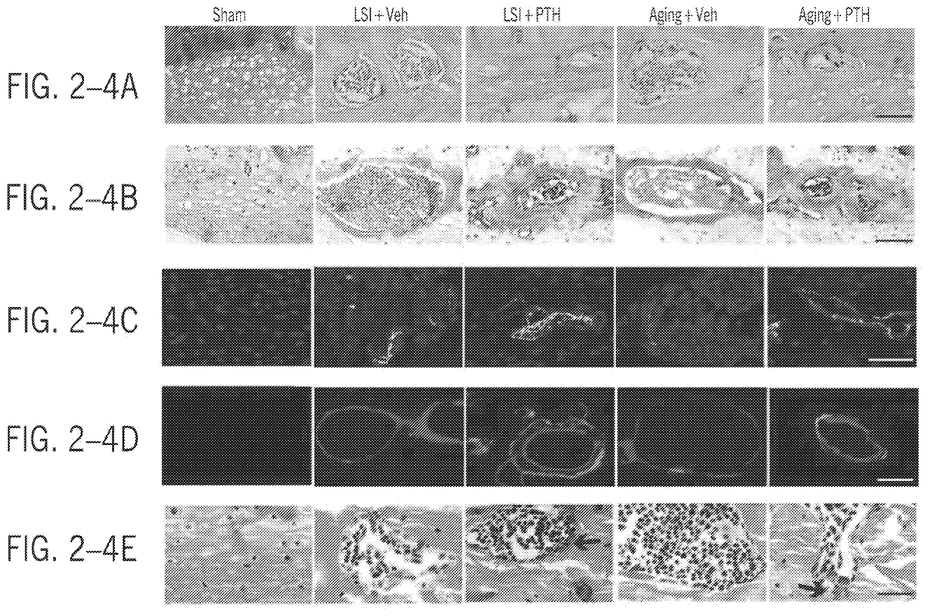

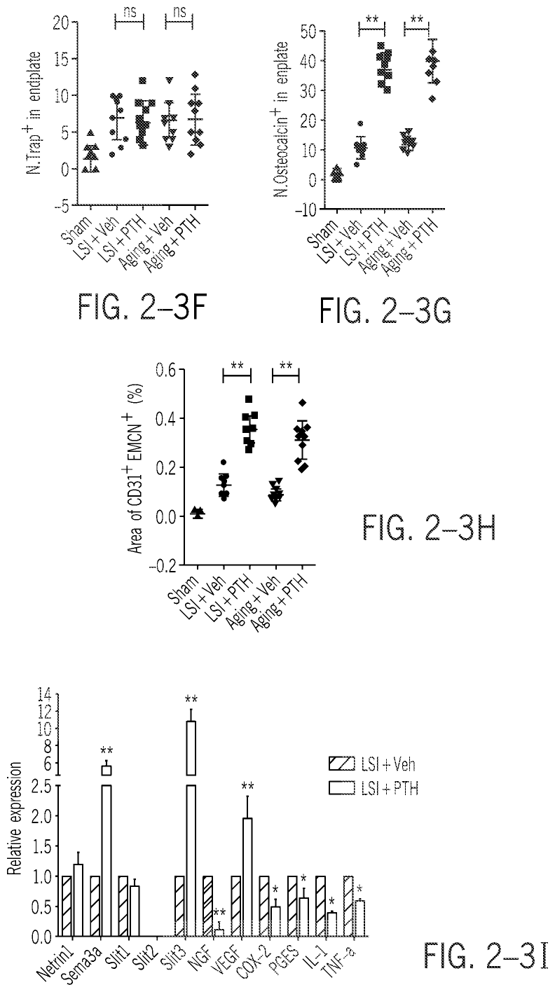

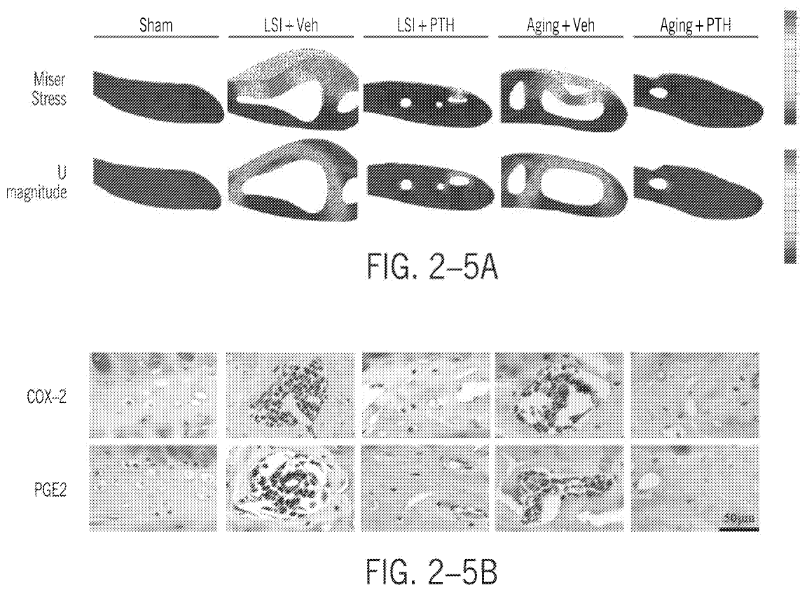

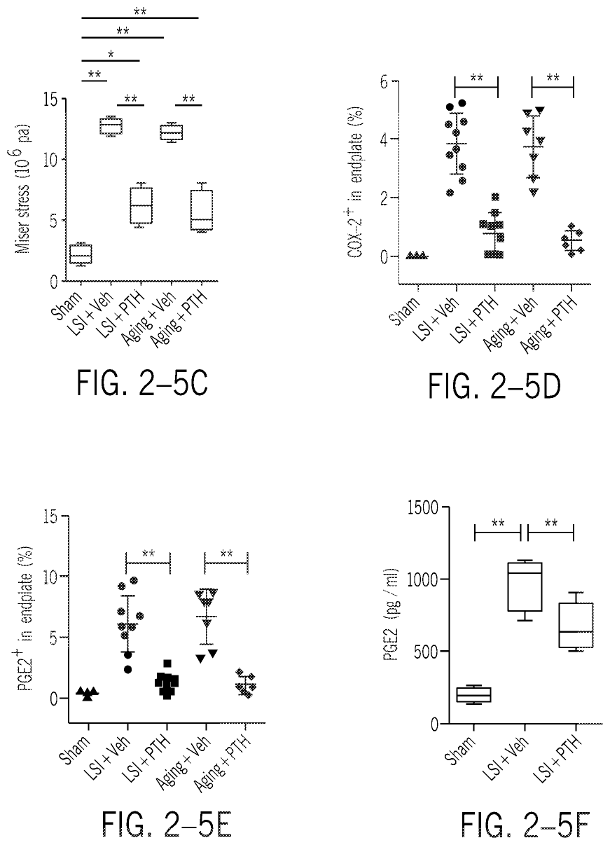

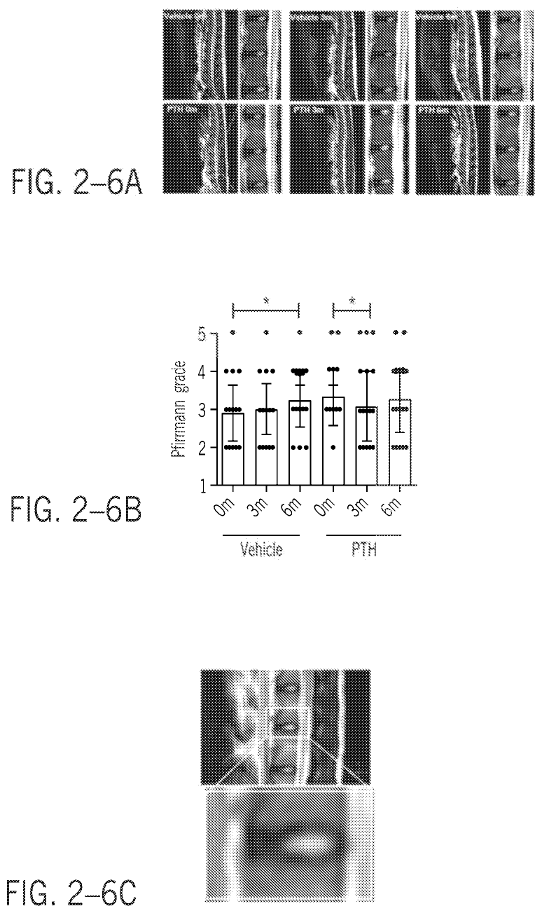

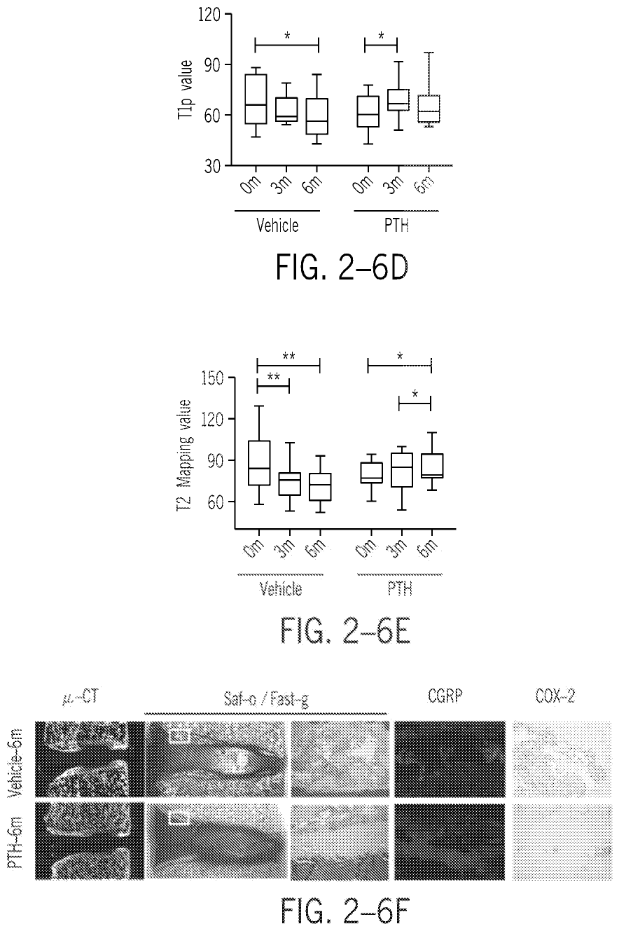

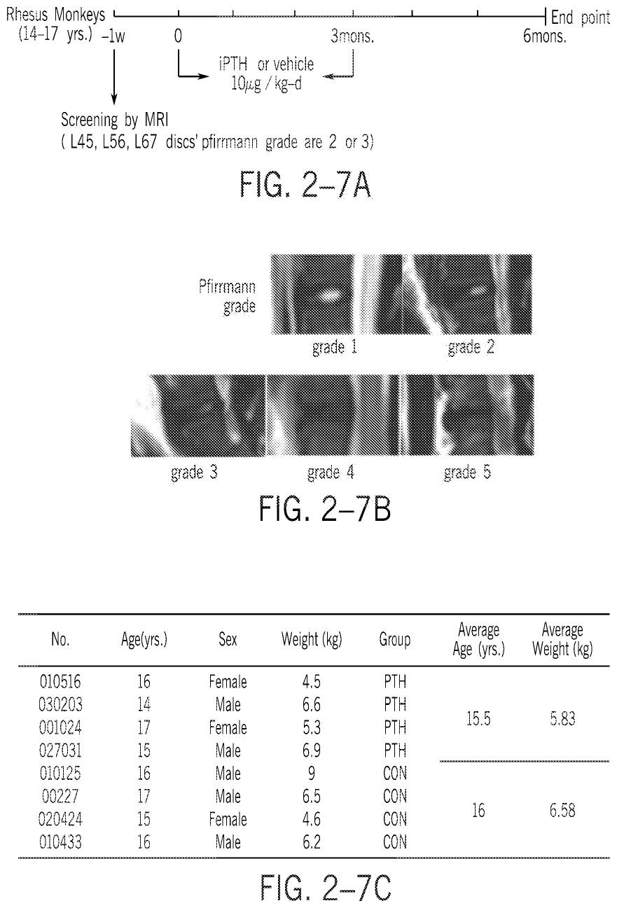

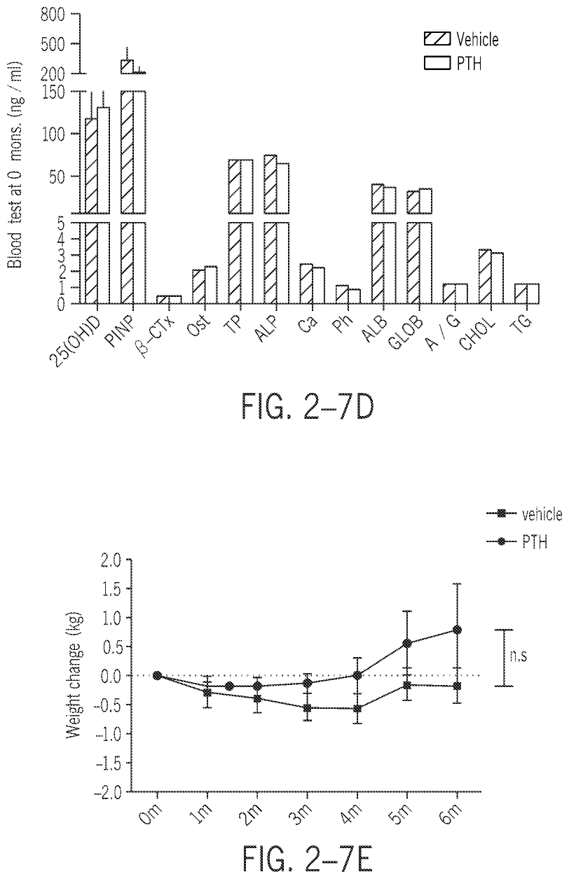

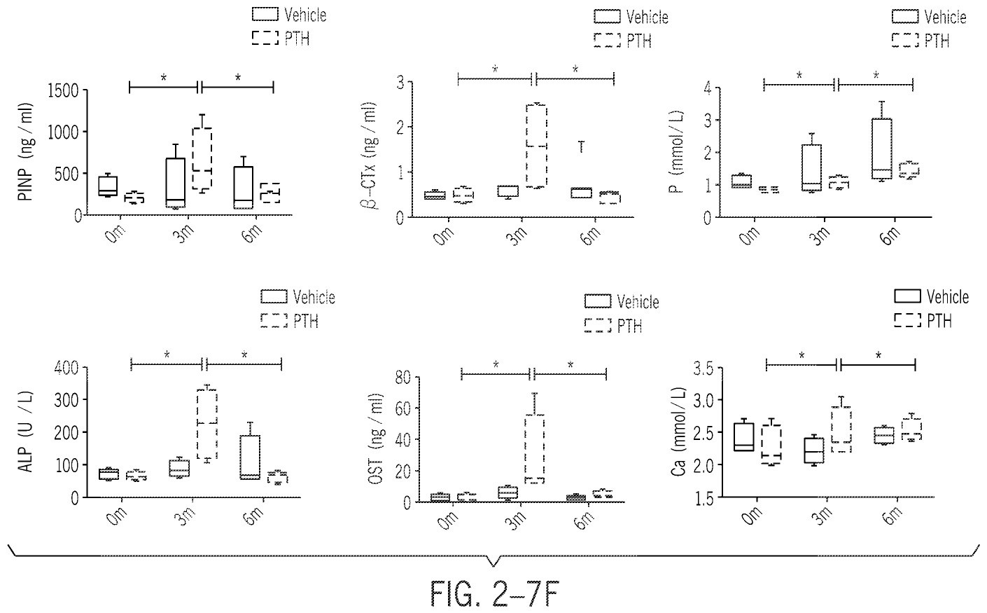

An increase in osteoclasts at the onset of endplate sclerosis was previously shown. Bian et al., 2016. Therefore, the potential role of osteoclasts in the sensory innervation of endplates was evaluated in this study. Tartrate-resistant acid phosphatase (TRAP) staining demonstrated that the number of TRAP⁺ osteoclasts in endplates increased significantly at 2 weeks after LSI and remained at a high level until 8 weeks after LSI surgery (FIG. 1-2A, FIG. 1-2B). Large bone marrow cavities were generated in sclerotic endplates by osteoclastic bone resorption in mice after LSI surgery, whereas, the cartilaginous endplates were maintained in sham surgery mice (FIG. 1-2A). Immunofluorescent staining revealed that the significant increase of CGRP, the marker of peptidergic nociceptive C nerve fibers in the porous endplates, began at 2 weeks and continued to increase until 8 weeks after LSI surgery (FIG. 1-2C, FIG. 1-2D), but there were no detectable CGRP⁺ nerves in the endplates of sham surgery mice (FIG. 1-2C, FIG. 1-2D). Interestingly, the nociceptive nerve fibers were localized primarily adjacent to the bone surface (FIG. 1-2C). The co-staining of PGP9.5, the broad marker of nerve fibers with CGRP, further validated the nociceptive innervation of the endplates after LSI surgery (FIG. 1-2E). However, the nonpeptidergic subtype of IB4⁺ C nerve fibers was not detected in either LSI or sham surgery mice (FIG. 1-2F), suggesting CGRP⁺ nerve fibers as the primary nociceptive C nerve fibers in the endplates. Importantly. CD3⁺IEMCN⁺ blood vessels were also growing into the porous endplates after LSI surgery, along with the sensory innervation of the endplates (FIG. 1-10A, FIG. 1-10B).

To determine whether spine degeneration during aging could induce sclerosis and sensory innervation in vertebral endplates, the caudal endplates of L4/5 from aged mice and young mice were analyzed. The porosity of endplates in aged mice increased significantly relative to young mice, as determined by 3-dimensional microcomputed tomography (μCT) analysis (FIG. 1-3A-FIG. 1-3C). The μCT analysis of vertebral trabecular bone demonstrated that the trabecular bone volume/total volume (BV/TV) and trabecular bone number (Tb.N) of L5 vertebrae decreased significantly in 20-month-old mice relative to 3-month-old mice, while the trabecular bone thickness (Tb.Th) and trabecular bone separation distribution (Tb.Sp) did not change significantly (FIG. 1-11A-FIG. 1-11E) Safranin O and fast green staining demonstrated that the green-stained bone matrix surrounded the cavities in endplates of aged mice (FIG. 1-3D, top and middle), suggesting endochondral ossification. Endplate scores, which are a histologic assessment of pathological changes such as bony sclerosis, structure disorganization, and neovascularization, were significantly higher in aged mice than that in young mice (FIG. 1-3E). Interestingly, high levels of TRAP⁺ osteoclasts were observed in the endplates of aged mice, whereas TRAP⁺ osteoclasts were rarely detected in the endplates of young mice (FIG. 1-3C, bottom and FIG. 1-3F). Immunostaining of CGRP showed increased aberrant innervation of peptidergic nociceptive C nerve fibers in the porous endplates of aged mice (FIG. 1-3G and FIG. 1-3H) The co-staining of PGP9.5 and CGRP further confirmed that the endplates were innervated by nociceptive nerve fibers (FIG. 1-3I). Similar to the findings in LSI mice, CD31⁺EMCN⁺ blood vessels were detected in the endplates, along with CGRP⁺ nerve fibers during aging (FIG. 1-12A, FIG. 1-12B), indicating active ossification of the endplates.

To examine the potential involvement of sclerosis and sensory innervation of the endplates with pain behavior, the pathological changes in the endplates of the lower lumbar spines from patients with or without LBP history was evaluated. Severe endplate lesions were observed in patients with a history of frequent LBP, whereas the cartilaginous structure was preserved in patients without a history of frequent LBP, despite disc herniation (FIG. 1-13A). The increased endplate scores were also observed in patients with a history of frequent LBP (FIG. 1-13B). The patients with the history of frequent LBP, however, are older than the ones without the history of frequent LBP (Table 1).

TABLE 1
Information for the human samples

| | No-Low Back Pain | Frequent Low Back Pain |
|---|---|---|
| Sample Size | 4 | 9 |
| Age (Years) | 28.3 ± 3.5 | 56.6 ± 5.4 |
| Sex (Male/Female) | 3/1 | 5/4 |
| Body Mass Index | 22.5 ± 1.9 | 23.9 ± 2.4 |
| (BMI) | | |
| Disc Level | L4/5(3)/L5/S1(1) | L3/4(2)/L4/5(4)/L5/S1(3) |
| Endplate lesions | N/A | 8 |
| Pfirrmann grading | 1/3 | 4/5 |
| (grade 3/grade 4) | | |

TRAP staining showed that abundant TRAP⁺ osteoclasts localized at the bone surface in the sclerotic endplates (FIG. 1-13C). Immunofluorescence staining revealed that CGRP⁺PGP9.5⁺ nociceptive nerve fibers grown into the porous areas of sclerotic endplates of patients with LBP history (FIG. 1-13D). These results suggest that sensory innervation in sclerotic endplates is potentially related to spinal pain behavior.

### 1.3.3 Retrograde and Anterograde Tracing of Sensory Innervation

To demonstrate CGRP⁺ sensory innervation in endplates during spine degeneration, a retrograde tracing experiment in both LSI and aged mice was conducted. The red fluorescent tracer, Dil, was injected in the left part of the caudal endplates of L4/5 in mice at 8 weeks after LSI surgery (FIG. 1-4A). The T12-L6 dorsal root ganglions (DRGs) in both sides were harvested at 1 week after injection to calculate the number of Dil⁺ neurons. It was observed that Dil was retrograded mainly to the left T13-L3 DRGs, especially the left L1 and L2 DRGs in LSI mice, whereas no Dil⁺ neurons were detected in the T12-L6 DRGs of sham surgery mice (FIG. 1-4B, FIG. 1-4C). Immunofluorescent staining of the DRG sections demonstrated that Dil in the left L1 and L2 DRGs was co-localized mainly with CGRP⁺ but not IB4⁺ neurons in LSI mice (FIG. 14D, FIG. 1-4E).

The anterograde tracing experiment was performed by labeling the L1 and L2 DRG neurons in both sides with injection of Dil at 8 weeks after LSI or sham surgery. Abundant Dil-labeled sensory nerves were seen in the porous areas of endplates of L4/5 in LSI mice, but not in sham surgery mice (FIG. 1-4F, FIG. 1-4G). Similarly, the innervation of Dil-labeled sensory nerves in the porous areas of endplates of aged mice was also observed in the anterograde tracing experiment (FIG. 1-4H, FIG. 1-4I). Taken together, these findings suggest nociceptive innervation in the sclerotic endplates of LSI and aged mice.

### 1.3.4 PGE2′EP4 Signaling Mediates Spinal Hypersensitivity

To elucidate the signaling mechanism of endplate sclerosis-mediated pain behavior, the expression of several inflammatory cytokines in the endplates of LSI and sham surgery mice was examined by using quantitative real-time polymerase chain reaction (qRT-PCR). It was found that messenger ribonucleic acid levels of prostaglandin E synthase (PGES), cox2, interleukin (IL)-1β, IL-17, IL-2, and tumor necrosis factor (TNF)-α increased significantly in the lumbar endplates at 4 weeks after LSI relative to sham surgery, especially PGES (FIG. 1-5A). Immunostaining further confirmed a significant increase in cox2 in the endplates at 4 and 8 weeks after LSI surgery (FIG. 1-5B, top). The increases of PGES, cox2, and IL-10 can contribute to the synthesis of PGE2 in the endplates, which was validated by immunostaining (FIG. 1-5B, bottom) and enzyme-linked immunoabsorbent assay (ELISA) (FIG. 1-5C). The increase of PGE2 in endplates peaked at 4 weeks and remained high at 8 and 12 weeks after LSI surgery (FIG. 1-5C). To explore the potential source of PGE2 in porous endplates, the co-immunostaining for cox2 with F4/80, cox2 with osteocalcin (OCN), and cox2 with TRAP were conducted respectively. The results demonstrated that the COX2 was co-localized with F4/80⁺, some OCN⁺, and a few TRAP⁺ cells (FIG. 1-14). These data showed that the accumulated PGE2 in porous endplates was derived from different types of cell. Immunostaining showed that EP4 was expressed in newly innervated CGRP⁺ nerve endings in the endplates of LSI mice (FIG. 1-5D). Notably, the proportion of CGRP⁺EP4⁺ neurons relative to CGRP⁺ neurons in L2 DRGs was significantly greater in LSI mice relative to sham surgery mice (FIG. 1-5E, FIG. 1-5F). Interestingly, the sodium channel Naᵥ 1.8 was also expressed in newly innervated CGRP⁺ nerve ending in the endplates of LSI mice, as demonstrated by immunostaining (FIG. 1-5G). Moreover, the proportion of CGRP⁺Naᵥ1.8⁺ neurons relative to CGRP⁺ neurons in L2 DRGs increased significantly at 4 and 8 weeks after LSI surgery (FIG. 1-5H, FIG. 1-5I).

To examine the potential role of PGE2/EP4 in pain transduction, a sensory neuron specific EP4 knockout mice (Avil^{Cre}; EP4^{flox/flox}, named EP4 mice) was generated. TRAP staining demonstrated that there was no significant difference in the number of TRAP⁺ osteoclasts in endplates between EP4^{f/f} and EP4^{-/-} mice of sham surgery group or LSI surgery group (FIG. 1-15A, FIG. 1-15B). Asante NaTRIUM Green 2 acetoxymethyl (ANG-2 AM), a sodium indicator, was loaded into the DRG neurons to detect the real-time sodium influx. Interestingly, PGE2 significantly stimulated the enhancement of the fluorescent intensity in neurons (FIG. 1-6A, left and FIG. 1-6B), indicating increased sodium influx. Importantly, this effect was abolished in the DRG neurons of EP4^{-/-} mice (FIG. 1-6A, right and FIG. 1-6C). To determine the mechanism by which PGE2 induces sodium influx, whether PGE2 can activate the cyclic adenosine monophosphate (cAMP) pathway in sensory neurons was examined. Western blot and fluorescent staining demonstrated that PGE2-induced cAMP production activates protein kinase A (PKA) and cAMP response element binding (CREB) protein, and the activation was abrogated by PKA inhibitor or EP4^{-/-} (FIG. 1-6D-FIG. 1-6F). Further, PGE2-induced sodium influx was ablated by PKA inhibitor or small interfering ribonucleic acid (siRNA) for Naᵥ 1.8 (FIG. 1-6G, first to third columns and FIG. 1-6H-FIG. 1-6J), and cAMP rescued sodium influx in EP4^{-/-} mice (FIG. 1-6G, fourth column and FIG. 1-6K). These results demonstrate that PGE2 activates EP4 in sensory, neurons to induce sodium influx of Naᵥ 1.8 through cAMP signaling with implications for pain transduction.

To evaluate whether the PGE2/EP4 pathway in sensory fibers is associated with spinal pain behavior, pain behavior tests, including pressure tolerance, spontaneous activity, and von Frey analysis, were performed. The threshold of pressure tolerance in response to pressure stimulation was lower in EP4^{-/-} mice relative to EP4^{f/f} mice after LSI surgery (FIG. 1-7A). Analysis of spontaneous activity revealed that the distance traveled, maximum speed, mean speed, and activity time per 24 hours were significantly preserved in EP4^{-/-} mice relative to EP4^{f/f} f mice after LSI surgery (FIG. 1-7B-FIG. 1-7E). Consistently, mechanical hyperalgesia of the hind paw also was ameliorated in EP4^{-/-} mice relative to EP4^{f/f} mice after LSI surgery (FIG. 1-7F, FIG. 1-7G).

### 1.3.5 Decreased Osteoclast Attenuated Sensory Innervation and Pain

To evaluate whether CGRP⁺ sensory innervation in the endplates is induced by osteoclasts, Dmp1-Cre mice were bred with osteocyte-derived receptor activator of nuclear factor kappa-B ligand (Rankl) floxed mice to generate Dmp1^{Cre}: Rankl^{flox/flox} mice (named Rankl^{-/-} mice) to knock out Rankl specifically in DMP1⁺ osteocytes. Deficiency of Rankl in osteocytes leads to a decrease in osteoclast number and a severe osteopetrotic phenotype. Nakashima et al., 2011: Xiong et al., 2011. The trabecular BV/TV, Th.N, and Tb.Th increased and Tb.Sp decreased significantly in RANKL^{-/-} mice relative to RANKL^{f/f} mice in μCT analysis (FIG. 1-16A-FIG. 1-16F), indicating the osteopetrotic vertebrae in Rankl-mice. The sclerosis of endplates was delayed significantly in Rankl^{-/-} mice relative to their age-matched wild-type (WT) littermates (Rankl^{f/f} mice) after LSI surgery, as shown by decreases in the porosity and trabecular separation of endplates by μCT analysis (FIG. 1-8A-FIG. 1-8C). The delayed sclerosis of endplates in Rankl^{-/-} mice was further validated by Safranin O and fast green staining, with less porous areas and significant lower endplate scores (FIG. 1-8D, FIG. 1-8E). The number of TRAP⁺ osteoclasts decreased significantly in endplates at 4 and 8 weeks after LSI surgery in Rankl^{-/-} mice relative to Rankl^{f/f} mice (FIG. 1-8F, FIG. 1-8G). Notably, immunostaining showed that instability-induced CGRP⁺ sensory innervation in the endplates was inhibited in Rankl^{-/-} mice (FIG. 1-8H, FIG. 1-8I). Although the density of CGRP⁺ sensory nerve fibers increased slightly at 8 weeks after LSI surgery in Rankl^{-/-} mice, it was still significantly lower than that of Rankl^{f/f} mice (FIG. 1-8H, FIG. 1-8I), indicating that osteoclast activity was associated with CGRP⁺ sensory innervation in the endplates. Moreover, the sprouting of CD31⁺EMCN⁺ blood vessels in the endplates was also significantly inhibited in Rankl^{-/-} mice relative to Rankl^{f/f} mice (FIG. 1-17A, FIG. 1-17B).

To examine whether osteoclast activity-induced sensory innervation in endplates is associated with spinal pain behavior, pain behavior tests, including pressure tolerance, spontaneous activity, and von Frey analysis, were performed. The threshold of pressure tolerance was significantly lower in Rankl^{-/-} mice relative to Rankl^{f/f} mice after LSI surgery (FIG. 1-8J). Analysis of spontaneous activity revealed that the distance traveled, maximum speed, mean speed, and activity time per 24 hours were significantly preserved in Rankl^{-/-} mice relative to Rankl^{f/f} mice after LSI surgery (FIG. 8K-FIG. 8N). Consistently, mechanical hyperalgesia of the hind paw was also ameliorated in Rankl^{-/-} mice relative to Rankl^{f/f} mice after LSI surgery (FIG. 1-8O, FIG. 1-8P). Nerve innervation in the annulus fibrosus (AF) has been implicated in back pain development. Ohtori et al., 2015. CGRP⁺ sensory innervation was observed in the AF of LSI mice. There is no significant difference in the density of newly innervated sensory nerves between Rankl^{-/-} mice and Rankl^{f/f} mice (FIG. 1-18A-FIG. 1-18B). Taken together, these results indicate that sensory innervation in vertebral endplates is associated with osteoclast activity and pain behavior.

### 1.3.6 Knockout of Netrin-1 Abrogated Sensory Innervation and Pain

Netrin-1 is an important axon guidance factor to attract nerve protrusion. Moore et al., 2012; Serafini et al., 1996: and Hand and Kolodkin, 2017. It was previously reported that osteoclasts can secret Netrin-1 to attract sensory nerve growth. Zhu et al., 2019. Immunostaining demonstrated that TRAP⁺ osteoclasts were the source of Netrin-1 in sclerotic endplates after LSI surgery (FIG. 1-9A). The level of Netrin-1 in lower lumbar endplates increased gradually at 4, 8, and 12 weeks after LSI surgery relative to sham surgery mice, as indicated by ELISA (FIG. 1-9B). Deleted in colorectal cancer (DCC) is identified as the receptor that mediates Netrin-1-induced neuronal sprouting. Forcet et al., 2002; Shu et al., 2000. Immunostaining revealed that DCC was co-localized with CGRP⁺ sensory nerve fibers in endplates after LSI surgery (FIG. 1-9C).

To determine whether osteoclast-derived Netrin-1 is responsible for sensory innervation, Trap-Cre mice were cross-bred with Netrin-1 floxed mice to knockout Netrin-1 in TRAP⁺ lineage cells (Trap^{Cre}; Netrin-1^{flox/flox}, named Netrin-1^{-/-} mice). Safranin O and fast green staining demonstrated that the instability-induced sclerosis of endplates was not different in Netrin-1^{-/-} mice compared with their age-matched littermates (herein, Netrin-1^{f/f} mice) (FIG. 1-9D), as evidenced by endplate scores (FIG. 1-9E). TRAP staining showed similar numbers of osteoclasts in the endplates of Netrin-1^{-/-} mice and Netrin-1^{f/f} mice after LSI surgery (FIG. 1-9F, FIG. 1-9G), suggesting that Netrin-1 is not involved in endplate sclerosis. The density of CGRP® nociceptive nerves did not increase in Netrin-1^{-/-} mice, although the number of TRAP⁺ osteoclasts significantly increased in the endplates of Netrin-1^{-/-} mice after LSI surgery (FIG. 1-9H, 1-9I). Moreover, the sprouting of CD31⁺EMCN⁺ blood vessels in the endplates was significantly inhibited in Netrin-1^{-/-} mice relative to Netrin-1^{f/f} mice (FIG. 1-19A. FIG. 1-19B).

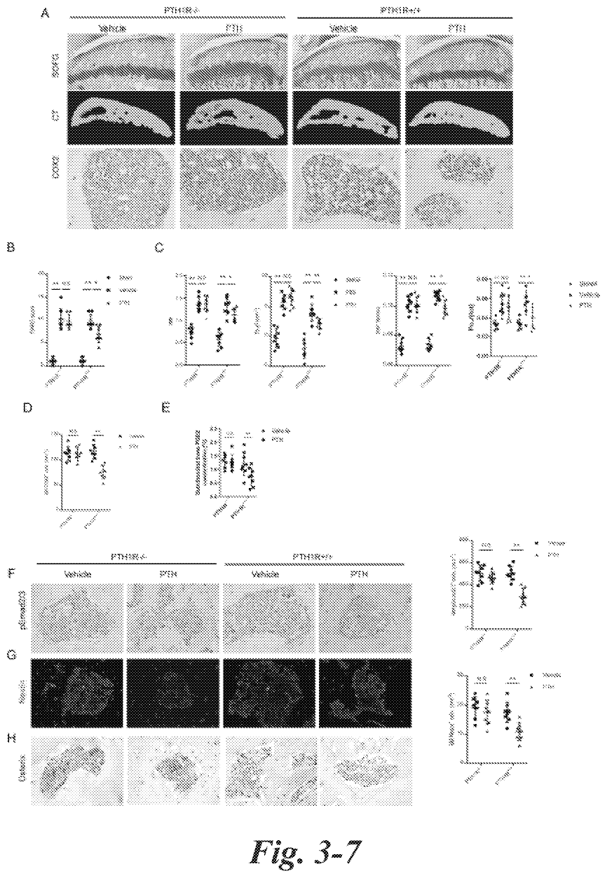

Pain behavior tests demonstrated that pressure hyperalgesia (FIG. 1-9J), loss of spontaneous activity (FIG. 1-9K-FIG. 1-9N), and mechanical hyperalgesia of the hind paw (FIG. 1-9O, FIG. 1-9P) were significantly lower at 4 and 8 weeks after LSI surgery in Netrin-1^{-/-} mice relative to Netrin-1^{f/f} mice. As in Rankl^{-/-} mice, CGRP⁺ sensory innervation in the AF did not decrease in Netrin-1^{-/-} mice (FIG. 1-20A, FIG. 1-20B). Together, these results indicate that osteoclast-derived Netrin-1 mediates sensory innervation in vertebral endplates responsible for spinal pain behavior.

### 1.4. Discussion

LBP is difficult to diagnose and treat because of limited knowledge about its source. Current treatments, including activity modification, physical therapy, and pharmaceutical agents aim to alleviate the pain, but not to modify the disease. Maher et al, 2017: Foster et al., 2018. Surgical treatment, such as disc replacement and lumbar fusion, are the most common final treatments. Efforts to understand the causes of LBP have focused largely on sensory innervation in the degenerative IVD. Garcia-Cosamalon et al., 2010. However, IVD degeneration is frequently asymptomatic. Importantly, endplates undergo sclerosis during aging and become porous, which is clinically associated with LBP. Lotz et al., 2013; Dudli et al., 2016; and Brown et al., 1997. It has previously been shown that aberrant mechanical loading induces sclerosis of the endplates with elevated osteoclast activity and activates excessive TGF-01 to recruit mesenchymal stromal cells. Bian, 2016. Here, elevated level of PGE2 and sensory innervation in the porous sclerotic endplates was observed. PGE2 could activate its receptor EP4 in the newly innervated sensory nerves to cause spinal pain. It was recently reported that sensory nerves can monitor bone density changes through the concentration of PGE2. Osteoblast-secreted PGE2 activates its receptor EP4 in the sensory nerves to tune down sympathetic tone for osteoblastic bone formation. Chen et al., 2019. The porosity of the sclerotic endplates resembles low bone mineral density to promote PGE2 concentration and sensory innervation causing pain. These results indicate that PGE2 is a key mediator of pain hypersensitivity and endplate sclerosis. These findings suggest that inhibition of endplate sclerosis to reduce sensory innervation or blocking the PGE2/EP4 pathway could ameliorate spinal pain behavior.

Sensory nerves and CD31⁺EMCN⁺ vessels appeared largely in the porous areas of the sclerotic endplates. Osteoclast resorption likely causes the porosity of sclerotic endplates. Osteoclastic lineage cells secrete Netrin-1 to induce CGRP⁺ sensory nerve fiber extrusion and innervation in the space generated by osteoclast resorption. In addition, Netrin-1 is suggested to be a potential pro-angiogenic factor that promotes endothelial cell migration and capillary-like tube formation. Park et al., 2004; Tu et al., 2015. It has been shown that pre-osteoclasts secrete platelet-derived growth factor-BB to induce angiogenesis coupled with osteogenesis during bone formation. Xie et al., 2014. Thus, osteoclast activity in the sclerotic endplates is the primary cause of sensory innervation and angiogenesis for LBP. Indeed, decreased osteoclasts in Rankl^{-/-} mice led to significantly decreased sensory innervation and angiogenesis in the endplates. Moreover, the density of sensory innervation and angiogenesis was reduced significantly by conditional knockout of Netrin-1 in TRAP⁺ osteoclastic cells. Therefore, osteoclast lineage cells instigate porosity of sclerotic endplates and spinal pain behavior.

The IVD and the endplate act in as a functional unit in the spine. A previous study revealed that aberrant mechanical loading induced hypertrophy of chondrocytes and calcification of the endplates, leading to an osteoclast-initiated remodeling sclerotic process. As a result, the expansion of mineralized endplates narrowed the intervertebral space and generated pathological static compression on the intervertebral disc, leading to its degeneration. Bian, 2016. The AF has been suggested as the main source of discogenic pain. In the physiological condition, only the outer third of the AF is innervated by sensory nerves, whereas in degenerative discs, the nerve can grow into the middle third, or even the inner third of the AF. Ohtori et al, 2015. In the current study, innervation of CGRP⁺ sensory fibers into the AF in WT mice after LSI surgery was observed, but the LSI-induced newly innervated sensory nerves in the AF were not decreased in Rankl^{-/-} and Netrin-1^{-/-} mice. On the other hand, during sclerosis of the endplates, osteoclast resorption generates porous areas and abundant sensory innervation along with angiogenesis. Importantly, the density of CGRP⁺ sensory nerve fibers in endplates and spinal pain behavior were significantly ameliorated in Rankl^{-/-} and Netrin-1^{-/-} mice after LSI surgery. These results indicate that osteoclast-induced sensory innervation in the sclerotic endplates is the primary source of nociceptors for spinal pain.

The sign of the hind paw mechanical allodynia is considered as the secondary indicator of spinal pain-associated behaviors. Several studies reported that the hind paw mechanical allodynia develops in low back pain animal models as the secondary hypersensitivity. Shuang et al., 2015; Kim et al., 2011; Millecamps et al., 2015: and Kim et al., 2015. Among these works of literature, one study about the lumbar facet joint osteoarthritis-induced spinal pain excluded the local inflammation or nerve injury (with negative straight leg raising test). Kim et al., 2015. These data also show the development of hind paw mechanical allodynia in the LSI model. One study demonstrated that the mouse sciatic nerve predominantly origins from the L3 and L4 DRG by injecting retrograde labeling in the hind paw. Rigaud et al., 2008. The presently disclosed retrograde tracing data demonstrated that L3 DRG is also the partial origin of sensory nerves in the endplates of L4/5 in LSI mice. In addition, the dorsal horn of spinal cord receives inputs from several segmental DRGs. Traub and Mendell, 1988; Kato et al., 2004. The major monosynaptic input for the dorsal horn neurons in L4 segment is from the L4-L6 DRGs, the dorsal horn neurons in L3 segment is from the L2-L5 DRGs. Pinto et al., 2008. These anatomical features might be the basis of the hind paw mechanical hypersensitivity in the LSI model.

### 1.5. Methods

### 1.5.1 Human Subjects

Human endplate samples were obtained from patients undergoing spinal fusion surgery in the Department of Spine Surgery at Xiangya Hospital (Changsha, China). Ethics committee approvals and patient consent were obtained before harvesting human tissue samples. Detailed information about the patients and groups is provided in Table 1.

### 1.5.2 Mice

C57BL/6J (WT) male mice were purchased from Charles River Laboratories (Wilmington, Mass.). The mice were anesthetized at 2 months of age with ketamine (Vetalar, Ketaset, Ketalar; 100 mg/kg, intraperitoneally) and xylazine (Rompun, Sedazine, AnaSed; 10 mg/kg, intraperitoneally). Then, the LSI model was created by resecting the L3-L5 spinous processes and the supraspinous and interspinous ligaments to induce instability of the lumbar spine. Bian, 2016: Ariga et al., 2001; Miyamoto et al., 1991. Sham operations were performed by only detachment of the posterior paravertebral muscles from L3-L5 on a separate group of mice. For the time-course experiments, mice were euthanized with an overdose of isoflurane (Forane, Baxter) inhalation at 2, 4, 8, or 12 weeks after LSI or sham surgery (10-12 per group). For the aging-induced endplate degeneration model, 3- and 20-month-old C57BL/6J (WT) male mice were purchased from Jackson Laboratory (10-12 per group).

The Avil-Cre mouse strain was provided by Dr. Xinzhong Dong (The Johns Hopkins School of Medicine, Baltimore, Md.). The EP4^{flox/flox} mouse strain was obtained from Dr. Brian L. Kelsall (National Institutes of Health, Bethesda, Md.). The Trap-Cre mouse strain was obtained from Dr. J. J. Windle (Virginia Commonwealth University, Richmond, Va.). The Netrin-1^{flox/flox} mouse strain was provided by Dr. H. K Eltzschig (University of Texas Health Science Center at Houston, Houston. Tex.). Dnp1-Cre and Rankl^{flox/flox} mouse strains were purchased from the Jackson Laboratory (Bar Harbor, Me.).

Heterozygous male Avil-Cre mice were crossed with EP4^{flox/flox} mice. The offspring were intercrossed to generate the following genotypes: WT, Avil-Cre (mice expressing (re recombinase driven by Advilin promoter). EP4^{flox/flox} (mice homozygous for the EP4 flox allele, referred to as FP4^{f/f} herein) and Avil-Cre, EP4^{flox/flox} (conditional deletion of EP4 in Advillin lineage cells, referred to as EP4^{-/-} herein). Heterozygous Dmp1-Cre mice were crossed with Rankl^{flox/flox}; the offspring were intercrossed to generate the following genotypes: WT, Dnp1-Cre (mice expressing Cre recombinase driven by Dmp1 promoter), Rankl^{flox/flox} (mice homozygous for the Rankl flox allele, referred to as Rankl^{f/f} herein), Dmp1-Cre; Rankl^{flox/flox} (conditional deletion of Rankl in DMP1⁺ lineage cells, referred to as Rankl^{-/-} herein) mice. Heterozygous Trap-Cre mice were crossed with Netrin-1^{flox/flox}; the offspring were intercrossed to generate the following genotypes: WT, Trap-Cre (mice expressing Cre recombinase driven by Trap promoter). Netrin-1^{flox/flox} (mice homozygous for the Netrin-1 flox allele, referred to as Netrin-1^{f/f} herein). Trap-Cre: Netrin-1^{flox/flox} (conditional deletion of Netrin-1 in TRAP⁺ lineage cells, referred to as Netrin-1^{-/-} herein) mice. The genotypes of the mice were determined by PCR analyses of genomic DNA, which was extracted from mouse tails within the following primers: Avil-Cre: Forward: 5′-CCCTGTTCACTGTGAGTAGG-3′ (SEQ ID NO: 1). Reverse: 5′-GCGATCCCTGAACATGTCCATC-3′(SEQ ID NO: 2). WT: 5′-AGTATCTGGTAGGTGCTTCCAG-3′(SEQ ID NO: 3): FP4 loxP allele Forward: 5′-TCTGTGAAGCGAGTCCTTAGGCT-3′(SEQ ID NO: 4). Reverse: 5′-CGCACTCTCTCTCTCCCAAGGAA-3′(SEQ ID NO: 5): Dmp1-Cre. Forward: 5′-CCCGCAGAACCTGAAGATG-3′(SEQ ID NO: 6), Reverse: 5′-GACCCGGCAAAACAGGTAG-3′(SEQ ID NO: 7), Rankl loxP allele: Forward: 5′-CTGGGAGCGCAGGTTAAATA-3′(SEQ ID NO: 8), Reverse: 5′-GCCAATAATTAAAATACTGCAGGAAA-3′(SEQ ID NO: 9); Trap-Cre: Forward: 5′-ATATCTCACGTACTGACGGTGGG-3′(SEQ ID NO: 10), Reverse: 5′-CTGTTTCACTATCCAGGTTACGG-3′(SEQ ID NO. 11); Netrin-1 loxP allele: Forward: 5′-AGGTAAAGTCTCCTACGCGG-3′(SEQ ID NO: 12). Reverse: 5′-CTTCCAAACCTGAACCGCCC-3′(SEQ ID NO: 13). LSI or sham surgery was performed in 2-month-old male EP4^{f/f}, EP4^{-/-}, Rankl^{-/-}, Rankl^{-/-}, Netrin-1^{f/f}, and Netrin-1^{-/-} mice. These mice were euthanized with an overdose of isoflurane inhalation at 4 or 8 weeks after LSI or sham surgery (12 per group) All mice were maintained at the animal facility of The Johns Hopkins University School of Medicine. All experimental protocols were approved by the Animal Care and Use Committee of The Johns Hopkins University, Baltimore. Md.

### 1.5.4 μCT

Mice were euthanized with an overdose of isoflurane inhalation and flushed with phosphate-buffered saline (PBS) for 5 min followed by 10% buffered formalin perfusion for 5 min via the left ventricle. Then, the lower thoracic and whole lumbar spine were dissected and fixed in 10% buffered formalin for 48 h, transferred into PBS, and examined by high-resolution sCT (Skyscan1172). The scanner was set at a voltage of 55 kVp, a current of 181 μA, and a resolution of 9.0 μm per pixel to measure the endplates and vertebrae. The ribs on the lower thoracic spine were included for identification of L4-L5 unit localization. Images were reconstructed and analyzed using NRecon v 1.6 and CTAn v 1.9 (Skyscan US, San Jose, Calif.), respectively. Coronal images of the L4-L5 unit were used to perform 3-dimensional histomorphometric analyses of the caudal endplate. Coronal images of the L5 vertebrae were used to perform 3-dimensional histomorphometric analyses of the trabecular bone or cortical bone (anterior shell). Three-dimensional structural parameters analyzed were total porosity and Tb.Sp for the endplates, trabecular BV/TV, Tb.N. Tb.Th, and Th. Sp for L5 vertebrae. Six consecutive coronal-oriented images were used for showing 3-dimensional reconstruction of the endplates and the vertebrae using 3-dimensional model visualization software. CTVol v2.0 (Skyscan US).

1.5.5 Histochemistry, Immunohistochemistry, and Histomorphometry

At the time of euthanasia, the L3-L5 lumbar spine and DRGs were collected and fixed in 10% buffered formalin for 48 h or overnight. Then, the spine samples were decalcified in 10% or 0.5M EDTA (pi 7.4) for 14 or 5 d and embedded in paraffin or optimal cutting temperature (OCT) compound (Sakura Finetek. Torrance. Calif.). Four-μm-thick coronal-oriented sections of the L4-L5 lumbar spine were processed for Safranin O and fast green, TRAP (Sigma-Aldrich), and immunohistochemistry staining using a standard protocol. Thirty-μm-thick coronal-oriented sections were prepared for sensory nerve- and blood vessel-related immunofluorescent staining, and ten-μm-thick coronal-oriented sections were used for other immunofluorescent staining using a standard protocol. The sections were incubated with primary antibodies to mouse endomucin (1:50, sc-65495. Santa Cruz Biotechnology), CD31 (1:50, ab28364, Abcam). CGRP (1:100, ab81887, Abcam), PGP9.5 (1:100, ab10404. Abcam), DCC (1:100, ab201260, Abcam), Netrin-1 (1:100, ab39370, Abcam), TRAP (1:200, ab185716, Abcam). Cox-2 (1:100, ab15191. Abcam), EP4 (1:10, ab92763, Abcam), IB4 (1:100, I21411. Thermo Fisher Scientific, Waltham, Mass.), Naᵥ 1.8 (1:100, ab93616, Abeam) PGE2 (1:100, ab2318, Abeam) overnight at 4° C. Then, the corresponding secondary antibodies were added onto the sections for 1 h while avoiding light. For immunohistochemistry, a horseradish peroxidase-streptavidin detection system (Dako) was subsequently used to detect the immunoactivity, followed by counterstaining with hematoxylin (Sigma-Aldrich). For immunofluorescent staining, the sections were counterstained with 4′,6-diamidino-2-phenylindole (DAPI, Vector, H-1200). The sample images were observed and captured by a fluorescence microscope (Olympus BX51, DP71) or confocal microscope (Zeiss LSM 780). ImageJ (NIH) software was used for quantitative analysis. Endplate scores were calculated as described previously. Boos et al., 2002; Masuda et al., 2005.

### 1.5.6 Retrograde and Anterograde Tracing

2-month-old male C57BL/6J mice (Charles River) were used to perform LSI or sham surgery (6 per group). The mice were anesthetized with ketamine and xylazine at 8 weeks after surgery. For the retrograde nerve tracing experiment, the caudal endplate of L4-L5 was adequately exposed with a ventral approach. Then, 2 μL Dil (Molecular Probes; 2 mg/mL in methanol) was injected into the left part of caudal endplate of L4-L5 using borosilicate glass capillaries after drilling a hole at left part of endplate. The drilling holes were sealed with bone wax immediately after injection to prevent tracer leakage. After Dil injection, the wound was sutured, and a heating pad was used to protect mice during recovery from anesthesia Mice were euthanized with an overdose of isoflurane inhalation, and the DRGs (T12-L6) were isolated for immunofluorescence at 1 week after Dil injection. Ten-μm-thick frozen sections were used, and the Dil signals were inspected under 564-nm excitation using a confocal microscope (LSM 780, Zeiss).

For the anterograde tracing experiment, 2-month-old male C57BL/6J mice (Charles River) were used to perform LSI or sham surgery and 3- and 20-month-old male C57BL/6J mice (Jackson Laboratory) were prepared for the aging-induced endplate degeneration model (6 per group). The aging model mice and the operated mice were anesthetized at 8 weeks after surgery with ketamine and xylazine. The L1 and L2 DRGs were adequately exposed with a dorsal approach. Then, 2 μL Dil (Molecular Probes; 2 mg/mL in methanol) was injected into the DRGs using borosilicate glass capillaries. After Dil injection, the wound was sutured, and a heating pad was used to protect mice during recovery from anesthesia. Mice were euthanized with an overdose of isoflurane inhalation, and the L3-L5 spine was collected for immunofluorescence at 1 week after Dil injection. Thirty-μm-thick coronal-oriented frozen sections were used, and the Dil signals were inspected under 564-nm excitation using a confocal microscope (LSM 780, Zeiss).

1.5.7 qRT-PCR

Total RNA was extracted from lumbar spinal endplate tissue samples using TRIzol reagent (Invitrogen. Carlsbad, Calif.) according to the manufacturer's instructions. The purity of RNA was tested by measuring the ratio of absorbance at 260 nm over 280 nm. For qRT-PCR, 1 μg RNA was reverse transcribed into complementary DNA using the SuperScript First-Strand Synthesis System (Invitrogen), then qRT-PCR was performed with SYBR Green-Master Mix (Qiagen, Hilden, Germany) on a C1000 Thermal Cycler (Bio-Rad Laboratories, Hercules, Calif.). Relative expression was calculated for each gene by the 2⁻^{_{ΔΔ}}^{CT} method, with glyceraldehyde 3-phosphate dehydrogenase for normalization. Primers used for qRT-PCR are listed in Table 2.

TABLE 2
The primers sequence for qRT-PCR

| | | SEQ | | SEQ |
|---|---|---|---|---|
| Target | Forward primer | ID | Reverse primer | ID |
| gene | (5′-3′) | NO. | (5′-3′) | NO. |
| PGE | TTTCTGCTCTGCAG | 14 | GATTGTCTCCATGT | 21 |
| synthase | CACACT | | CGTTGC | |
| (PGES) | | | | |
| cox-2 | CAGACAACATAAAC | 15 | GATACACCTCTCCA | 22 |
| | TGCGCCTT | | CCAATGACC | |
| IL-1β | TTCAGGCAGGCAGT | 16 | CGTCACACACCAGC | 23 |
| | ATCACTC | | AGGTTAT | |
| IL-17 | TCTCCACCCCAATG | 17 | CACACCCACCAGCA | 24 |
| | AAGACC | | TCTTCT | |
| IL-2 | TTGTGCTCCTTGTC | 18 | CTGGGGAGTTTCAG | 25 |
| | AACAGC | | GTTCCT | |
| TNF-α | ALGAGCACAGAAAG | 19 | AGTAGACAGAAGAG | 26 |
| | CAPG | | CGTGGT | |
| GAPDH | AATGTGCCGTCGTG | 20 | AGTGTAGCCCAAGA | 27 |
| | GATCTGA | | TGCCCTTC | |

### 1.5.8 ELISA

The concentrations of PGE2 and netrin-1 in the L3-L5 endplates were determined by using the PGE2 Parameter Assay Kit (KGE004B, R&D Systems) and Mouse Netrin-1 ELISA Kit (EKC37454, Biomatik, Wilmington, Del.) according to the manufacturer's instructions (3 per group), respectively.

### 1.5.9 DRG Neuron Culture

DRG neuron culture was processed as described previously. Saijilafu and Zhou, 2012. Briefly, the dishes or coverslips for DRG neuron culture were coated with 50 μL working solution containing 100 μg/mL Poly-D-Lysine and 10 g/mL Laminin at 37° C. To prepare the neuron culture medium, alpha minimum essential medium (α-MEM) was supplemented with 1× penicillin-streptomycin solution (500 units of penicillin and 500 μg of streptomycin, Gibco Laboratories, Gaithersburg, Md.), 5% fetal bovine serum (Gibco), 1× GlutaMAX-1 supplement (35050-061, Thermo Fisher Scientific), and the antimitotic reagents containing 20 μM 5-fluoro-2-deoxyuridine (F0503, Sigma-Aldrich) and 20 μM uridine (U3003, Sigma-Aldrich). For the serum-free medium, the fetal bovine serum was replaced with the supplement B27. After euthanizing the 6- to 8-week-old mice, the lumbar DRGs were harvested and stored in microfuge tubes with α-MEM medium placed on ice. DRG neurons were digested and dissociated with 1 mg/mL collagenase A solution (10103578001, Roche, Basel, Switzerland) in a 37° C. incubator for 90 min followed by 1× TrypLE Express solution (15140-122, Thermo Fisher) at 37° C. for 20 min. Then, TrypLE Express solution was removed, and DRGs were washed gently 3 times with 1 mL prepared culture medium (containing 5% fetal bovine serum). Tissue was triturated by gently pipetting up and down 20-30 times in 1 mL prepared culture medium. After trituration, the DRG neuron suspension was filtered (40-μm strainer) following non-dissociated tissue settlement to the bottom of the microfuge and transferred to another tube. After centrifugation (800 r.p.m for 4 min at room temperature), the cell pellet was resuspended and cultured in a precoated dish.

### 1.5.10 Western Blot

The primary DRG neurons were treated with 20 μM PGE2(14010, Cayman Chemical, Ann Arbor, Mich.) for 30 min; 10 μM PKA inhibitor (H-89, BML-E1196, Enzo Life Sciences, Farmingdale, N.Y.) for 1 h. Western blot analysis was conducted on the protein lysates from the cultured primary DRG neurons. The supernatants of lysates were collected after centrifugation and separated by SDS-PAGE (sodium dodecyl sulfate-polyacrylamide gel electrophoresis) and then blotted on the Nitrocellulose Blotting Membranes (Bio-Rad Laboratories). Specific antibodies were applied for incubation, and the proteins were detected by using an enhanced chemiluminescence kit (Amersham Bioscience, RNP2108). The antibodies used for western blot were pCREB (1:1000, 9198, Cell Signaling Technology), CREB (1:000, 9197, Cell Signaling Technology), pPKA (1:1000, 5661, Cell Signaling Technology), PKA (1:1000, 4782S. Cell Signaling Technology), and p-tubulin (1:2000, MA5-16308, Invitrogen). The original blots are provided in the Source Data file.

### 1.5.11 Sodium Indication

For sodium indication. ANG-2 AM (Teflabs, Austin, Tex.) was used according to the manufacturer's protocol. Briefly, 1 mM stock solution of ANG-2 AM was diluted to twice the original volume with a solution of 20% Pluronic F-127 (Thermo Fisher Scientific) in DMSO. Then, the ANG-2 AM/Pluronic F-127 solution was dispersed to final concentration at 5 μM ANG-2 AM and 0.1% Pluronic F-127 with serum-free culture medium. After incubation for 1 h at 37° C., the cell loading medium was removed. The cells were washed with serum-free and dye-free medium and prepared for sodium imaging. The sterile imaging buffer contained 5.4 mM KCL, 160 mM NaCL, 20 mM HEPES, 1.3 mM CaCL₂, 0.8 mM MgSO₄, 0.78 mM NaH₂PO₄, and 5 mM glucose (pH 7.4) The sodium imaging was monitored and captured using a confocal microscope (Zeiss LSM 780).

In different sets of experiments, the DRG neurons were treated with 20 μM PGE2 (14010, Cayman Chemical, Ann Arbor, Mich.) for 5 min. 10 μM PKA inhibitor (H-89, BML-E1196, Enzo Life Sciences, Farmingdale, N.Y.) for 1 h, dibutyryl-cAMP (28945. Sigma-Aldrich) for 5 min, or siRNA oligo for Naᵥ 1.8 (GE Healthcare Dharmacon, Lafayette, Colo.). The siRNA transfection was by using Lipofectamine RNAiMAX Transfection Reagent (Thermo Fisher Scientific) using a standard protocol.

For immunostaining, the DRG neurons were washed 3 times with PBS, followed by fixation by 4% paraformaldehyde (PFA) for 20 min at room temperature. The immunofluorescent staining used a standard protocol. The coverslips were incubated with primary antibodies to mouse CREB (1.100, 9197. Cell Signaling Technology), p-CREB (1:100, ab32096, Abcam), CGRP (1:100, ab81887, Abeam), PKA (1:100, 4782, Cell Signaling Technology), and p-PKA (1:100, ab227848, Abcam) overnight at 4° C. Then, the corresponding secondary antibodies were added onto the coverslips for 1 h while avoiding light. The coverslips were counterstained with 4′,6-diamidino-2-phenylindole (DAPI, Vector, H-1200). The sample images were observed and captured using a microscope (Olympus BX51, DP71).

### 1.5.12 Behavioral Testing

Behavioral testing was performed before surgery and weekly after surgery. All behavioral tests were performed by the same investigator, who was blinded to the study groups. Vocalization thresholds in response to the force of an applied force gauge (SMALGO algometer: Bioseb. Pinellas Park, Fla.) were measured as pressure hyperalgesia. Yu et al., 2012.

A 5-mm-diameter sensor tip was directly pressed on the dorsal skin over L4-L5 (0.5 cm above the line connecting posterior iliac crest) while the mice were gently restrained. The pressure force was increased at 50 g/sec until an audible vocalization was made. The curve of pressure force was recorded by using BIO-CIS software (Bioseb) to ensure the force increased gradually. A cutoff force of 500 g was used to prevent tissue trauma. Two tests were performed 15 min apart, and the mean value was calculated as the nociceptive threshold.

Spontaneous wheel-running activity was recorded using activity wheels designed for mice (model BIO-ACTIVW-M, Bioseb). Cobos et al., 2012. The software enabled recording of activity in a cage similar to the mice's home cage, with dimensions of 35×20×13 cm, and the wheel (diameter: 23 cm, lane width: 5 cm) could be spun in both directions. The device was connected to an analyzer that automatically records the spontaneous activity. The mice had ad libitum access to food and water. The distance traveled, mean speed, maximum speed, and total active time during 2 days were evaluated for each mouse.

The hind paw withdrawal frequency in response to a mechanical stimulus was determined using von Frey filaments of 0.7 mN and 3.9 mN (Stoelting, Wood Dale, Ill.). Mice were placed on a wire metal mesh grid covered with a clear plastic cage. Mice were allowed to acclimatize to the environment for 30 min before testing Von Frey filaments were applied to the mid-plantar surface of the hind paw through the mesh floor with enough pressure to buckle the filaments. Probing was performed only when the mouse's paw was in contact with the floor. A trial consisted of application of a von Frey filament to the hind paw 10 times at 1-sec intervals. If withdraw occurred after application, it was recorded, and the next application was performed similarly when the mouse's paw was again in contact with the floor. Mechanical withdrawal frequency was calculated as the percentage of withdrawal times in response to 10 applications.

Straight leg raising test was performed by stretching the hindlimb (knee joint fully extended) and flexing the hip for 2 seconds. The number of vocalizations in 5 leg stretch-and-lifts were recorded. Kim et al., 2015: Kroin et al., 2005. The negative result indicates that the nerve root compression is not involved in the hyperalgesia developed after LSI surgery.

### 1.5.13 Statistics

All data analyses were performed using SPSS, version 15.0, software (IBM Corp.). Data are presented as means±standard deviations. For comparisons between two groups, unpaired, two-tailed Student's t-tests were used. For comparisons among multiple groups, one-way ANOVA with Bonferroni's post hoc test was used. For all experiments. P<0.05 was considered to be significant. All inclusion/exclusion criteria were preestablished, and no samples or animals were excluded from the analysis. No statistical method was used to predetermine the sample size. The experiments were randomized, and the investigators were blinded to allocation during experiments and outcome assessment. The same sample was not measured repeatedly.

### Example 2

### Parathyroid Hormone Attenuates Low Back Pain by Reducing Sensory Innervation in Porous Endplates

### 2.1 Background

Low back pain (LBP) is a common clinical and public health problem. LBP profoundly affects quality of life and daily physical activity, especially in the elderly population, increasing risk of frailty and loss of socioeconomic function. It is estimated that disability-adjusted life-years (DALYs) caused by low back pain has risen from 17th in 1990 to 13th in 2017 in China among all diseases. Zhou et al., 2019, and was the first leading cause of years lived with disability (YLD) worldwide in 2016. Global, regional, and national incidence, prevalence, and years lived with disability for 328 diseases and injuries for 195 countries, 1990-2016: a systematic analysis for the Global Burden of Disease Study 2016. With an aging population and developing society, the socioeconomic burden is enormous. An estimated 149 million work days are lost every year in the United States alone because of LBP, Manchikanti, 2000, with total costs estimated to be $100-200 billion annually. Katz, 2006.

Several pathologies, like lumbar disc hemiation (LDH), scoliosis, tumor and infection, can result in low back pain. The gold standard therapy remains orthopedic surgical intervention, including: disc arthroplasty, decompression, artificial disc replacement, and spinal fusion. Wenger and Cifu, 2017; Hartvigsen et al., 2018. However, non-specific LBP (i.e., without known pathoanatomical cause) accounts for about 90% of total LBP cases, Koes et al., 2006, including the asymptomatic early-stage disc degenerative disease (DDD).

Despite the high prevalence of non-specific LBP, no ideal intervention is available. Instead, education and reassurance, pharmacological and non-pharmacological therapies have been adopted in various guidelines, but none of these therapies are targeted. In terms of pharmaceutical efficacy and WHO analgesic ladder, the four major classes of medications indicated for acute LBP are paracetamol, opioids, non-steroidal anti-inflammatory drugs (NSAIDs, including COX-2 inhibitors), and skeletal muscle relaxants, while tricyclic antidepressants and benzodiazepines are recommended for chronic cases. Maher et al., 2017.

Other minor classes, such as triptans, 2-agonists, and local anesthetics, have more specific, but limited effects on certain types of pain (i.e., mostly acute) or related disorders. These medications are accompanied with side effects ranging from addiction, respiratory depression, constipation (opioids), gastrointestinal and cardiovascular damage (NSAIDs), to a myriad of negative central nervous system (CNS) effects (anticonvulsants and antidepressants). Additionally, LBP is prone to recurrence after withdrawal. In all of these circumstances, it is believed that patients need not pain killers, but disease modifying therapies that abrogate or abolish pain. Due to the limited understanding of non-specific LBP pathogenesis during early- and middle-staged DDD, however, there is no disease modifying drugs (DMDs) to prevent and treat it.

Most evidence presented thus far attributes low back pain to DDD in the lumbar region. Luoma et al., 2000. This evidence suggests its etiology is multifactorial and affected by genetics, Battie et al., 2008; gender, Miller et al., 1988; aging, Powell et al., 1986; high mechanical stress, Videman et al., 1995; and disc dystrophy, Benneker et al., 2005. Gullbrand et al., 2015. The anatomically intact intervertebral disc (IVD), especially the nucleus pulposus (NP), is commonly considered an avascular organ except for the outer annulus fibrosus (AF) layer. Nutrients reach the IVD predominantly by diffusion through the vertebral endplate, so the NP has precarious nutrition. Rodriguez et al., 2012. Endplate has been demonstrated to be remodeled during aging, undergoing gradual ossification with elevated osteoclasts number and increased porosity, Rodriguez et al., 2012, Jia et al., 2016; Bian et al, 2016; Bian et al., 2017; and Papdakis et al., 2011, thereby leading to disc degeneration. Gruber et al., 2005; Gruber et al., 2007. In previous studies. Wei et al, 2015; Zhou et al., 2013, an intervertebral disc degeneration model was established in rhesus monkeys by injecting pingyangmycin, an anti-endothelial drug, into the sub-endplate region to block the blood sinus in the endplate. These studies supported the dystrophy disorder hypothesis.

Moreover, the endplate degeneration also showed a high correlation with LBP. Jensen et al., 2008. Patients with osteoporosis are usually affected with discogenic LBP, so this phenomenon was reproduced in an OVX-induced osteoporosis rhesus monkey model. The disc degeneration in the OVX group was more severe than that of the control. Zhong et al., 2016. Endplate remodeling also was confirmed by increased calcification and porosity, as well as decreased vascularization in the endplates adjacent to the degenerated IVDs. These pathological features exacerbated degeneration of the IVDs in the animals with osteoporosis. The data suggested that the endplate becomes sclerotic from elevated remodeling of the cartilage under normal physiological weight bearing during aging. In addition, similar endplate changes were observed under aberrant mechanical loading in the lumbar spine instability (LSI) mice model. Bian, 2016. Endplates become sclerotic after LSI because high levels of TGF-β are activated by osteoclast-mediated resorption of the cartilage matrix. A more recent study revealed that sensory innervation accompanies the osteoclast-mediated initiation of endplate sclerosis, resulting in LBP (unpublished results). Without wishing to be bound to any one particular theory, it is thought that LBP can be attenuated by preventing endplate remodeling and sclerosis, ultimately slowing disc degeneration.

Parathyroid hormone (PTH), a systemic hormone that regulates calcium homeostasis, plays a major role in orchestrating bone remodeling by modulating the bone marrow microenvironment and the signaling of local factors. Bikle et al., 2002; Canalis et al., 1989: Miyakoshi et al., 2001: Pfeilschifter et al., 1995: Wein and Kronenberg, 2018: Fan et al., 2017: Wan et al., 2008; Yu et al., 2012; and Qui et al., 2010.

Teriparatide (PTH(1-34′)) is an analogue of PTH and consists of the first (N-terminus) 34 amino acids. It is an FDA-approved anabolic agent to stimulate bone formation for treatment of osteoporosis. PTH(1-34′) not only improved bone density in patients with osteoporosis, but also attenuated LBP in various cases. For example, PTH ameliorated LBP for at least 18 months after discontinuation. Fahrleitner-Pammer et al., 2011: Jakob et al., 2012, which suggests the pain relief resulted from structural remodeling of the spine components, apart from the improvement of osteoporotic conditions. Although several clinical studies have reported similar phenomena, Fahrleitner-Pammer et al., 2011; Langdahl et al., 2016: and Koski et al., 2013, no biological mechanism has been described.

It also has been recently reported that the cilia of NP cells mediates mechanotransduction to maintain anabolic activity in the IVD. Zheng et al., 2018. It was found that mechanical stress promotes transport of the type 1 parathyroid hormone receptor (PTH1R) to the cilia and enhances PTH signaling in NP cells. Daily injection of PTH effectively attenuated disc degeneration of aged mice by direct signaling through NP cells. Given sensory innervation in the pores of endplate is associated with LBP, daily injection of PTH can increase bone formation and attenuate disc degeneration in animals and provide relief of back pain. In the present example, the role of sensory innervation in vertebral endplates and the remodeling of sclerotic endplates in the attenuation of LBP after daily injection of PTH was investigated.

### 2.2 Results

### 2.2.1 PTH Attenuates Low Back Pain in Mice

To examine the potential effect of PTH on spinal degeneration and pain, lumbar spine instability (LSI) and aging mouse models were established. LSI or sham mice were injected with PTH and hyperalgesia tests of pressure tolerance were performed for their low back pain (FIG. 2-1A). The value of pressure tolerance significantly decreased 8 weeks post LSI surgery relative to sham surgery (FIG. 2-1B), indicating development of low back pressure hyperalgesia. Interestingly, this low back pressure hyperalgesia was significantly attenuated with daily injection of PTH in LSI mice in comparison with vehicle (FIG. 2-1B). Spontaneous activity was also measured by active wheel. The results showed that mouse active time per 24 hours, active time, distance traveled, and mean speed of movement decreased significantly 8 weeks post LSI surgery relative to sham mice. Again, PTH treatment significantly increased the spontaneous activity (FIG. 2-1C-FIG. 2-E). In parallel, PTH effect on spine pain of aged mice also was evaluated (FIG. 2-1F). Similarly, PTH significantly increased both the threshold of pressure tolerance (FIG. 2-1G) and spontaneous activity (FIG. 2-1H-FIG. 2-1J) in 18-month-old mice. Collectively, these results suggest that PTH could attenuate LBP during spine degeneration.

### 2.2.2 PTH Increased the IVD Space by Decreasing the Volume and Porosity of Sclerotic Endplates

Whether PTH-reduced LBP modified spine degeneration also was examined. Previous studies have demonstrated that sclerosis of endplates decreases IVD space and increased porosity and volume of the endplate in both LSI and aging mice. Indeed, endplates in 4-month sham-operation mice showed few cavities detected in μCT images (FIG. 2-2A), which was further confirmed with safranin O/fast green staining with red color indicating normal cartilage structures (FIG. 2-2B). Endplates in LSI or aged mice showed increased porosity and volume (FIG. 2-2C-FIG. 2-2E) in μCT images and significant loss cartilage matrix in safranin O/fast green staining. The green-stained bone matrix around the cavities indicates ossification. Importantly. PTH treatment significantly decreased the porosity and volume of endplates (FIG. 2-2C-FIG. 2-2E). PTH also significantly increased cartilage in LSI and aging mice quantitative analysis of safranin O/fast green staining (FIG. 2-2F). In addition, the height and volume of IVD and endplate was measured (FIG. 2-2G). Unexpectedly. PTH increased the height and volume of IVD with a decrease of endplate height in both LSI and aged mice (FIG. 2-2H). The observation indicates that PTH-reduced LBP is likely by improvement of spine degeneration.

### 2.2.3 Sensory Innervation Decreased in PTH Remodeling of Sclerotic Endplates

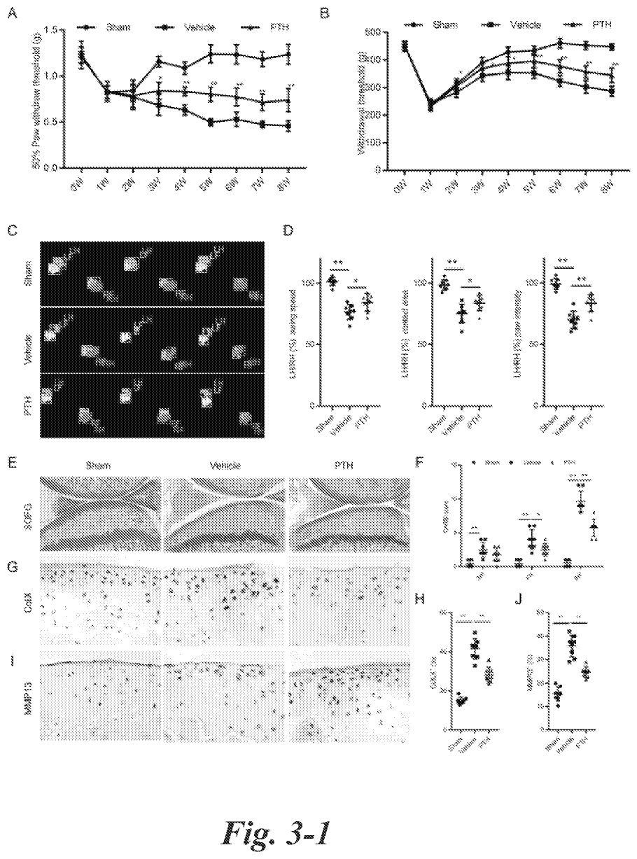

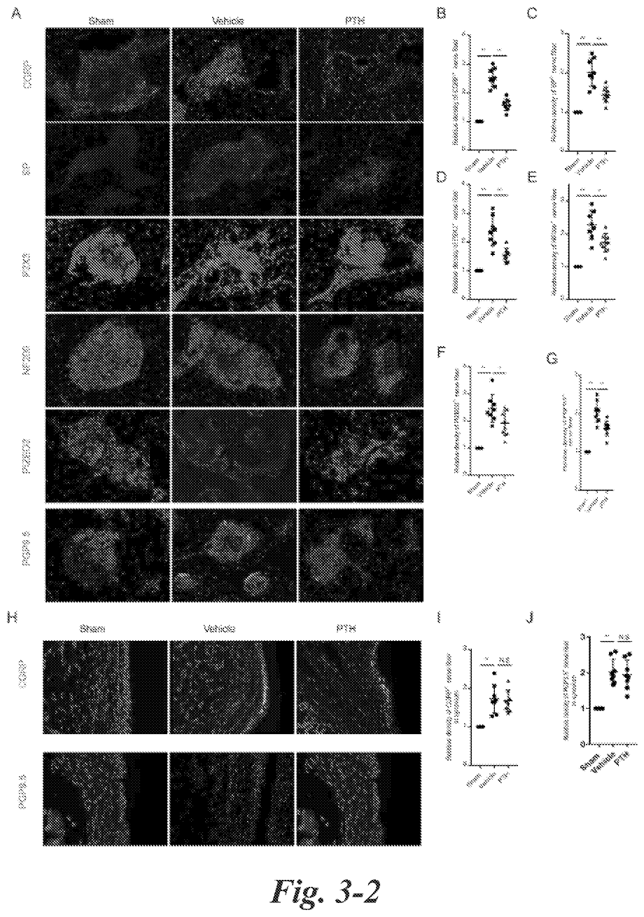

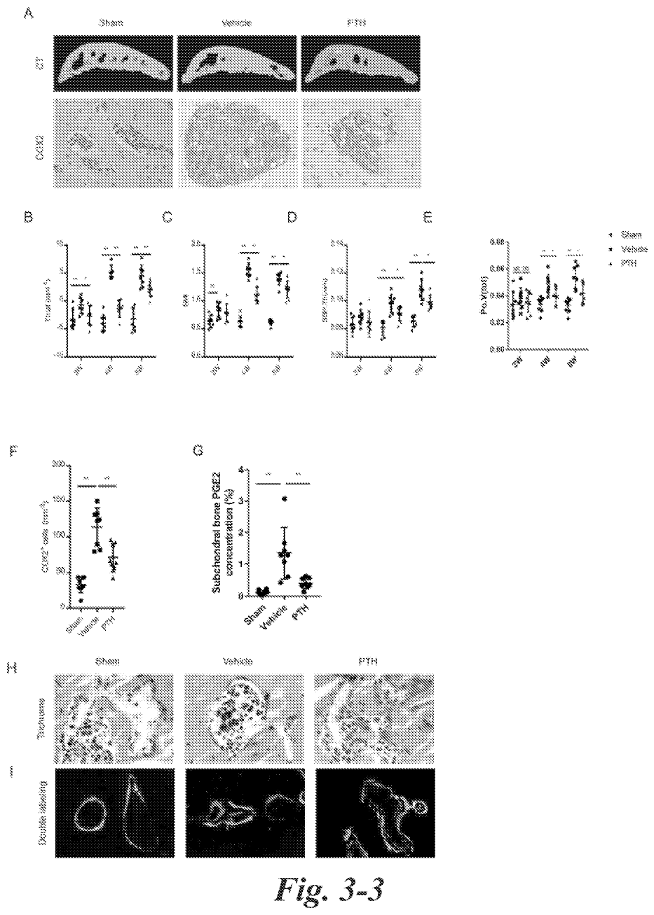

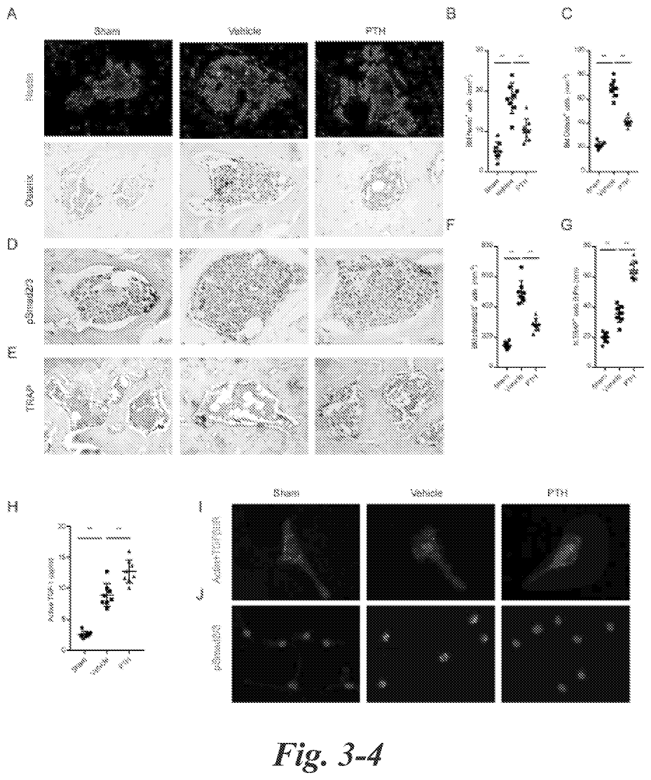

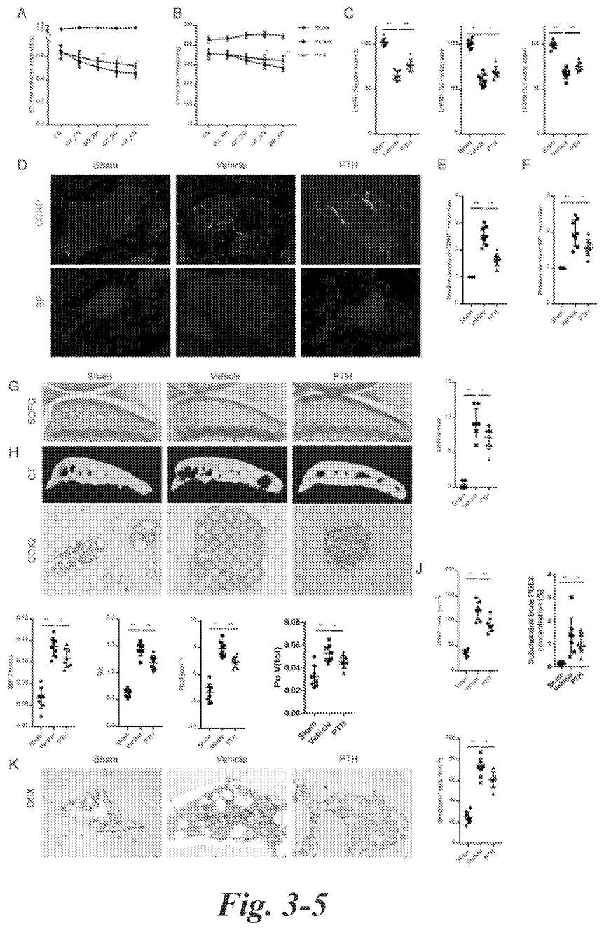

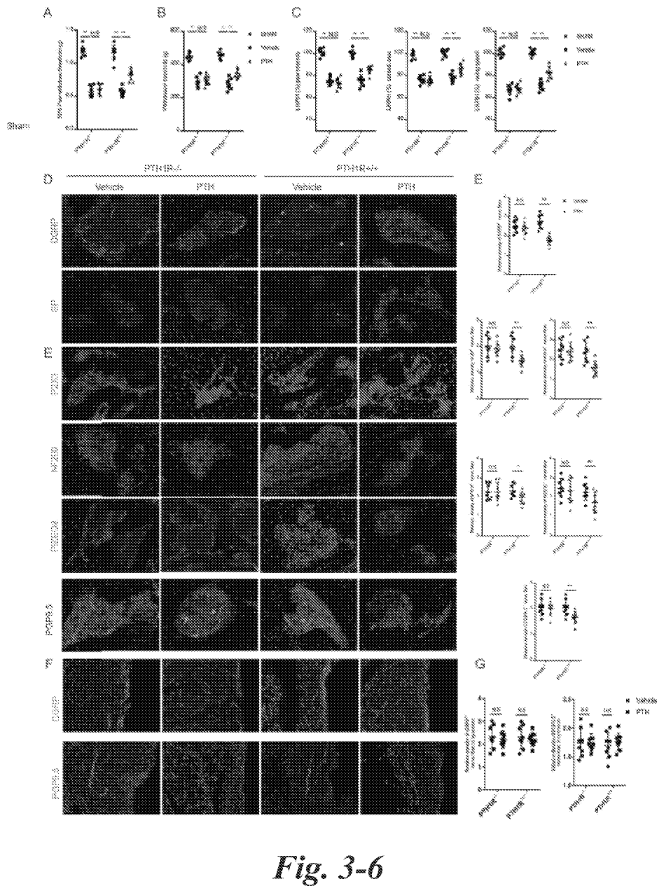

As previously reported, innervation of nerve fiber in the porous endplates or the inner 2/3 of annulus fibrosus were the sources of LBP. The change of sensory innervation in the sclerotic endplates and annulus fibrosus after iPTH was then examined. PGP9.5 is a broad marker of nerve fibers. Immunostaining of intervertebral disc sections with PGP9.5 showed that there is no PGP9.5 positive fibers in the sham endplates except for the periphery of annulus fibrosus (FIG. 2-3A), but it significantly increased in endplate of LSI and aged mice relative to sham-operated mice (FIG. 2-3B). Importantly. PGP9.5 positive nerve fibers were significantly reduced in the endplates of LSI and aged mice with iPTH treatment relative to vehicle (FIG. 2-3C). Although they were less increased in the annulus fibrosus after LSI and aged mice relative to sham-operated mice, there were no statistical differences in neither the vehicle group nor PTH treatment (FIG. 2-3D). This observation indicates that innervation of nerve fiber in the porous endplates, but not annulus fibrosus, were the primary source of LBP and affected by PTH.

To determine which kinds of nerve fibers grow into the endplate, a retrograde of DIL was performed by injecting it into L4-5 endplate as previously reported. L1-L2 DRG sections were immuno-stained with antibodies against PGP9.5, CGRP, IB4, P2X3, PIEZO2 or NF200 respectively for different sensory nerve fibers. The results showed that there were no DIL positive neuron in sham-operated mice (FIG. 2-3E). DIL positive neuron appeared in LSI and aging mice, but were significantly decreased in PTH treatment groups. In addition. DIL⁺ neurons were stained with CGRP, NF2100, especially in PIZO2, but not P2X3 or IB4, which means the nerve fiber from CGRP. NF200 and PIZO2 positive neuron grows into endplates (FIG. 2-3G). These markers also were immune-stained in endplate (FIG. 2-3E). The results also confirmed the phenomenon (FIG. 2-3F). It suggested innervation porosity endplates of peptidergic nociceptive C nerve fibers and especially mechanical pain related nerve fiber may be the source of pain and PTH may reduce the pain by reduction of sensory nerve fibers.

2.2.4 PTH-Induced Remodeling of Sclerotic Endplates Reduces their Porosity

To understand the potential mechanism of PTH treatment in increase of IVD space and reducing sensory innervation, whether PTH induces remodeling of sclerotic endplates was examined. Trap staining and osteocalcin immunostaining revealed that osteoclasts and osteoblasts were not observed in the endplates of sham operated mice, but significantly increased in the endplates post LSI surgery and aged mice (FIG. 2-4A). Particularly, the osteoclasts were primarily localized within the cavities of cartilage resorption in the vehicle-treated mice. Importantly, the number of osteocalcin-positive osteoblasts significantly increased in the cavities of endplates (FIG. 2-4B, FIG. 2-4G), but there were no changes of trap-positive osteoclasts in PTH-treated LSI aged mice (FIG. 2-4F), and the size of cavities were significantly smaller relative to the vehicle group. Moreover, CD31⁺EMCN⁺ immunofluorescence staining demonstrated that the type H blood vessels, which is specifically associated with bone formation, were barely detectable in the endplates of sham operated mice. LSI surgery and aged mice (FIG. 2-4C). However, PTH significantly increased the numbers of CD31⁺EMCN⁺ type H blood vessels in the endplates in both LSI mice and Aging mice relative to vehicle group, indicating PTH-induced anabolic bone formation in the cavities (FIG. 2-4H), which was validated by double labeling experiment (FIG. 2-4D). Golder staining further demonstrated that PTH significantly increased osteoid formation at the surface of cavities in the LSI and aged mice as compared with the vehicle-treated mice (FIG. 2-3F), which is consistent with an increase of osteoblasts in the cavities.

As the sensory nerve fibers were detected in the cavities, axon attractive factor Netrin-1, NGF played an important role in sensory innervation in the endplates. To examine if axon attractive and repulsive factors play a role in the reduction of sensory nerve fibers after iPTH, the mRNA expression of the potential factor by qPCR was measured. The results showed that iPTH stimulated expression of Slit-3 and Sema-3a in the endplates of LSI, but reduced the expression of NGF and inflammatory factors including PGE2 related genes although there was no significant change in netrin-1 in the bone formation. The observation suggested iPTH-induced decrease of porosity and increase of secretion of axon repulsive factors likely results in reduction of sensory innervation in the endplates.

### 2.25 IPTH Decreased Cox-2 Expression and PGE2 Levels by Reducing Porosity of Sclerotic Endplates

It has been previously shown that PGE2 secreted by osteoblastic cells activates PGE2 receptor 4 (EP4) in sensory nerves to maintain bone homeostasis by modulation of sympathetic activity through the central nervous system (Chen et al., 2019) and PGE2 levels in the bone are positively correlated with bone density. Porous endplates resemble osteoporotic bone elevated PGE2 levels, which stimulate sensory nerves that lead to LBP (in press). Thus, whether porosity structure of endplates enhances mechanical loading-stimulated PGE2 secretion by finite element analysis was examined (FIG. 2-5A). The results showed that high-stress concentration occurred in the large cavities area of sclerotic endplates of LSI and aging mice relative to sham-operated mice, in which there was no high-stress concentration occurred in the whole endplates. Stress concentrations were significantly released in iPTH-treated LSI and aged mice compared to vehicle group as porous endplates were remolded to smaller cavities (FIG. 2-5C). Therefore, whether PGE2 protein levels also changed in the endplates with iPTH treatment was examined. The results showed that elevated PGE2 levels and COX-2 expression were observed in ELISA (FIG. 2-5F) and immunostaining in the sclerotic endplates of LSI and aged mice relative to sham-operated mice (FIG. 2-5B). iPTH significantly reduced COX-2 expression and PGE2 levels in the sclerotic endplates of LSI and aged mice relative to vehicle group (FIG. 2-5D, FIG. 2-5E). Thus, iPTH-induced remodeling of sclerotic endplates reduces PGE2 levels for LBP.

### 2.2.6. Knock Out PTH Receptor in Osteoblast Cell but not Nucleus Pulposus Cell Blunted the Effect of PTH Attenuating Spine Pain

PTH binds to the type 1 parathyroid hormone receptor (PTH1R), which is expressed in high levels in osteoblast and osteocyte cells in bone and regulates bone homeostasis through activation of adenylate cyclase and phospholipase C. Deletion of PTH1R ensures blocking of the PTH signaling cascade. Knockout of PTH1R in osteocalcin-positive osteoblast cells of LSI mice was induced to confirm the nerve repulsion role of PTH at the remolding of porosity endplate. Similarly to wild type mice with PTH treatment, the total porosity and endplate volume were significantly reduced in the PTHR f/f mice after 2 months iPTH treatment post-surgery relative to the vehicle treatment.

As reported in a previous study, daily injection of PTH effectively attenuated disc degeneration of aged mice with expressed aggrecan by direct signaling through NP cells, which cannot only induce DRG growth cone collapse, but also inhibit DRG axonal growth. Zheng, L., et al., 2018. Knockout of PTH1R was induced in notochord cells of LSI mice to confirm if the nerve repulsion factors from nucleus pulposus after PTH treatment.

2.27 iPTH Attenuates Endplate Sclerosis and Disc Degeneration in Aging Monkey

To confirm the effects of iPTH in the disc degeneration, especially the endplate sclerosis, eight aging monkeys, which had similar disc degenerated grade in L1-L5 lumbar, were screened and half of them were treated for 3 months with iPTH, the others were treated with vehicle (FIG. 2-6). The results of the MRI showed that the signals of disc were increased after iPTH 3 m comparing to pre-treatment, while the signals were slightly decreased relative to 0 m in the vehicle group (FIG. 2-6A) And pfirrmann grade of these discs had been evaluated (FIG. 2-6E), the results also showed iPTH resulted in lower pfirrmann grade than pre-treatment. At the same time, there were decreased but no significant change within 3 m in the vehicle group (FIG. 2-6F). Moreover, Quantitative analysis of T1ρ and T2 map value also exhibited similar results, suggesting the disc degeneration had been attenuated by iPTH (FIG. 2-6B. FIG. 2-6C).

In addition, the signals change of disc after 3 months post-iPTH-treated-3 m also was monitored. Although the high signals of disc at iPTH 3 m had been reduced when stopping the injection of iPTH for 3 m, the signals of disc at vehicle group at 6 m had become lower as compared that at 0 m and 3 m. Further, the results of pfirrmann grade of vehicle 6 m were significantly increased than that at 0 m. Meanwhile, the decreased values of T1ρ and T2 map within 6 m in vehicle group also confirmed disc degeneration with aging, while the T1ρ and T2 map values increased after iPTH treatment, especially the T2 map values continued high expression in PTH group at 6 m, stopping the injection of iPTH for 3 m, indicated the function of disc regeneration after iPTH.

In summary, the endplate as the main structure contributing the nutrient diffusion from vertebrate to disc, which had been evaluated by micro-CT and tissue staining. As found in the above description of mice, large bone marrow cavities occurred in front of the endplate in aging monkey, but the porosity was significantly reduced in the iPTH-treated group. Moreover, the area of cartilage in the endplate also showed that they were significantly increased after iPTH. Thus, these data further demonstrated that iPTH can reduce sensory innervation and spine pain by remodeling of the porous endplate.

### Example 3

### Administration of Parathyroid Hormone Attenuates Knee Osteoarthritic Pam by Remodeling of Subchondral Bone

### 3.1 Overview

Osteoarthritis (OA) is a debilitating and leading prevalent joint degeneration characterized by joint pain and disability. The current treatment of OA fails to attenuate OA progression and decrease joint pain effectively. Here it is shown that PTH attenuates OA pain by inhibition of nerve innervation and subchondral bone deterioration and articular cartilage degeneration in DMM mouse model. It was found that sensory nerve innervation for OA pain is significantly decreased in PTH-treated DMM mice compared with vehicle-treated DMM mice. In parallel, deteriorated subchondral bone microarchitecture defined as higher trabecular pattern factor (Tb.pf), structure model index (SMI), and increased thickness of subchondral bone plate (SBP.Th) in vehicle-treated DMM control is attenuated by PTH treatment. PGE₂ in subchondral bone is increased in response to large porosity of subchondral bone in DMM mice. Furthermore, uncoupled subchondral bone remodeling by increased TGFβ signaling is regulated by PTH-induced endocytosis of the PTH1R-TβRII complex. Notably, conditional knockout the PTH type I receptor in nestin⁺ MSCs eliminates PTH-induced improved deteriorated subchondral bone microarchitecture, subsequent and PGE₂ concentration in the subchondral bone and decreased sensory nerve innervation for OA pain. These studies reveal that PTH attenuates OA progression and OA pain by inhibition of subchondral bone microarchitecture deterioration and sensory nerve innervation.

### 3.2 Background

Osteoarthritis (OA) is a leading cause of disability as the most common degenerative joint disorder, MMWR. Morbidity and mortality weekly report. 2009, and chronic pain is the most prominent symptom of osteoarthitis (OA), affecting nearly 40 million people in the US. Peat et al., 2001. Pain itself is also a major risk factor for the development of future functional limitation and disability in OA patients. Lane et al., 2010. Unfortunately, OA pain treatment remains challenging and represents a large unmet medical need. It is not clear what causes OA pain, and currently there is no effective way to relieve it. Available therapies (NSAIDs, steroids, visco-supplementation such as intra-articular injection of hyaluronic acid) only alleviate mild joint OA pain. Geba et al., 2002; Karlsson et al., 2002.

Relief from chronic OA pain remains an unmet medical need and still major reason for seeking surgical intervention. Despite the efforts, the origins of pain and its molecular mechanisms remain poorly understood. Significant efforts have been focused on synovium inflammation mediated sensitization of sensory neurons. Malfait and Schnitzer, 2013. Yet, OA pain can develop at very early stages without inflammation and independently of progressive cartilage degeneration. Many asymptomatic patients have osteoarthritic radiographic changes while other patients have OA pain with no radiographic indications. Bedson and Croft, 2008: Dieppe and Lohmander, 2005: Hannan et al., 2000. Some patients with bilateral radiographic OA yet present with unilateral knee pain. Far less attention has been paid to subchondral bone than to synovium, and the investigations into the mechanism how subchondral bone remodeling generates OA pain are lacking

The sources and mechanisms for OA pain remain unclear. Multiple tissues including synovium, Kc et al., 2016, ligament, Ikeuchi et al., 2012, and meniscus, Mapp and Walsh, 2012, were suggested to be the sites where pain is originated. In particular, significant efforts have focused on synovium inflammation (synovitis) and cartilage degeneration. Malfait and Schnitzer, 2013. Intriguingly, subchondral bone marrow edema-like lesions (BMLs) are highly correlated with OA pain. Yusuf et al., 2011; Kwoh, 2013. Zoledronic acid, which inhibits osteoclasts activity, reduces the BML size and concomitantly alleviates pain. Laslett et al., 2012. Furthermore, OA patients report rapidly pain relief after removal of partial subchondral bone with deteriorated cartilage by total knee replacement. Isaac et al., 2005; Reilly et al., 2005.

Subchondral bone remodeling is increased during OA progression. Zhen and Cao, 2014. It is shown that osteoclasts initiate aberrant bone remodeling and increase the secretion of netrin-1, which triggers abnormal sensory nerve innervation in the subchondral bone and causes OA pain. Subchondral bone marrow edema-like lesions (BMLs) are highly correlated with pain in OA patients. Kwoh, 2013; Laslett et al., 2012.

Analysis of the NIH Osteoarthritis Initiative (OAI) data also suggested that patients taking bisphosphonate experienced significantly reduced knee pain at year 2 and 3. Laslett et al., 2014. Bone homeostasis is maintained by temporal-spatial activation of TGF to couple bone resorption and formation. Zhen and Cao, 2014, in which subchondral bone is maintained in a native microarchitecture with blood vessels and nerves intertwined under normal condition. However, excessive active TGFβ in the subchondral bone induces aberrant bone remodeling at the onset of OA to promote its progression. Specifically, high levels of active TGFβ recruit Nestin+ MSCs and Osterix+ osteoprogenitors in clusters, leading to abnormal bone formation and angiogenesis. Zhen et al., 2013. Increased osteoclast activity is associated with angiogenesis at the onset of OA. Zhen et al., 2013; Xie et al., 2014. Since nerves and blood vessels develop together, the increased osteoclast activity likely induces abnormal sensory nerve innervation in the subchondral bone for the OA pain.

Parathyroid hormone (PTH), an FDA-approved anabolic agent for osteoporosis, regulates bone remodeling and calcium metabolism. Qiu et al., 2010; Pfeilschifter et al., 1995; Tang et al., 200) Woolf, 2011. The parathyroid gland, the main production site of the PTH, evolved in amphibians, Mease et al., 2011, and represents the transition of aquatic to terrestrial life, Suokas et al., 2012, adapting terrestrial locomotion from aquatic vertebrates. PTH is the hormone that PTH is demonstrated to induce cartilage regeneration for injury-induced OA, Sampson et al., 2011, Intermittent PTH stimulates subchondral bone and articular cartilage repair in the treatment of focal osteochondral defects. Orth et al., 2013.

The PTH is also shown to prevent the deterioration of the subchondral bone and cartilage degeneration during OA. Yan et al., 2014. It has been shown that PTH is demonstrated to interact with locally osteopotric factors to orchestrate with an anabolic signaling network of the coupling of bone resorption and formation. Qiu et al., 2010; Pfeilschifter et al., 1995. TGF-β elicits its cellular response through the ligand-induced formation of a heteromeric complex containing TGF-β types I (TβRI) and II (TβRII) kinase receptors. Tang et al., 2009; Wrana et al., 1992.

Several lines of evidence have indicated that PTH and TGF-β work in concert to exert their physiological activities in bone. PTH induces endocytosis of PTH1R with TβRII as a complex and signaling of both PTH and TGFβ is coordinately regulated during endocytosis. Qiu et al., 2010.

This study investigates whether iPTH could attenuate pain by modifying OA as it has positive effect on both OA subchondral bone and articular cartilage. It was found that PTH reduces OA pain and attenuates progression of OA by preventing subchondral bone deterioration and cartilage degeneration in OA mice. PTH reduces sensory nerve innervation in the subchondral bone and OA pain through maintaining subchondral bone micro-architecture of sustaining of coupled bone remodeling.

### 3.3 Results

3.3.1 iPIH Attenuates Osteoarthritic Pain and Joint Degeneration

To investigate the effect of PTH on OA pain, PTH was administered subcutaneously in OA mice post DMM for two months. Pain behavior tests were performed including paw withdrawal threshold (PWT), pressure application measurement (PAM) and gait analysis at different timepoints (FIG. 3-1A). PWT decreased in 1- and 2-weeks post DMM and controls, and the sham group gradually returned to the initial base level (FIG. 3-1A). iPTH significantly increased PWTs in DMM mice from 3 weeks relative to DMM vehicle group and the increase was maintained through 8 weeks. Similarly, iPTH also significantly increased PAM relative to vehicle group (FIG. 3-1B). Catwalk gait analysis showed that the ratio of left hind/right hind (LH/RH) paw intensity, contact area and swing speed decreased in vehicle DMM mice relative to sham-operated mice 8 weeks post DMM surgery, and again, iPTH significantly increased the changes in Catwalk (FIG. 3-1C and FIG. 3-1D). Fast green and Safranin O staining of joint sections showed that proteoglycan started loss in cartilage 2 weeks post DMM in vehicle group and further aggravated at 8 weeks relative to sham-operated group (FIG. 3-1E). iPTH reduced cartilage degeneration in DMM group and significantly improved Osteoarthritis Research Society International (OARSI) scores, Pritzker et al., 2006, relative to vehicle-treated DMM joint (FIG. 3-1F). Moreover, iPTH also reduced the percentage of MMP13⁺ and type X collagen⁺ chondrocytes in DMM mice relative to vehicle group, indicating inhibition of chondrocyte degeneration (FIG. 3-1, FIG. 3-G, FIG. 3-H, FIG. 3-I and FIG. 3-J). Taken together, these data suggest that iPTH-reduced pain is associated with attenuation of cartilage degeneration in DMM OA mice.

3.3.2 iPTH Induces Regression of Sensory Nerve Fibers in Subchondral Bone in OA Mice

To examine the potential mechanism of PTH-reduced OA pain, the effect of iPTH on sensory nerve innervation in subchondral bone was examined. The immunostaining of subchondral bone sections revealed that calcitonin gene-related peptide (CGRP)+ and substance P (SP)⁺ sensory nerve fibers significantly increased in vehicle-treated DMM mice relative to sham operated mice, and iPTH ameliorated them(FIG. 3-2A, FIG. 3-2B and FIG. 3-2C). Based on a newly proposed classification of sensory neurons, Usoskin et al., 2015, another 3 markers of nociceptive neurons NF200, P2X2, and PIEZO2 also were stained. The density of P2X3⁺, PIEZO2⁺, and NF200⁺ nociceptive fibers also increased in subchondral bone of vehicle-treated DMM mice relative to sham-operated mice while iPTH treatment significantly those fibers innervation (FIG. 3-2A. FIG. 3-20, FIG. 3-2E and FIG. 3-2F). In addition, there was also similar increase in PGP9.5⁺ nerve endings in DMM vehicle group, PTH treatment induces PGP9.5⁺ nerve endings decrease to be comparable to sham group (FIG. 3-2A and FIG. 3-2G). Furthermore, the immunostaining of CGRP⁺ sensory nerve fiber and PGP 9.5⁺ nerve fiber in the joint synovium were also analyzed and showed that there were no significant differences in density of CGRP⁺ and PGP9.5⁺ nerve between DMM PTH group and DMM vehicle group, although both of them significantly increased compared with synovium of sham-operated DMM mice (FIG. 3-2, FIG. 3-2H, FIG. 3-2I and FIG. 3-2J). Together, these findings suggest that intermittent PTH treatment may ameliorate OA pain by inhibition of sensory nerve innervation in subchondral bone.

3.3.3 iPTH Sustains Subchondral Bone Micro-Architecture

To examine PTH effect on subchondral bone changes in OA, PTH was administered daily subcutaneously in mice post DMM for 8 weeks and analyzed the effect over time. iPTH sustained the micro-architecture of tibial subchondral bone after DMM relative to sham-operated mice and DMM vehicle mice as determined by μCT analysis (FIG. 3-3A, top). iPTH significantly inhibited change of subchondral bone plate (SBP), structure model index (SMI), trabecular pattern factor (Tb.Pf) and total volume of pore space Po.V(tot) relative to DMM vehicle group (FIG. 3-3B, FIG. 3-C, FIG. 3-D and FIG. 3-E). In parallel, iPTH ameliorated the increased expression of COX2 determined by immunohistochemical analysis and PGE2 concentration of subchondral bone determined by Elisa in DMM vehicle group (FIG. 3-3F and FIG. 3-3G). iPTH reduced abnormal localization, as most osteoid were largely found on the bone surface, like sham-operated group, where new formed osteoid were observed in subchondral bone marrow in DMM vehicle group (FIG. 3-3H). Uncoupled bone remodeling was rescued by the PTH compared to the DMM vehicle group in fluorescent double labeling experiment. (FIG. 3-3I) These results indicated that PTH play an important role in sustaining coupled subchondral bone remodeling in OA. Taken together, these data validated that PTH attenuates OA progression by decelerating subchondral bone deterioration.

3.3.4 iPTH Attenuate Elevated Active TGF-§ Signaling by Endocytosis of TGFβIIR

To explore the potential mechanism of PTH restoring coupled subchondral bone remodeling, the effect of PTH on osteoblast-lineage cell was detected. Immunostaining of nestn⁺ and osterix⁺ osteoprogenitors were largely located on the bone surface in sham group and DMM PTH group while nestin⁺ and osterix⁺ osteoprogenitors in subchondral bone marrow dramatically increased in DMM vehicle group (FIG. 3-4A, FIG. 3-4B and FIG. 3-4C). The immunostaining of pSmad2/3 revealed that the number of pSmad2/3⁺ cell in subchondral bone was significantly decreased in DMM PTH group (FIG. 3-4D and FIG. 3-4F). The tartrate-resistant acid phosphatase-positive (TRAP) staining showed that osteoclast significantly increased in number in the subchondral bone post DMM relative to sham group, and the TRAP⁺ osteoclast further increased in subchondral bone in DMM PTH group (FIG. 3-4E and FIG. 3-4G). Furthermore, Elisa of active TGFβ1 of serum revealed that iPTH induced a further increase of the active TGFβ1 concentration relative to DMM vehicle group while a lower level of active TGFβ1 was detected in sham-operated group (FIG. 3-4H). Endocytosis of PTH1R has been shown to integrate signals of TGFβ pathways. Qiu et al., 2010.

TβRII was largely localized at mesenchymal stromal cell (MSC) membrane and the amount of cell-surface TβRII was decreased significantly after stimulated with PTH (FIG. 3-4I). Moreover, the immunostaining of pSmad2/3 showed that PTH decreased TGFβ1-induced phosphorylation and the nuclear accumulation of Smad2/3 relative to TGFβ1 stimulation alone (FIG. 3-4J). Collectively, high concentration of active TGFβ1 leading to aberrant subchondral bone formation, was partially prevented by PTH induced endocytosis of TGFβIIR.

### 3.3.5 Delayed IPTH Attenuates Progressive Osteoarthritic Pain and Joint Degeneration

To examine the effect of PTH treatment on progressive OA, a 4-week treatment with vehicle or PTH was initiated 4 weeks post sham surgery or DMM. This delayed regimen was employed to examine the impact of treatment in the clinical situation where the therapy is initiated after a diagnosis of OA. Delayed regime of iPTH significantly inhibited decrease of PWTs and PAMs in DMM mice relative to DMM vehicle group (FIG. 3-5A and FIG. 3-5B). Catwalk gait analysis showed that iPTH inhibited decreased of the ratio of left hind/right hind (LH/RH) paw intensity, contact area and swing speed decreased in DMM vehicle mice (FIG. 3-5C) The immunostaining of subchondral bone sections revealed that iPTH significantly ameliorated the increased density of CGRP⁺ and substance SP⁺ sensory nerve fibers in DMM vehicle mice (FIG. 3-5D, FIG. 3-5E, and FIG. 3-5F).The degeneration of articular cartilage was attenuated with delayed iPTH relative to DMM vehicle mice, as reflected by SOFG staining and OARSI scores (FIG. 3-5G). Similar to immediate iPTH treatment, delayed iPTH significantly prevented deterioration of micro-architecture of subchondral bone relative to DMM vehicle group (FIG. 3-5H top). The μCT analysis showed that iPTH treatment decreased SPB.Th, SMI, Tb.Pf and tot relative to DMM vehicle group (FIG. 3-5I). The immunochemistry staining and analysis showed that iPTH reduce the number of COX2⁺ cells as compared to DMM vehicle group (FIG. 3-H bottom and FIG. 3-5J). Consistently, the Elisa analysis demonstrated that increased PGE2 concentration of the subchondral bone in DMM vehicle group was significantly decreased by iPTH (FIG. 3-5J). Furthermore, iPTH significantly decreased the number of osterix⁺ progenitor clusters in bone marrow cavity of DMM vehicle group (FIG. 3-5K). Taken together, PTH attenuated progressive OA and subsequent sensory nerve innervation for osteoarthritic pain by preventing deterioration of subchondral bone microstructure.

### 3.3.6 Knockout of P7H1R in ASCs Inhibits PTH Induced Osteoarthritic Pain Relief and Joint Degeneration Prevention

To validate PTH attenuate OA progression and OA pain through maintaining subchondral bone microarchitecture after DMM, conditional knockout of PTH1R was induced in nestin+ MSCs of DMM mice. Nestin-CreTMER::PTH1Rfl/fl (PTH1R^{-/-}) mice were injected with tamoxifen to delete PTH1R in the nestin⁺ MSCs, unresponsive to PTH to eliminate PTH-induced endocytosis of TGFβIIR. There were no significantly difference in osteoarthritic pain reflected by PWT and PAM and gait analysis between DMM PTH and vehicle PTH1R^{-/-} mice while iPTH effectively attenuated OA pain reflected by improved PAM and PWT and gait analysis for DMM PTH1R^{fl/fl} (PTH1R^{+/+}) mice relative to DMM vehicle PTH1R^{+/+} group (FIG. 3-6A, FIG. 3-6B, and FIG. 3-6C). Moreover, CGRP⁺, SP⁺, P2X3⁺, NF200⁺ and PIZEO2⁺ sensory nerve fiber and PGP9.5⁺ nerve fibers in subchondral bone of DMM PTH PTH1R^{-/-} mice were comparable to that of DMM vehicle PTH1R^{-/-} mice (FIG. 3-6D and FIG. 3-6E). Regarding to PTH1R^{-/-} mice, iPTH significantly decreased the density of sensory nerve fiber relative to DMM vehicle group (FIG. 3-6D and FIG. 3-6E). However, there was no statistically significant difference in density of both CGRP⁺ and PGP9.5⁺ nerve fiber between DMM PTH and vehicle group for either PTH1R^{-/-} or PTH1R^{+/+} mice (FIG. 3-6F and FIG. 3-6G).

Similarly, iPTH failed to prevent joint degeneration in DMM PTH1R^{-/-} mice. The SOFG staining showed that proteoglycan loss in articular cartilage was not prevented in the DMM PTH PTH1R^{-/-} mice (FIG. 3-7A top and FIG. 3-7B). The micro-architecture including SMI, Tb.Pf. SBP.Th and tot were not significantly improved in DMM PTH PTH1R^{-/-} mice (FIG. 3-7A middle and FIG. 3-7C), again iPTH attenuated them in DMM PTH1R^{+/+} mice. Similarly, iPTH decreased COX2 expression determined by immunochemistry staining and subchondral bone PGE2 concentration determined by ELISA in DMM PTH1R^{+/+} mice relative DMM vehicle PTH1R^{+/+} mice, iPTH failed to decrease them in PTH1R^{-/-} mice (FIG. 3-7A bottom, FIG. 3-7D and FIG. 3-7E). The number of pSmad2/3⁺ cell in subchondral bone in DMM PTH PTH1R^{-/-} mice was comparable to DMM vehicle PTH1R^{-/-} mice (FIG. 3-7F). Subsequently, iPTH failed to decrease the number of nesin⁺ osteoprogenitors and osterix⁺ in the subchondral bone marrow failed to achieve significant difference between PTH and vehicle treated DMM PTH1R^{-/-} mice. Regarding to PTH1R^{+/+} mice, iPTH significantly decreased pSmad2/3⁺ cell in number and the number of nesin⁺ osteoprogenitors and osterix⁺ in the subchondral bone marrow relative to DMM vehicle group (FIG. 3-7G and FIG. 3-7H). The results validate that PTH reduce active TGF β signaling in the subchondral bone to prevent osteoarthritis, and further suggested that the role of PTH on preventing osteoarthritic pain and sustaining micro-architecture is distinct from its role in articular cartilage.

### 3.4 Discussion

The current routine treatments of OA including non-steroidal anti-inflammatory drugs and analgesics have limited therapeutic effect. Hochberg et al., 2012. These drugs are palliative treatment with progressive pathological joint changes and unsustained pain relief. Surgical joint replacement is the only alternative for end-stage of OA. Thomas et al., 2009. The only purportedly disease-modifying therapy for OA is to provide cartilage proteoglycan components via dietary or via intra-articular injection. Zhang et al., 2008: Vangsness et al., 2009; Brzusek and Petron, 2008: Brander and Stadler, 2009.

No consensus, however, is reached on the efficacy of oral ingestion of aggrecan sugar moieties, Zhang et al., 2008, and intra-articular injection of hyaluronic acid have pain relief only up to 6 months. Brander and Stadler, 2009. Thus, the development of an effective disease-modifying treatment of the OA joint with pain relief is urgently needed. In the current study, it was found that PTH could be a potential disease-modifying therapy of OA, considering that PTH reduce OA pain and attenuate progression of OA by preventing subchondral bone deterioration and cartilage degeneration. The OA pain relief and prevention of progressive OA were due to PTH-induced maintain of subchondral bone micro-architecture by restoration of coupled bone remodeling and prevention of nerve innervation.

In this study, no significant protection of articular cartilage degeneration was observed when intermittent PTH was applied to Nestin-CreTMER::PTH1R^{fl/fl} DMM mice compared with PTH-treated DMM PTH1R^{fl/fl} mice, suggesting that PTH-induced sustain of subchondral bone microarchitecture is critical for protection of articular cartilage during OA. Specifically, the decreased density of CORP⁺ sensory nerve in subchondral bone in Dmp1-Rankl^{fl/fl} and Trap-Ntn^{fl/fl} is obviously associated with significant pain relief reflected by Catwalk and Von Frey. Zhu et al., 2019, indicating that sensory nerve of subchondral bone might be an important origin of OA pain. OA pain of joint (activation and sensitization of nociceptive neurons) occurs episodically during movement and loading of the joint and this pain may be evoked by specific activity such as pinch-evoked pain hypersensitivity. Felson, 2009.

The primary knee hyperalgesia of OA has recently been measured by withdrawal threshold of direct knee press using a Pressure Application Measurement (PAM) device in DMM mice. Miller et al., 2017. The secondary hyperalgesia of knee developed after OA is also suggested to be measured by mechanical hypersensitivity of hind paw. Zhu et al., 2019. Sing of central sensitization, manifesting as pain hypersensitivity, has been described, such as mechanical allodynia (pain caused by a stimulus that does not normally evoke pain), reduced pain pressure thresholds, and enhanced temporal summation. Woolf, 2011: Mease et al., 2011; Suokas et al., 2012.

Various and complementary method are carried out for global measure of OA pain in this study. The development of pain behavior involves gait alterations due to pain during activities that shouldn't cause pain, such as pain during walking or loading. The movement-provoked pain behaviors reflected by gait analysis is effectively inhibited in PTH-treated DMM mice. The better mechanical hypersensitivity reflected by Von Frey filament stimulation applied to operated hind paw and better performance of operated hind paw in PTH-treated DMM mice are consistent with lower density of sensory nerve in subchondral bone of PTH-treated DMM mice.

Articular cartilage and subchondral bone forms as a mechanical and biological functional unit. Currently. OA is considered as a disease of the whole joint and the capacity of cross talk between articular cartilage and subchondral bone is enhanced with alteration of subchondral bone in OA. Zhu et al., 2019: Felson, 2009. The PTH mainly affected subchondral bone and articular cartilage of the joint, little literature established that PTH have direct effect on synovium, muscle or other soft tissues. It was commonly suggested that OA pain mainly originate from synovium, ligament. Kc et al., 2016; menisci, Ashraf et al., 2011; subchondral bone, Reimann and Christensen, 1977; and muscle and joint capsule. Hirasawa et al., 2000.

The degeneration of articular cartilage, especially hyaline cartilage, unlikely itself gives rise to pain, because cartilage is normally not innervated. Schaible, 2012. But it was reported that osteochondral junction was innervated by sensory nerve originated from osteochondral junction (or subchondral bone). Sun et al., 2007. It was also validated that there was no correlation between joint nociception and articular damage. McDougall et al., 2009. That suggested that PTH-induced chondro-protective and chondro-regenerative effect on articular cartilage has little relationship with pain relief during OA treatment. Additionally, PTH is also expected to promote bone resorption of old or micro-damaged subchondral bone of OA to provide basis for new bone formation than sustain subchondral bone micro-architecture.

TrkA-positive nerve fibers were observed at innervated sites of incipient primary ossification, coincident with NGF expression in cells adjacent to centers of incipient ossification. Tomlinson et al., 2016. That suggest that nerve sustains bone homeostasis by locating adjacent bone surface in normal condition. Intermittent PTH spatially redistributes smaller blood vessels not larger vessels closer to bone-forming site for providing a favorable microenvironment for growth. Roche et al., 2014; Prisby et al., 2011.

Altogether, the PTH induced alteration of vessel suggested that remodeling of bone vascular morphology is necessary for PTH osteoanabolic effect and its hemodynamic function. Recently, a subgroup of capillary, named type H vessel, with high level of CD31 and endomucin in the murine skeletal system was identified and found to mediated growth of bone vasculature, sustain perivascular osteoprogenitors and coupled angiogenesis to osteogenesis. Kusumbe et al., 2014.

Consistently, the type H vessel intensity significantly increased in subchondral bone of PTH treated DMM mice than that of vehicle treated DMM mice. Osteoblast-derived VEGF were required for coupling of angiogenesis and osteogenesis by stimulating recruitment of blood vessels and osteoclast, to make sure blood vessel provide favorable microenvironment for osteogenesis. Hu and Olsen, 2016.

Type H vessel was expected to locate around bone surface in the subchondral bone marrow of PTH treated DMM mice, which also suggested that osteoprogenitor cell was recruited to bone resorption bone surface after PTH treated with type H vessel providing molecular microenvironment for coupled angiogenesis and osteogenesis. Considering blood vessel and nerve fiber course was often alongside one another due to sharing similar mechanism about wiring neural and vascular networks. Carmeliet and Tessier-Lavigne, 2005, it was thought that nerve fiber may undergo similar remodeling to small vessel after PTH treatment, that may one of reasons of PTH induced pain relief in osteoarthritis.

PGE2 secreted osteoblast that sensed lower bone volume control bone homeostasis and promote regeneration by sensory nerve. Chen et al., 2019. The larger cavity of subchondral bone due to less loading stress (biomechanical adaptation of the bone) during OA was sense by local osteoblast, which secrets PGE₂ to sustain bone homeostasis. Both peripheral and spinal hyperexcitability related to pathophysiological pain states were inhibited even reversed during development of inflammation and established hyperexcitability by the selective cyclooxygenase-2 (COX-2) inhibitors. Woolf and Salter, 2000; Telleria-Diaz et al., 2010.

The higher concentration of PGE2 of subchondral bone, as an inflammation factors, may be one of main reasons of OA pain. The PTH-sustained subchondral bone micro-architecture with even cavity maybe associated with lower concentration of PGE₂, resulted in relatively mild OA pain. Additionally, intermittent PTH promoted bone formation and boosted bone mass by endocytosis of LRP6/PTH1R complex, enhancing BMP signaling. Yu et al., 2012. Subsequently, PTH-induced bone formation then inhibited PGE2 formation due to osteoblast sensing increased bone volume.

Subchondral bone osteoblasts of osteoarthritis are resistant PTH stimulation, which could be attributed to reduced expression or altered recycling process of PTH1R. Hilal et al., 1998. That is consistent with reported lower PTH receptor level in OA compared to normal osteoblast. Hilal et al., 2001. Endogenous PGE₂ in subchondral bone could repress PTH-dependent response in OA osteoblasts, further contribute to abnormal bone remodeling and bone sclerosis in OA. The blunted PTH signaling due to elevated PGE2 and IGF1, Hilal et al., 2001, signaling during OA partially explained why there only a slightly increased in BV/TV of subchondral bone in PTH-treated DMM mice relative to vehicle-treated DMM mice. Conversely, decrease expression of PGE2 of OA subchondral bone after PTH treatment compared to vehicle OA mice might partially restore normal PTH signaling, which was in part due to relatively decreased inflammation and PTH-induced anabolic effect and sustained microarchitecture of subchondral bone where PTH retards osteoarthritis progression.

Cell senescence of osteoblasts and osteocytes have been identified with trabecular and cortical bone and cartilage in older animals. Farr et al., 2016: Philipot et al., 2014. Given the vital role of these cells in bone remodeling and joint function, the accumulation of these senescent cells contributes to the promotion of OA. Excessive TGF-β/Smad activation is one of the predominant pathways that accelerate damage-induced and developmentally cellular senescence. Rapisarda et al., 2017; Lyu et al., 2018. TGF-β signaling inhibition by PTH during OA may contribute to reduction of cellular senescence to attenuate OA progression.

PTH was reported to provoke early osteoarthritis by induced alteration of normal subchondral bone micro-architecture. Orth et al., 2014. The PTH-induced altered structural parameters of subchondral bone cause thickening of the calcified layer, leading to osteoarthritic cartilage degeneration. A previous study revealed that normal concentration of active TGF-β, as a coupling factor of bone remodeling, induces migration of bone marrow MSCs to bone resorption site for bone formation. Tang et al., 2009.

High concentrations of active TGF-β1 signaling in the subchondral bone leads to aberrant bone remodeling, which is a key step in the pathogenesis of OA. Zhen et al., 2013. Excessive TGF β signaling of subchondral bone MSC in OA joint was inhibited to approach normal condition by PTH-induced endocytosis where coupled bone remodeling was partially restored, while the PTH-induced osteoarthritis of normal femoral joint may be attributed to disturbance of couple bone remodeling due to normal TGF β signaling inhibited by PTH. Furthermore, abnormally low mineralization (becomes sclerotic although hypomineralized) of subchondral bone during osteoarthritis was reversed in part by PTH anabolic effect to maintain relative normal cartilage stress, Grynpas et al., 1991, while abnormally higher mineralization of normal subchondral bone induced by PTH may accelerate cartilage degeneration.

The development of osteophytes in the joint margins is a key feature of osteoarthritis, especially in DMM model, Wright et al., 2009. Considering bone anabolic effects induced by PTH-PTH1R signaling, there might be a potential increased incidence of osteophyte formation when PTH was applied in the treatment of osteoarthritis. Three-dimensional reconstructions generated from micro-CT data of all groups was collected for the detection of osteophyte formation. Interestingly, PTH-treated mice did not lead to an increased incidence of osteophyte formation during osteoarthritis compared to the vehicle-treated control group.

In the current study, the subcutaneously concentration of PTH (40 μg/kg/day) is effective for the treatment of OA in DMM mice. Based on published literatures showing that the effective does of PTH for the treatment of osteoarthritis ranged from subcutaneous injection (40 μg/kg), Sampson et al., 2011, to intraperitoneally injection (80 μg/kg) for mice. Dutra et al., 2017, which was comparable to the concentration here. Notably, the dose of 40 μg/kg/day for the treatment of DMM mice in this study was significantly higher than accepted and optimal concentration for other species, such as guinea pig(15 μg/kg/day), Yan et al., 2014; rabbit (10 μg/kg/day), Orth et al., 2014; and rat (15 μg/kg/day). Ma et al., 2017. It was anticipated that there was a species-associated shift for effective concentration of PTH supporting the effect of PTH-induced OA pain relief and restoration of coupled bone remodeling then the further attenuation of OA progression.

### 3.5 Methods

### 3.5.1 Mice

C57BL6J mice (WT mice. Stock number: 000664) were purchased from Jackson Laboratory, 10-weeks old male mice were anesthetized with xylazine and ketamine and then transected meniscotibial ligament that connects lateral side of medial meniscus with intercondylar eminence of tibia to induce mechanical instability associated osteoarthritis on the left knee, Sham operations of DMM were done on independent mice. For immediate treatment group, beginning 3 days after surgery, PTH (40 μg/kg/day) or the equivalent volume of vehicle (PBS) was injected subcutaneously daily until sacrifice. For the time-course experiment, operated mice were euthanized at 2, 4, 8, and 12 weeks postoperatively (n=8 per treatment group). Regarding to delayed treatment group, PTH (40 μg/kg/day) or the equivalent volume of vehicle (PBS) was injected subcutaneously daily from 4 weeks to 8 weeks after surgery, then these mice were sacrificed (n=8 per treatment group).

Nestin-creERT2 mice (Stock number: 016261) and R26R-EYFP (Stock number:006148) were purchased from Jackson Laboratory. Mice with floxed PTH1R (PTH1R^{flox/flox}) were obtained from the lab of Dr. Henry Kronenberg. Kobayashi et al., 2002.

The genotype of transgenic mice was determined by PCR analyses of genomic DNA isolated from mouse tails. The floxed PTH1R allele was identified with primers lox1F (5′-TGGACGCAGACGATGTCTTTACCA-3′ (SEQ ID NO: 28)) and lox1R (5′-ACATGGCCATGCCTGGGTCTGAGA-3′ (SEQ ID NO: 29)). The genotyping for the creERT2 transgene was performed by PCR with the primers Cre F (5′-ACCAGAGACGGAAATCCATCGCTC-3′ (SEQ ID NO: 30)) and Cre R (5′-TGCCACGACCAAGTGACAGCAATG-3′ (SEQ ID NO: 31)). Nestin-creERT2:: PTH 1R flox/flox mice were generated by crossing Nestin-creERT2 mice with PTH1R flox/flox mice. Then these mice were backcross with PTH1R flox/flox mice to generate Nestin-creERT2::PTH1R flox/flox and PTH 1R flox/flox mice. DMM or sham operations were performed on 10-week old Nestin-creERT2::PTH1R flox/flox and PTH1R flox/flox male mice. Three days before surgery, the mice were treated with either 100 mg/kg body weight of tamoxifen or vehicle three times per week for 4 and 8 weeks. Additionally, the mice (n=8 per treatment group) were treated with either PTH (40 μg/kg/day) or the equivalent volume of vehicle (PBS) subcutaneously daily for 4 and 8 weeks, 3 days after surgery.

### 3.5.2. Cell Culture

Bone marrow stromal cell was obtained from 8-weeks old WT mice as described by Soleimani and Nadri, 2009. Cells (Passage 3-5) were maintained in Iscove's modified Dulbecco's medium (Invitrogen) supplemented with 10% fetal calf serum (Atlanta Biologicals), 10% horse serum (Thermo Scientific), and 1% penicillin-streptomycin (Mediatech). MSCs were cultured in 6 well plates at a density of 1.8×10⁵ cells per well, then MSC were starved for 6 h before treatment. Human PTH (1-34) and PTH (7-34) was purchased from Bachem, Inc. (Torrance, Calif.), 100 nM of or PTH(7-34), or 2 ng of TGF-β1, was used for cell treatment as indicated.

### 3.5.3. ELISA

The concentration of active TGF-β1 in the serum was determined using the TGF-β1 ELISA Development kit (human: R&D Systems. DB100B; mouse: R&D Systems, MB100B) and following the manufacturer's instructions.

### 3.5.4. Histochemistry, Immunohistochemistry and Histomorphometry

After mice were killed by carbon dioxide (CO₂) inhalation and perfused by phosphate buffer saline (PBS) and fixed by 10% buffered formalin via the left ventricle, the left knee joints were resected and fixed in 10% buffered formalin for 24 hours, decalcified in 0.5M ethylenediamine tetraacetic acid (pH 7.4) for 21 days, and embedded in paraffin or Optimal Cutting Temperature Compound (O.C.T. compound, VWR, 25608-930). The blocks were sectioned at 4 μm intervals using a Paraffin Microtome (for paraffin blocks) or 30 μm intervals using a using a Microm cryostat (for frozen blocks). Four-μm sagittal oriented sections of the operated knee joint medial compartment for hematoxylin and eosin (H&E) staining and safranin o (Sigma-Aldrich, S2255) and fast green (Sigma-Aldrich, F7252) staining. Tartrate resistant acid phosphatase staining was performed using the manufacturer's protocol (Sigma-Aldrich, 387A-1KT), followed by counterstaining with Methyl Green (Sigma-Aldrich, M884). Immunostaining including immunohistochemistry and immunofluorescence were performed using standard protocol. The tissue sections were incubated with primary antibodies to mouse pSmad2/3 (ThermoFisher Scientific, 444-244G, 1:100), mouse osterix (Abcam, ab22552, 1:200), mouse CD31 (Abcam, ab28364, 1:100), mouse endomucin (Santa Cruz, V.7C7, 1:50), mouse osteocalcin (Abcam, ab93876, 1:200), mouse nestin (Ayes Labs, Inc., 1:300, lot NES0407), mouse CGRP 0, mouse Substance P ( ), and mouse PGP9.5 ( ) overnight at 4° C. in a humidifier chamber. The sections were washed three times with Tris-buffered saline. For immunohistochemical staining, the slides were incubated with secondary antibodies in blocking solution at room temperature for 1 hours, and subsequently Chromogenic Substrates (Dako, K3468) was used to detect the immunoactivity, followed by counterstaining with hematoxylin (Sigma-Aldrich, H9627). For immunofluorescence staining, slides were incubated with secondary antibodies conjugated with fluorescence at room temperature for 1 hour, while avoiding light and mounted on slides with Prolong Gold Mounting Reagent with DAPI (Life Technologies, P36935). Isotype-matched controls, such as polyclonal rabbit IgG (R&D Systems, AB-105-C) and monoclonal rat IgG2A (R&D Systems, 54447), and polyclonal goat IgG (R&D Systems, AB-108-C) were used as negative controls under same condition and concentration. A Zeiss 780 confocal microscope or an Olympus DP72 microscope (Microscope Camera, Olympus, Tokyo, Japan) was used for image capture. Quantitative histomorphometric analysis was conducted in a blinded fashion with Image-Pro Plus Software version 6.0 (Media Cybernetics Inc., Rockville, Md.). The numbers of positive stained cells in five random visual fields of five sequential sections per mouse in each group were counted and normalized to the number per millimeter of adjacent bone surface (for TRAP staining quantification) or per square millimeter of bone marrow. For type H vessel and nerve quantification, the percentage area of positive staining was calculated by measuring the positive area and normalized to that of sham mice per (set to 1) in each group. Quantifications were performed using ImageJ 1.48u4 software.

### 3.5.5 Micro-Computed Tomography (μCT)

The left knee joint was dissected from mice free of soft tissue and fixed overnight in 10% formalin, and then analyzed by high resolution μCT (Skyscan 1172). The scanner was set at a voltage of 65 kV and a current of 153 μA and a resolution of 9 μm per pixel. The images were reconstructed, analyzed for HO bone volume, and visualized by NRecon v1.6, CTAn v 1.9, and CTVol v2.0, respectively. Cross-sectional images of the tibiae subchondral bone. The region of interest was defined as the whole medial compartment of subchondral bone and cross-sectional sagittal image of the tibiae subchondral bone were used for three-dimensional histomorphometric analysis. A total of six consecutive image from medial tibial plateau were used for 3D reconstruction. Cross-sectional sigittal images of the tibiae subchondral bone were used to perform three-dimensional histomorphometric analysis. The following three-dimensional structural parameters were analyzed in this study: BV/TV: trabecular bone volume per tissue volume, SMI: structure model index. Tb.pf: trabecular pattern factor. SBP: subchondral bone thickness, and Tb.Th: trabecular thickness.

### 3.5.6 Gait Analysis

Detail automated analysis of gait was performed on walking mice using a “CATWALK” system (Noldus) pre-surgery, 2, 4, 6 and 8 weeks post-surgery. All experiments were performed at the same period of the day (12:00 PM-4:00 PM) and analyzed as previously reported method. Hamers et al., 2006; Hamers et al., 2001.

The recording was carried in a completely dark room with exception of the light from the computer screen. Briefly, mice were trained to cross the Catwalk walkway daily for 7 days before DMM or sham operation. During the test, each mouse was placed individually in the Catwalk walkway and allowed to walk freely and traverse form one side to the other side of the walkway glass plate. Light from an encased fluorescent lamp was emitted inside the glass plate and completely internally reflected. When the mouse paws contacted the glass plate, light was reflected down and the illuminated contact area was recorded with a high-speed color video camera positioned underneath the glass plate connected to a computer running Catwalk software v9.1 (Noldus). Comparison was made between the ipsilateral (left) and the contralateral (right) hind paw in each run of each animal at each time point. In the present study, the following parameters were analyzed: contact area; intensity and swing speed.

### 3.5.7 Von Frey

The 50% paw withdrawal threshold was measured by Von Frey hair algesiometry. Mice was habituated to elevated Plexiglas chambers and wire mesh flooring prior to assessments of allodynia. Then, ipsilateral hind paw mechano-sensitivity was measured by a modification of the Dixon up-down method. Dixon, 1980. Allodynia were evaluated by application of von Frey hair in ascending order of known bending force (force range: 0.07 g, 0.4 g, 0.6 g, 1 g, 1.4 g, 2 g, 4 g, or 6 g). The von Frey hair was applied perpendicular to the plantar surface of the hind paw (avoiding the toe pads) for 2-3 s, once a withdrawal response was occurred the paw was re-tested starting with the next descending von Frey hair until no response occurred. Four more measurements were made after the first difference were observed. The 50% PWT was determined by the following formula: 50% PWT=10^{Xf+kδ/}10000, where Xf is the exact value of the final test of von Frey hair, K is the tabular value for the pattern of the last 6 positive/negative responses, and S is the mean difference (in log units) between stimuli. The threshold force required to elicit withdrawal of the paw (median 50% withdrawal) was determined twice on each hind paw (and averaged) on each testing day, with sequential measurements separated by at least 5 min.

### 3.5.8. Statistics

All data were analysis were performed using SPSS 22.0 analysis software (SPSS Inc). The data are presented as the mean±standard deviation (SD). Unpaired two-tailed Student's t-test were used for comparison between two groups and one-way analysis of variance (ANOVA) followed by were used to determined significant difference between multiple groups. The level of significance as set at p<0.05. All inclusion/exclusion criteria were pre-established, and no samples or animals were excluded from the analysis. No statistical method was used to predetermine the sample size. The experiments were randomized. The investigators were not blinded to allocation during experiments and outcome assessment.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.
Ariga, K. et al. The relationship between apoptosis of endplate chondrocytes and aging and degeneration of the intervertebral disc. Spine 26, 2414-2420 (2001).
Ashraf S, Wibberley H, Mapp P I, Hill R, Wilson D, Walsh D A. Increased vascular penetration and nerve growth in the meniscus: a potential source of pain in osteoarthritis. Annals of the rheumatic diseases, 2011:70(3):523-529.
Battie, M. C., et al., Genetic and environmental effects on disc degeneration by phenotype and spinal level: a multivariate twin study. Spine (Phila Pa. 1976), 2008, 33(25): p. 2801-8.
Bedson J, Croft P R. The discordance between clinical and radiographic knee osteoarthritis: A systematic search and summary of the literature. Bmc Musculoskeletal Disorders, 2008; 9.
Benneker, L. M., et al., 2004 Young Investigator Award Winner: vertebral endplate marrow contact channel occlusions and intervertebral disc degeneration. Spine (Phila Pa. 1976), 2005, 30(2): p. 167-73.
Bian, Q. et al. Excessive Activation of TGF beta by Spinal Instability Causes Vertebral Endplate Sclerosis. Scientific Reports 6. doi:ARTN 2709310.1038/srep27093 (2016).
Bian, Q. et al. Mechanosignaling activation of TGFbeta maintains intervertebral disc homeostasis. Bone Res 5, 17008. doi:10.1038/boneres.2017.8 (2017).
Bikle, D. D., et al., Insulin-like growth factor I is required for the anabolic actions of parathyroid hormone on mouse bone. J Bone Miner Res, 2002, 17(9): p. 1570-8.
Boos, N. et al. Classification of age-related changes in lumbar intervertebral discs: 2002 Volvo Award in basic science. Spine 27, 2631-2644 (2002).
Borenstein, D. G. et al. The value of magnetic resonance imaging of the lumbar spine to predict low-back pain in asymptomatic subjects—A seven-year follow-up study. Journal of Bone and Joint Surgery-American Volume 83a, 1306-1311, doi:Doi 10.2106/00004623-200109000-00002 (2001).
Brander V A, Stadler T S. Functional improvement with hylan G-F 20 in patients with knee osteoarthritis. The Physician and sports medicine. 2009; 37(3):38-48.
Brown, M. F. et al. Sensory and sympathetic innervation of the vertebral endplate in patients with degenerative disc disease. J Bone Joint Surg Br 79, 147-153 (1997).
Brzusek D, Petron D. Treating knee osteoarthritis with intra-articular hyaluronans. Current medical research and opinion. 2008; 24(12):3307-3322.
Cai, G. Q. et al. Effect of Zoledronic Acid and Denosumab in Patients With Low Back Pain and Modic Change: A Proof-of-Principle Trial. Journal of Bone and Mineral Research 33, 773-782, doi:10.1002/jbmr.3376 (2018).
Canalis, E., et al., Insulin-like growth factor I mediates selective anabolic effects of parathyroid hormone in bone cultures. J Clin Invest, 1989, 83(1): p. 60-5.
Carmeliet P, Tessier-Lavigne M. Common mechanisms of nerve and blood vessel wiring. Nature. 2005:436(7048):193-200.
Chen H. Hu B, Lv X, et al. Prostaglandin E2 mediates sensory nerve regulation of bone homeostasis. Nature Communications. 2019; 10(1):181.
Clark, P. et al. MF498 [N-{[4-(5,9-Diethoxy-6-oxo-6,8-dihydro-7H-pyrrolo[3,4-g]quinolin-7-yl)-3-methylbenzyl]sulfonyl}-2-(2-methoxyphenyl)acetamide], a selective E prostanoid receptor 4 antagonist, relieves joint inflammation and pain in rodent models of rheumatoid and osteoarthritis. The Journal of pharmacology and experimental therapeutics 325, 425-434 (2008).
Cobos, E. J. et al. Inflammation-induced decrease in voluntary wheel running in mice: a nonreflexive test for evaluating inflammatory pain and analgesia. Pain 153, 876-884 (2012).
Coggeshall, R. E., Tate, S. & Carlton, S. M. Differential expression of tetrodotoxin-resistant sodium channels Naᵥ1.8 and Naᵥ1.9 in normal and inflamed rats. Neuroscience letters 355, 45-48 (2004).
Dieppe P A, Lohmander L S. Pathogenesis and management of pain in osteoarthritis. Lancet. 2005:365(9463):965-973.
Disease, G. B. D., 1. Injury. and C. Prevalence, Global, regional, and national incidence, prevalence, and years lived with disability for 328 diseases and injuries for 195 countries, 1990-2016: a systematic analysis for the Global Burden of Disease Study 2016. Lancet, 2017, 390(10100): p. 1211-1259.
Dixon W J. Efficient Analysis of Experimental Observations. 1980; 20(1):441-462.
Dudli, S., Fields, A. J., Samartzis, D., Karppinen, J. & Lotz, J. C. Pathobiology of Modic changes. European spine journal: official publication of the European Spine Society, the European Spinal Deformity Society. and the European Section of the Cervical Spine Research Society 25, 3723-3734 (2016).
Dutra E H, O'Brien M H, Gutierrez T, Lima A, Nanda R, Yadav S. PTH [1-34]-induced alterations predispose the mandibular condylar cartilage to mineralization. 2017; 20(S1):162-166.
England, S., Bevan, S. & Docherty, R. J. PGE2 modulates the tetrodotoxin-resistant sodium current in neonatal rat dorsal root ganglion neurones via the cyclic AMP-protein kinase A cascade. J Physiol 495 (Pt 2), 429-440 (1996).
Fahrleitner-Pammer, A., et al., Fracture rate and back pain during and after discontinuation of teriparatide: 36-month data from the European Forsteo Observational Study (EFOS). Osteoporos Int, 2011, 22(10): p. 2709-19.
Fan, Y., et al., Parathyroid Hormone Directs Bone Marrow Mesenchymal Cell Fate. Cell Metab, 2017, 25(3): p. 661-672.
Farr J N, Fraser D G, Wang H. et al. Identification of Senescent Cells in the Bone Microenvironment. 2016:31(11).1920-1929.
Felson DTJAR, Therapy. Developments in the clinical understanding of osteoarthritis. 2009; 11(1):203.
Fields, A. J., Liebenberg, E. C. & Lotz, J. C. Innervation of pathologies in the lumbar vertebral end plate and intervertebral disc. Spine Journal 14, 513-521, doi:10.1016/j.spinee.2013.06.075 (2014).
Forcet, C. et al. Netrin-1-mediated axon outgrowth requires deleted in colorectal cancer-dependent MAPK activation. Nature 417, 443-447 (2002).
Foster, N. E. et al. Prevention and treatment of low back pain: evidence, challenges, and promising directions. Lancet 391, 2368-2383, doi:10.1016/S0140-6736(18)30489-6 (2018).
Garcia-Cosamalon, J. et al. Intervertebral disc, sensory nerves and neurotrophins: who is who in discogenic pain? Journal of anatomy 217, 1-15 (2010).
Geba G P. Weaver A L, Polis A B, Dixon M E, Schnitzer T J, Grp V. Efficacy of rofecoxib, celecoxib, and acetaminophen in osteoarthritis of the knee—A randomized trial. Jama-Journal of the American Medical Association. 2002:287(1):64-71.
Global, regional, and national incidence, prevalence, and years lived with disability for 310 diseases and injuries, 1990-2015: a systematic analysis for the Global Burden of Disease Study 2015. Lancet 388, 1545-1602, doi:10.1016/S0140-673(416)31678-6 (2016).
Gruber, H. E., et al., Vertebral endplate and disc changes in the aging sand rat lumbar spine: cross-sectional analyses of a large male and female population. Spine (Phila Pa. 1976), 2007, 32(23): p. 2529-36.
Grynpas M D, Alpert B, Katz I, Lieberman I, Pritzker K P. Subchondral bone in osteoarthitis. Calcified tissue international. 1991; 49(1):20-26.
Gullbrand, S. E., et al., ISSLS Prize Winner Dynamic Loading-Induced Convective Transport Enhances Intervertebral Disc Nutrition. Spine (Phila Pa. 1976), 2015, 40(15): p. 1158-64.
Hamers D F P T, Koopmans G C, Joosten E A J. CatWalk-Assisted Gait Analysis in the Assessment of Spinal Cord Injury. 2006; 23(3-4):537-548.
Hamers F P, Lankhorst A J, van Laar T J, Veldhuis W B, Gispen W H. Automated quantitative gait analysis during overground locomotion m the rat: its application to spinal cord contusion and transection injuries. Journal of neurotrauma. 2001; 18(2):187-201.
Hancock, M. J. et al. Systematic review of tests to identify the disc, SIJ or facet joint as the source of low back pain. Eur Spine J 16, 1539-1550, doi:10.1007/s00586-007-0391-1 (2007).
Hand, R. A. & Kolodkin, A. L. Netrin-Mediated Axon Guidance to the CNS Midline Revisited. Neuron 94, 691-693 (2017).
Hannan M T. Felson D T, Pincus T. Analysis of the discordance between radiographic changes and knee pain in osteoarthritis of the knee. Journal of Rheumatology. 2000; 27(6):1513-1517.
Hartvigsen, J. et al. What low back pain is and why we need to pay attention. Lancet (London. England) 391, 2356-2367 (2018).
Hartvigsen, J., Christensen, K. & Frederiksen, H. Back pain remains a common symptom in old age, a population-based study of 4486 Danish twins aged 70-102. Eur Spine J 12, 528-534, doi:10.1007/s00586-003-0542-y (2003).
Hilal G, Martel-Pelletier J. Pelletier J P, Ranger P, Lajeunesse D. Osteoblast-like cells from human subchondral osteoarthritic bone demonstrate an altered phenotype in vitro: possible role in subchondral bone sclerosis. Arthritis and rheumatism. 1998; 41(5):891-899.
Hilal G, Massicotte F, Martel-Pelletier J, Fernandes J C, Pelletier J P, Lajeunesse D. Endogenous prostaglandin E2 and insulin-like growth factor 1 can modulate the levels of parathyroid hormone receptor in human osteoarthritic osteoblasts. Journal of bone and mineral research:the official journal of the American Society for Bone and Mineral Research. 2001:16(4) 713-721.
Hirasawa Y, Okajima S. Ohta M, Tokioka T. Nerve distribution to the human knee joint: anatomical and immunohistochemical study. International orthopaedics. 2000, 24(1):1-4.
Hochberg M C, Altman R D, April K T, et al. American College of Rheumatology 2012 recommendations for the use of nonpharmacologic and pharmacologic therapies in osteoarthritis of the hand, hip, and knee. Arthritis care & research. 2012:64(4):465-474.
Hu K. Olsen B R. Osteoblast-derived VEGF regulates osteoblast differentiation and bone formation during bone repair. The Journal of clinical investigation. 2016:126(2):509-526.
Hurri, H. & Karppinen, J. Discogenic pain. Pain 112, 225-228, doi:10.10161j.pain.2004.08.016 (2004).
Ikeuchi M. Wang Q, Izumi M, Tani T. Nociceptive sensory innervation of the posterior cruciate ligament in osteoarthritic knees. Archives of Orthopaedic and Trauma Surgery. 2012; 132(6)891-895.
Isaac D, Falode T, Liu P, I′Anson H, Dillow K, Gill P. Accelerated rehabilitation after total knee replacement. Knee. 2005; 12(5):346-350.
Jakob, F., et al., Effects of teriparatide in postmenopausal women with osteoporosis pre-treated with bisphosphonates: 36-month results from the European Forsteo Observational Study. Eur J Endocrinol, 2012, 166(1): p. 87-97.
Jarvinen, J. et al. Association between changes in lumbar Modic changes and low back symptoms over a two-year period. BMC Musculoskelet Disord 16, 98 (2015).
Jarvis, M. F. et al. A-803467, a potent and selective Nav1.8 sodium channel blocker, attenuates neuropathic and inflammatory pain in the rat. Proceedings of the National Academy of Sciences of the United States of America 104, 8520-8525 (2007).
Jensen, O. K. Nielsen, C. V., Sorensen, J. S. & Stengaard-Pedersen, K. Type I Modic changes was a significant risk factor for 1-year outcome in sick-listed low back pain patients: a nested cohort study using magnetic resonance imaging of the lumbar spine. Spine J 14, 2568-2581 (2014).
Jensen, T. S., Karppinen, J., Sorensen, J. S., Niinimaki, J. & Leboeuf-Yde, C. Vertebral endplate signal changes (Modic change): a systematic literature review of prevalence and association with non-specific low back pain. European Spine Journal 17, 1407-1422, doi:10.1007/s00586-008-0770-2 (2008).
Jia, H., et al., Oestrogen and parathyroid hormone alleviate lumbar intervertebral disc degeneration in ovariectomized rats and enhance Wnt/beta-catenin pathway activity. Sci Rep, 2016. 6: p. 27521.
Karlsson J. Sjogren L S, Lohmander L S. Comparison of two hyaluronan drugs and placebo in patients with knee osteoarthritis. A controlled, randomized, double-blind, parallel-design multicentre study. Rheumatology. 2002; 41(11):1240-1248.
Kato, G. et al. Electrophysiological mapping of the nociceptive inputs to the substantia gelatinosa in rat horizontal spinal cord slices. J Physiol 560, 303-315, doi:10.1113/jphysiol.2004.068700 (2004).
Katz, J. N., Lumbar disc disorders and low-back pain: socioeconomic factors and consequences. J Bone Joint Surg Am. 2006. 88 Suppl 2: p. 21-4.
Kc R, Li X, Kroin J S, et al. PKCdelta null mutations in a mouse model of osteoarthritis alter osteoarthritic pain independently of joint pathology by augmenting NGF/TrkA-induced axonal outgrowth. Annals of the rheumatic diseases. 2016; 75(12):2133-2141.
Kim, J.-S. et al. Development of an Experimental Animal Model for Lower Back Pain by Percutaneous Injury-Induced Lumbar Facet Joint Osteoarthritis. J Cell Physiol 230, 2837-2847 (2015).
Kim, J.-S. et al. The rat intervertebral disk degeneration pain model: relationships between biological and structural alterations and pain. Arthritis Res Ther 13, R165 (2011).
Kirkby Shaw, K., Rausch-Derra, L. C. & Rhodes, L. Grapiprant: an EP4 prostaglandin receptor antagonist and novel therapy for pain and inflammation. Veterinary medicine and science 2, 3-9 (2016).
Kobayashi T, Chung U I, Schipani E, et al. PTHrP and Indian hedgehog control differentiation of growth plate chondrocytes at multiple steps. Development (Cambridge, England). 2002:129(12):2977-2986.
Koes, B. W., M. W. van Tulder, and S. Thomas, Diagnosis and treatment of low back pain. BMJ, 2006. 332(7555): p. 1430-4.
Koivisto, K. et al. Efficacy of zoledronic acid for chronic low back pain associated with Modic changes in magnetic resonance imaging. BMC Musculoskelet Disord 15, doi:Artn (410.1186/1471-2474-15-64 (2014).
Koski, A. M., et al., The effectiveness of teriparatide in the clinical practice—attenuation of the bone mineral density outcome by increasing age and bisphosphonate pretreatment. Ann Med, 2013. 45(3): p. 230-5.
Krismer, M., van Tulder, M., Low Back Pain Group of the B. & Joint Health Strategies for Europe, P. Strategies for prevention and management of musculoskeletal conditions. Low back pain (non-specific). Best Pract Res Clin Rheumatol 21, 77-91, doi:10.1016/j.berh.2006.08.004 (2007).
Kroin, J. S., Buvanendran, A., Cochran, E. & Tuman, K. J. Characterization of pain and pharmacologic responses in an animal model of lumbar adhesive arachnoiditis. Spine 30, 1828-1831 (2005).
Kusumbe A P, Ramasany S K, Adams R H. Coupling of angiogenesis and osteogenesis by a specific vessel subtype in bone. Nature. 2014; 507(7492):323-328.
Kwoh C K. OSTEOARTHRITIS Clinical relevance of bone marrow lesions in OA. Nature Reviews Rheumatology. 2013; 9(0):7-8.
Lane N E, Schnitzer T J, Birbara C A, et al. Tanezumab for the Treatment of Pain from Osteoarthritis of the Knee. New England Journal of Medicine. 2010; 363(16):1521-1531.
Langdahl, B. L., et al., Fracture Rate, Quality of Life and Back Pain in Patients with Osteoporosis Treated with Teriparatide: 24-Month Results from the Extended Forsteo Observational Study (ExFOS). Calcif Tissue Int, 2016. 99(3): p. 259-71.
Laslett L L, Dore D A, Quinn S J, et al. Zoledronic acid reduces knee pain and bone marrow lesions over 1 year: a randomised controlled trial. Annals of the Rheumatic Diseases. 2012:71(8):1322-1328.
Laslett L L, Kingsbury S R. Hensor E M A, Bowes M A. Conaghan P G. Effect of bisphosphonate use in patients with symptomatic and radiographic knee osteoarthritis: data from the Osteoarthritis Initiative. Annals of the Rheumatic Diseases. 2014; 73(5):824-830.
Liu, C., Li, Q., Su, Y. & Bao, L. Prostaglandin E2 promotes Na1.8 trafficking via its intracellular RRR motif through the protein kinase A pathway. Traffic (Copenhagen, Denmark) 11, 405-417 (2010).
Lotz, J. C., Fields, A. J. & Liebenberg, E. C. The role of the vertebral end plate in low back pain. Global Spine J 3, 153-164 (2013).
Luoma, K., et al., Low back pain in relation to lumbar disc degeneration. Spine (Phila Pa. 1976), 2000. 25(4): p. 487-92.
Luoma, K., Vehmas, T., Kerttula, L., Gronblad, M. & Rinne, E. Chronic low back pain in relation to Modic changes, bony endplate lesions, and disc degeneration in a prospective MRI study. European spine journal:official publication of the European Spine Society, the European Spinal Deformity Society, and the European Section of the Cervical Spine Research Society 25, 2873-2881 (2016).
Lyu G, Guan Y. Zhang C, et al. TGF-beta signaling alters H4K20me3 status via miR-29 and contributes to cellular senescence and cardiac aging. Nat Commun. 2018; 9(1):2560.
Ma L, Wu J, Jin Q H. The association between parathyroid hormone 1-34 and the Wnt/β-catenin signaling pathway in a rat model of osteoarthritis. Mol Med Rep. 2017:16(6):8799-8807.
Maatta, J. H., Wadge, S., MacGregor, A., Karppinen, J. & Williams, F. M. ISSLS Prize Winner: Vertebral Endplate (Modic) Change is an Independent Risk Factor for Episodes of Severe and Disabling Low Back Pain. Spine (Phila Pa. 1976) 40, 1187-1193, doi:10.1097/BRS.0000000000000937 (2015).
Maher, C., M. Underwood, and R. Buchbinder, Non-specific low back pain. Lancet, 2017. 389(10070): p. 736-747.
Malfait A M. Schnitzer T J. Towards a mechanism-based approach to pain management in osteoarthritis. Nature Reviews Rheumatology. 2013; 9(11):654-664.
Manchikanti, L., Epidemiology of low back pain. Pain Physician, 2000. 3(2): p. 167-92.
Mapp P I, Walsh D A. Mechanisms and targets of angiogenesis and nerve growth in osteoarthritis. Nature Reviews Rheumatology. 2012; 8(7):390-398.
Masuda, K. et al. A novel rabbit model of mild, reproducible disc degeneration by an annulus needle puncture: Correlation between the degree of disc injury and radiological and histological appearances of disc degeneration. Spine 30, 5-14 (2005).
McDougall J J, Andruski B. Schuelert N, Hallgrímsson B, Matyas J R Unravelling the relationship between age, nociception and joint destruction in naturally occurring osteoarthritis of Dunkin Hartley guinea pigs. Pain. 2009; 141(3):222-232.
Mease P J, Hanna S, Frakes E P, Altman R D. Pain Mechanisms in Osteoarthritis: Understanding the Role of Central Pain and Current Approaches to Its Treatment. The Journal of Rheumatology. 2011; 38(8):1546.
Millecamps, M., Czerminski, J. T., Mathieu, A. P. & Stone, L. S. Behavioral signs of axial low back pain and motor impairment correlate with the severity of intervertebral disc degeneration in a mouse model. Spine J 15, 2524-2537, doi:10.1016/j.spinee.2015.08.055 (2015).
Miller R E, Ishihara S, Bhattacharyya B, et al. Chemogenetic Inhibition of Pain Neurons in a Mouse Model of Osteoarthritis. Arthritis & rheumatology (Hoboken, N.J.). 2017; 69(7):1429-1439.
Miller, J. A., C. Schmatz, and A. B. Schultz, Lumbar disc degeneration: correlation with age, sex, and spine level in 600 autopsy specimens. Spine (Phila Pa. 1976). 1988. 13(2): p. 173-8.
Miyakoshi, N., et al., Evidence that anabolic effects of PTH on bone require IGF-I in growing mice. Endocrinology, 2001. 142(10): p. 4349-56.
Miyamoto, S., Yonenobu, K. & Ono, K. Experimental cervical spondylosis in the mouse. Spine 16, S495-500 (1991).
Moore, S. W., Zhang, X., Lynch, C. D. & Sheetz, M. P. Netrin-1 attracts axons through FAK-dependent mechanotransduction. The Journal of neuroscience:the official journal of the Society for Neuroscience 32, 11574-11585 (2012).
Nakao, K. et al. CJ-023,423, a novel, potent and selective prostaglandin EP4 receptor antagonist with antihyperalgesic properties. The Journal of pharmacology and experimental therapeutics 322, 686-694 (2007).
Nakashima, T et al. Evidence for osteocyte regulation of bone homeostasis through RANKL expression. Nat Med 17, 1231-1234 (2011).
Ohtori, S., Inoue, G., Miyagi, M. & Takahashi, K. Pathomechanisms of discogenic low back pain in humans and animal models. Spine J 15, 1347-1355 (2015).
Orth P. Cucchiarini M. Wagenpfeil S. Menger M D. Madry H. PTH [1-34]-induced alterations of the subchondral bone provoke early osteoarthritis. Osteoarthritis and cartilage. 2014; 22(6):813-821.
Orth P, Cucchiarini M, Zurakowski D. Menger M D, Kohn D M. Madry H. Parathyroid hormone [1-34] improves articular cartilage surface architecture and integration and subchondral bone reconstitution in osteochondral defects in vivo. Osteoarthritis and cartilage. 2013; 21(4):614-624.
Papadakis, M., Sapkas, G., Papadopoulos, E. C. & Katonis, P. Pathophysiology and biomechanics of the aging spine. Open Orthop J 5, 335-342. doi:10.2174/1874325001105010335 (2011).
Park, K. W. et al. The axonal attractant Netrin-1 is an angiogenic factor. Proceedings of the National Academy of Sciences of the United States of America 101, 16210-16215 (2004).
Peat G, McCarney R, Croft P. Knee pain and osteoarthritis in older adults: a review of community burden and current use of primary health care. Annals of the Rheumatic Diseases. 2001; 60(2):91-97.
Pfeilschifter J, Laukhuf F, Muller-Beckmann B, Blum W F, Pfister T, Ziegler R. Parathyroid hormone increases the concentration of insulin-like growth factor-I and transforming growth factor beta 1 in rat bone. The Journal of clinical investigation. 1995; 96(2):767-774.
Philipot D, Guerit D. Platano D, et al. p161NK4a and its regulator miR-24 link senescence and chondrocyte terminal differentiation-associated matrix remodeling in osteoarthritis. Arthritis research & therapy. 2014; 16(1):R58.
Pinto, V., Szucs, P., Derkach, V. A. & Safronov, B V. Monosynaptic convergence of C- and Adelta-afferent fibres from different segmental dorsal roots on to single substantia gelatinosa neurones in the rat spinal cord. J Physiol 586, 4165-4177, doi: 10.1113/jphysiol.2008.154898 (2008).
Powell, M. C., et al., Prevalence of lumbar disc degeneration observed by magnetic resonance in symptomless women. Lancet, 1986. 2(8520): p. 1366-7.
Prevalence and most common causes of disability among adults-United States, 2005. MMWR. Morbidity and mortality weekly report. 2009; 58(16):421-426.
Prisby R, Guignandon A, Vanden-Bossche A, et al. Intermittent PTH(1-84) is osteoanabolic but not osteoangiogenic and relocates bone marrow blood vessels closer to bone-forming sites. Journal of bone and mineral research:the official journal of the American Society for Bone and Mineral Research. 2011:26(11):2583-2596.
Pritzker K P, Gay S, Jimenez S A, et al. Osteoarthritis cartilage histopathology: grading and staging. Osteoarthritis and cartilage. 2006; 14(1):13-29.
Qiu T, Wu X, Zhang F, Clemens T L, Wan M, Cao X. TGF-beta type II receptor phosphorylates PTH receptor to integrate bone remodelling signalling. Nature cell biology. 2010; 12(3):224-234.
Rahme, R. & Moussa, R. The Modic vertebral endplate and marrow changes: Pathologic significance and relation to low back pain and segmental instability of the lumbar spine. American Journal of Neuroradiology 29, 838-842, doi:10.3174/ajnr.A0925 (2008).
Rapisarda V. Borghesan M, Miguela V, et al. Integrin Beta 3 Regulates Cellular Senescence by Activating the TGF-beta Pathway. Cell reports. 2017; 18(10):2480-2493.
Reilly K A, Beard D J. Barker K L, Dodd C A F, Price A J, Murray D W. Efficacy of an accelerated recovery protocol for Oxford unicompartmental knee arthroplasty—a randomised controlled trial. Knee. 2005; 12(5):351-357.
Reimann 1, Christensen S B. A histological demonstration of nerves in subchondral bone. Acta orthopaedica Scandinavica. 1977; 48(4):345-352.
Rigaud, M. et al. Species and strain differences in rodent sciatic nerve anatomy: implications for studies of neuropathic pain. Pain 136, 188-201, doi:10.1016/j.pain.2008.01.016 (2008).
Roche B, Vanden-Bossche A, Malaval L, et al. Parathyroid hormone 1-84 targets bone vascular structure and perfusion in mice: impacts of its administration regimen and of ovariectomy. Journal of bone and mineral research:the official journal of the American Society for Bone and Mineral Research. 2014; 29(7):1608-1618.
Rodriguez, A. G., et al., Morphology of the human vertebral endplate. J Orthop Res, 2012. 30(2): p. 280-7.
Rubin, D. I. Epidemiology and risk factors for spine pain. Neurologic Clinics 25, 353-+, doi:10.1016/j.ncl.2007.01.004 (2007).
Saijilafu & Zhou, F.-Q. Genetic study of axon regeneration with cultured adult dorsal root ganglion neurons. J Vis Exp (2012).
Samartzis, D. & Grivas, T. B. Thematic series—Low back pain. Scoliosis and Spinal Disorders 12, doi:ARTN 110.1186/s13013-016-0108-5 (2017).
Sampson E R, Hilton M J, Tian Y, et al. Teriparatide as a chondroregenerative therapy for injury-induced osteoarthritis. Science translational medicine. 2011:3(101):101ra193.
Schaible H G. Mechanisms of chronic pain in osteoarthritis. Current rheumatology reports. 2012; 14(6):549-556.
Schuelert, N. & McDougall, J. J. Involvement of Nav 1.8 sodium ion channels in the transduction of mechanical pain in a rodent model of osteoarthritis. Arthritis Res Ther 14, R5 (2012).
Serafini, T. et al. Netrin-1 is required for commissural axon guidance in the developing vertebrate nervous system. Cell 87. 1001-1014 (1996).
Shu, T., Valentino, K. M., Seaman, C., Cooper, H. M. & Richards, L. J. Expression of the netrin-1 receptor, deleted in colorectal cancer (DCC), is largely confined to projecting neurons in the developing forebrain. The Journal of comparative neurology 416, 201-212 (2000).
Shuang, F. et al. Establishment of a rat model of lumbar facet joint osteoarthritis using intraarticular injection of urinary plasminogen activator. Sci Rep 5, 9828, doi:10.1038/srep09828 (2015).
Soleimani M. Nadri S. A protocol for isolation and culture of mesenchymal stem cells from mouse bone marrow. Nature protocols. 2009; 4(1):102-106.
Southall, M. D. & Vasko, M. R. Prostaglandin receptor subtypes, EP3C and EP4, mediate the prostaglandin E2-induced cAMP production and sensitization of sensory neurons. J Biol Chem 276, 16083-16091 (2001).
Suokas A K. Walsh D A, McWilliams D F, et al. Quantitative sensory testing in painful osteoarthritis: a systematic review and meta-analysis. Osteoarthritis and cartilage. 2012; 2010):1075-1085.
Sun S. Gill S E, Massena de Camin S. Wilson D, McWilliams D F, Walsh D A. Neurovascular invasion at the osteochondral junction and in osteophytes in osteoarthritis. Annals of the rheumatic diseases. 2007; 66(11):1423-1428.
Taher, F. et al. Lumbar degenerative disc disease: current and future concepts of diagnosis and management. Adv Orthop 2012, 970752, doi:10.1155/2012/970752 (2012).
Tang Y, Wu X, Lei W, et al. TGF-beta1-induced migration of bone mesenchymal stem cells couples bone resorption with formation. Nature medicine. 2009; 15(7):757-765.
Telleria-Diaz A, Schmidt M, Kreusch S, et al. Spinal antinociceptive effects of cyclooxygenase inhibition during inflammation: Involvement of prostaglandins and endocannabinoids. Pain. 2010; 148(1):26-35.
Thomas A, Eichenberger G. Kempton C, et al. Recommendations for the treatment of knee osteoarthritis, using various therapy techniques, based on categorizations of a literature review. Journal of geriatric physical therapy (2001). 2009:32(1):33-38.
Tomlinson R E, Li Z, Zhang Q, et al. NGF-TrkA Signaling by Sensory Nerves Coordinates the Vascularization and Ossification of Developing Endochondral Bone. Cell reports. 2016; 16(10):2723-2735.
Traub, R. J. & Mendell, L. M. The spinal projection of individual identified A-delta- and C-fibers. J Neurophysiol 59, 41-55, doi:10.1152/jn.1988.59.1.41 (1988).
Tu, T. et al. CD146 acts as a novel receptor for netrin-1 in promoting angiogenesis and vascular development. Cell research 25, 275-287 (2015).
Usoskin D. Furlan A. Islam S, et al. Unbiased classification of sensory neuron types by large-scale single-cell RNA sequencing. Nature neuroscience. 2015; 18(1):145-153.
Vangsness C T, Jr., Spiker W. Erickson J. A review of evidence-based medicine for glucosamine and chondroitin sulfate use in knee osteoarthritis. Arthroscopy:the journal of arthroscopic & related surgery:official publication of the Arthroscopy Association of North America and the International Arthroscopy Association. 2009; 25(1):86-94.
Videman, T., et al., The long-term effects of physical loading and exercise lifestyles on back-related symptoms, disability, and spinal pathology among men. Spine (Phila Pa. 1976), 1995. 20(6): p. 699-709.
Wan, M., et al., Parathyroid hormone signaling through low-density lipoprotein-related protein 6. Genes Dev, 2008. 22(21): p. 2968-79.
Wang, Y., Videman, T. & Battie, M. C. ISSLS Prize Winner: Lumbar Vertebral Endplate Lesions Associations With Disc Degeneration and Back Pain History. Spine 37, 1490-1496, doi:10.10971BRS.0b013e3182608ac4 (2012).
Wei, F., et al., Pingyangmycin-induced in vivo lumbar disc degeneration model of rhesus monkeys. Spine (Phila Pa. 1976), 2015. 40(4): p. E199-210.
Wein, M. N. and H. M. Kronenberg, Regulation of Bone Remodeling by Parathyroid Hormone. Cold Spring Harb Perspect Med, 2018. 8(8).
Wenger, H. C. and A. S. Cifu, Treatment of Low Back Pain. JAMA, 2017. 318(8): p. 743-744.
Woolf C J, Salter M W. Neuronal Plasticity: Increasing the Gain in Pain. 2000; 288(5472):1765-1768.
Woolf C J. Central sensitization: Implications for the diagnosis and treatment of pain. Pain. 2011:152(3, Supplement):S2-S15.
Wrana J L, Attisano L. Carcamo J, et al. TGF beta signals through a heteromeric protein kinase receptor complex. Cell. 1992; 71(6):1003-1014.
Wright A A, Cook C, Abbott J H. Variables associated with the progression of hip osteoarthritis: a systematic review. Arthritis and rheumatism. 2009; 61(7):925-936.
Xie H, Cui Z, Wang L. et al. PDGF-BB secreted by preosteoclasts induces angiogenesis during coupling with osteogenesis. Nature Medicine. 2014; 20(11):1270-1278.
Xiong, J. et al. Matrix-embedded cells control osteoclast formation. Nat Med 17, 1235-1241 (2011).
Yan J Y, Tian F M, Wang W Y, et al. Parathyroid hormone (1-34) prevents cartilage degradation and preserves subchondral bone micro-architecture in guinea pigs with spontaneous osteoarthritis. Osteoarthritis and cartilage. 2014:22(11):1869-1877.
Yu B, Zhao X, Yang C, et al. Parathyroid hormone induces differentiation of mesenchymal stromal/stem cells by enhancing bone morphogenetic protein signaling. 2012; 27(9):2001-2014.
Yusuf E, Kortekaas M C, Watt I, Huizinga T W J, Kloppenburg M. Do knee abnormalities visualised on MRI explain knee pain in knee osteoarthritis? A systematic review. Annals of the Rheumatic Diseases. 2011:70(1).60-67.
Zhang W, Moskowitz R W, Nuki G, et al. OARSI recommendations for the management of hip and knee osteoarthritis. Part 11: OARSI evidence-based, expert consensus guidelines. Osteoarthritis and cartilage. 2008; 16(2):137-162.
Zhen G, Wen C. Jia X, et al. Inhibition of TGF-beta signaling in mesenchymal stem cells of subchondral bone attenuates osteoarthritis. Nature medicine. 2013:19(6):704-712.
Zhen G H, Cao X. Targeting TGF beta signaling in subchondral bone and articular cartilage homeostasis. Trends in Pharmacological Sciences. 2014:35(5):227-236.
Zheng, L., et al., Ciliary parathyroid hormone signaling activates transforming growth factor-beta to maintain intervertebral disc homeostasis during aging. Bone Res, 2018. 6: p. 21.
Zhong, R., et al., The effects of intervertebral disc degeneration combined with osteoporosis on vascularization and microarchitecture of the endplate in rhesus monkeys. Eur Spine J, 2016. 25(9): p. 2705-15.
Zhou, M., et al., Mortality, morbidity, and risk factors in China and its provinces, 1990-2017: a systematic analysis for the Global Burden of Disease Study 2017. Lancet. 2019.
Zhou, Z., et al, Intervertebral disk degeneration: T1rho M R imaging of human and animal models. Radiology. 2013. 268(2): p. 492-500.
Zhu S, Zhu J, Zhen G. et al. Subchondral bone osteoclasts induce sensory innervation and osteoarthritis pain. The Journal of clinical investigation. 2019:129(3):1076-1093.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A method for treating low back pain (LBP) and/or osteoarthritic pain in a subject in need of treatment thereof, the method comprising administering to the subject a composition comprising a recombinant parathyroid hormone (PTH) and a pharmaceutically acceptable carrier.

2. The method of claim 1, wherein the low back pain comprises a nonspecific low back pain.

3. The method of claim 1 or claim 2, wherein administering the composition comprising the recombinant parathyroid hormone (PTH) inhibits osteoclast activity-induced sensory innervation in a vertebral endplate of the subject.

4. The method of claim 1 or claim 2, wherein administering the composition comprising the recombinant parathyroid hormone (PTH) increases the intervertebral disc (IVD) space by decreasing the volume and porosity of sclerotic endplates.

5. The method of claim 1 or claim 2, wherein administering the composition comprising recombinant parathyroid hormone (PTH) prevents endplate remodeling and sclerosis.

6. The method of claim 1 or claim 2, wherein administering the composition comprising the recombinant parathyroid hormone (PTH) reduces sensory nerve fibers.

7. The method of claim 1 or claim 2, wherein administering the composition comprising the recombinant parathyroid hormone (PTH) reduces the porosity of sclerotic endplates.

8. The method of any one of claims 1-7, wherein administering the composition comprising the recombinant parathyroid hormone (PTH) treats the osteoarthritic pain by one or more of inhibition of nerve innervation, inhibition of subchondral bone deterioration, inhibition of articular cartilage degeneration, attenuation of joint degeneration, decelerating subchondral bone deterioration, and sustaining of subchondral bone microarchitecture by remodeling.

9. The method of any one of claims 1-8, further comprising administering at least one other agent in combination with administering the composition comprising the recombinant parathyroid hormone (PTH).

10. The method of claim 9, wherein the at least one other agent is selected from paracetamol, an opioid, a non-steroidal anti-inflammatory drug, a skeletal muscle relaxant, a triptan, an α2-agonist, a local anesthetic, a tricyclic antidepressant, a benzodiazepine, a steroid, a visco supplement, and combinations thereof.

11. The method of any one of claims 1-10, wherein the low back pain is associated with one or more of spine degeneration, lumbar disc herniation (LDH), scoliosis, cancer, and an infection.

12. The method of any one of claims 1-11, wherein the recombinant PTH comprises a full-length PTH protein or a fragment of PTH.

13. The method of claim 1, wherein the recombinant parathyroid hormone comprises teriparatide (PTH(34′)).

14. The method of claim 1, wherein the recombinant parathyroid hormone comprises an intact parathyroid hormone (iPTH).

15. The method of any one of claims 1-14, wherein the composition is administered to the subject at least once a day.

16. Use of a recombinant parathyroid hormone to treat low back pain (LBP) or osteoarthritic pain in a subject in need thereof.
